(19) United States
(12) Patent Application Publication
(10) Pub. No.: US 2018/0265467 A1
(43) Pub. Date: Sep. 20, 2018

(54) PYRAZOLIDINE DERIVATIVES AND RELATED COMPOUNDS

(71) Applicant: SELVITA S.A., Kralow (PL)

(72) Inventors: Michal GALEZOWSKI, Skiemiewice (PL); Andrzej GONDELA, Tychy (PL); Oleksandr LEVENETS, Krakow (PL); Joanna FOGT, Leszno (PL); Lukasz DUDEK, Stalowa Wola (PL); Alicja OBARA, Kielce (PL); Jakub WOYCIECHOWSKI, Bolechowice (PL); Marta SOWINSKA, Miastkow (PL); Marcin KROL, Kralow (PL); Artur BIELA, Kralow (PL); Tomasz RSYMSKI, Kralow (PL)

(73) Assignee: SELVITA S.A., Kralow (PL)

(21) Appl. No.: 15/762,191

(22) PCT Filed: Sep. 29, 2016
(86) PCT No.: PCT/EP2016/073182

§ 371 Date: Mar. 22, 2018

(30) Foreign Application Priority Data
Sep. 30, 2015 (EP) 15460091.0

(51) Int. Cl. C07D 207/36; C07D 409/06; A61K 31/4155; C07D 409/14; C07D 405/10; C07D 231/44; A61K 31/4152; A61K 31/422; A61K 31/4439; C07D 401/10; C07D 403/10; A61K 31/4015; A61K 31/5377; A61K 31/553; A61K 31/454; C07D 498/10; C07D 491/08; C07D 405/06; C07D 401/06; A61P 35/00

(52) U.S. Cl. CPC C07D 207/36; C07B 2200/09; A61K 31/4155; C07D 409/14; C07D 405/10; C07D 231/44; A61K 31/4152; A61K 31/422; A61K 31/4439; C07D 401/10; C07D 403/10; A61K 31/4015; A61K 31/5377; A61K 31/553; A61K 31/454; C07D 498/10; C07D 491/08; C07D 405/06; C07D 401/06; A61P 35/00; C07B 2200/07; C07D 409/06

(57) ABSTRACT

The present invention relates to pyrazolidine derivatives and related compounds, methods of preparing said compounds, pharmaceutical compositions and combinations comprising said compounds and the use of such compounds and pharmaceutical compositions for the prophylaxis and treatment of medical conditions that can be affected by inhibiting lactate dehydrogenase (LDH), in particular LDHA and/or LDHB.

### FIELD OF THE INVENTION

The present invention relates to pyrazolidine derivatives and related compounds, methods of preparing said compounds, pharmaceutical compositions and combinations comprising said compounds and the use of such compounds and pharmaceutical compositions for the prophylaxis and treatment of medical conditions that can be affected by inhibiting lactate dehydrogenase (LDH), in particular LDHA and/or LDHB.

### BACKGROUND OF THE INVENTION

Glycolysis is a non-oxidative metabolic pathway in which glucose is degraded by cells to generate ATP (adenosine triphosphate), i.e. energy. While normal, i.e. healthy cells are usually favoring this pathway for generating ATP only under anaerobic conditions, many cancer cells generate ATP—even in the presence of oxygen—from glucose via glycolysis; the glycolytic rate can be up to 200 times greater in malignant rapidly-growing tumor cells than in healthy cells. This switch of energy metabolism in cancer cells to the process of “aerobic glycolysis” is known as the “Warburg Effect” (D. G. Brooke et al., Biorganic & Medicinal Chemistry 22 (2014) 1029-1039; T. V. Pyrkov et al., ChemMedChem 2013, 8, 1322-1329).

Increased glycolysis is one of the elements of the metabolic shift tightly linked to the activation of oncogenes and of silencing of tumor suppressors. Altered metabolism in cancer cells is as an attractive target for novel anti-cancer treatments. Inhibition of enzymes in the glycolytic path is expected to result in impaired production of energy that is needed for cancer cells to survive, progress and invade healthy tissues (Doherty, J. R., & Cleveland, J. L., The Journal of Clinical Investigation, 2013, 123(9), 3685-92.; Vander Heiden, M. G., Nature Reviews. Drug Discovery, 2011, 10(9), 671-84).

In the final step of glycolysis pyruvate is converted into lactate which reaction is catalyzed by the enzymes of the lactate dehydrogenase (LDH) family, i.e. lactate dehydrogenase A (LDHA) and B (LDHB), with the reduced form of nicotinamide adenine dinucleotide (NADH) acting as a cofactor. In human beings LDHA and LDHB form a tetrameric enzyme comprising combinations of two isoforms. LDHA predominates in the skeletal muscles and is mostly involved in anaerobic reduction of pyruvate whereas LDHB predominates in the cardiac muscles and catalyzes the aerobic oxidation of pyruvate.

Human cancers exhibit higher LDHA levels compared to normal tissues and elevated LDHA levels are associated with many cancers and are predictors of poor survival (Girgis, H., et al., Molecular Cancer, 2014, 13(1), 101).

Tumor specific deregulation of LDHA occurs in response to tumor environment and oncogenic signals such as c-Myc and KRAS, loss of p53, activation of PI3K pathway and HIF1α (Allison, S. J., et al., Oncogenesis, 2014, 3(5), e102; McCleland, M. L., et al., Clinical Cancer Research, 2013, 19(4), 773-84; Shim, H., et al., Proceedings of the National Academy of Sciences (PNAS), 1997, 94(13), 6658-63; Sonveaux, P., et al., The Journal of Clinical Investigation, 2008, 118(12), 3930-42).

Up-regulation of LDHA ensures efficient glycolytic metabolism when oxygen is limited for tumor cells and reduces oxygen dependency. Various studies support the essential role of both LDHA and glycolysis in tumor development (Cairns, R. A., et al., Nature Reviews. Cancer, 2011, 11(2), 85-95; Doherty, J. R., & Cleveland, J. L., The Journal of Clinical Investigation, 2013, 123(9), 3685-92; Kroemer, G., & Pouyssegur, J., Cancer Cell, 2008, 13(6), 472-82; Schulze, A., & Harris, A. L. (2012), Nature, 2012, 491(7424), 364-73). For example, selective knockdown of LDHA has been shown to result in reduced viability of cancer cells in vitro and in vivo (Arseneault, R., et al., Cancer Letters 2013, doi:10.1016/j.canlet.2013.03.034; Chesnelong, C., et al., Neuro-Oncology, 2014, 16(5), 686-95; Sheng, S. L., et al., The FEBS Journal, 2012, 279(20), 3898-910; Xie, H., et al., Cell Metabolism, 2014, 19, 1-15).

LDHB can also be upregulated in cancer showing preference for glycolytic metabolism such as triple-negative breast cancer (McCleland, K. L., et al., Cancer Research, 2012, 72(22), 5812-23). Moreover, LDHB knockdown in lung adenocarcinoma cells with mutations in KRAS cells and triple-negative breast cancer cells results in reduced proliferation both in vitro and in vivo (McCleland, M. L., et al., Clinical Cancer Research 2013, 19(4), 773-84).

Pharmacological inhibition of LDHA and LDHB is of great interest for cancer treatment. The safety of this approach can be hypothesized based on the fact that hereditary loss of LDHA and LDHB in human individuals results in only mild phenotypes, suggesting that inhibition of the enzyme will not lead to significant intolerable side-effects (Kanno, T., et al., Clinica Chimica Acta; International Journal of Clinical Chemistry, 1988, 173(1), 89-98; Kanno, T., et al., Clinica Chimica Acta; International Journal of Clinical Chemistry, 1980, 108(2), 267-76). Several series of small molecules targeting LDHA and LDHB have been reported (Billiard, J., et al., Cancer & Metabolism, 2013, 1(1), 19; Dragovich, et al., Bioorganic & Medicinal Chemistry Letters, 2014, 24(16), 3764-71; Dragovich, P. S., et al., Bioorganic & Medicinal Chemistry Letters, 2013, 23(11), 3186-94; Fauber, B. P., et al., Bioorganic & Medicinal Chemistry Letters, 2013, 23(20), 5533-9; Granchi, C., et al., Journal of Medicinal Chemistry, 2011, 54(6), 1599-612; Granchi, C., et al., Bioorganic & Medicinal Chemistry Letters, 2011, 21(24), 7331-6; Kohlmann, A., et al., Journal of Medicinal Chemistry, 2013, 56(3), 1023-40; Moorhouse, A. D., et al., Chemical Communications, 2011, 47(1), 230-2; Rodríguez-Páez, L., et al., Journal of Enzyme Inhibition and Medicinal Chemistry, 2011, 26(4), 579-86; Ward, R. A., et al., Journal of Medicinal Chemistry, 2012, 55(7), 3285-306).

Increased lactate levels produced by LDH are also characteristics of inflammatory microenvirnoment. Lactate and lactic acid influence cytokine production and motility of immune cells such as CD8+ and CD4+ T cells. Targeting LDH may provide a novel method for the treatment of chronic inflammatory disorders characterized by persistent T cells infiltrates (Haas et al. 2015).

LDH produced lactate creates tumor microenvironment that promotes immune evasion of cancer cells. LDH deficient tumors were characterized by increased natural killer (NK)-mediated cytotoxicity and reduced numbers of myeloid-derived suppressor cells (Husain et al. 2013; Crane et al. 2014).

These studies provide also a rationale for development of LDH inhbitors as a strategy for counteracting of NK-mediated immunosurveillance.

In summary, LDHA and LDHB are attractive targets for the development of new therapeutic agents for use against hypoxic tumors and tumors that display strong glycolytic phenotypes. The unmet need underlying the present invention is to provide LDHA and LDHB inhibitors for the prophylaxis and treatment of oncogenic but also autoimmune, autoinflammatory, metabolic disorders, such as Addison's disease, celiac disease, dermatomyositis, Graves' disease, Hashimoto's thyroiditis, multiple sclerosis, myasthenia gravis, pernicious anemia, reactive arthritis, rheumatoid arthritis, Sjogren syndrome, systemic lupus erythematosus, type I diabetes and infective diseases such as Plasmodium falciparum malaria and their consecutive complication and disorders. Such inhibitors may not only be used as single actives in the prophylaxis and treatment of such diseases but also in combination with other pharmacological active compounds, for instance in combination with immunomodulatory small molecules and biologics.

### DESCRIPTION OF THE INVENTION

It is an object of the present invention to provide inhibitors of lactate dehydrogenase (LDH), in particular of LDHA and/or LDHB wherein that inhibitors may be useful for the prevention and/or treatment of medical conditions, disorders and/or diseases that are affected by LDH activity, in particular by LDHA and/or LDHB activity. It is a particular object of the present invention to provide such inhibitors for the treatment of hyperproliferative disorders, in particular cancer diseases

The object has surprisingly been solved by compounds of formula (Ia) and/or (Ib) and/or (Ic)

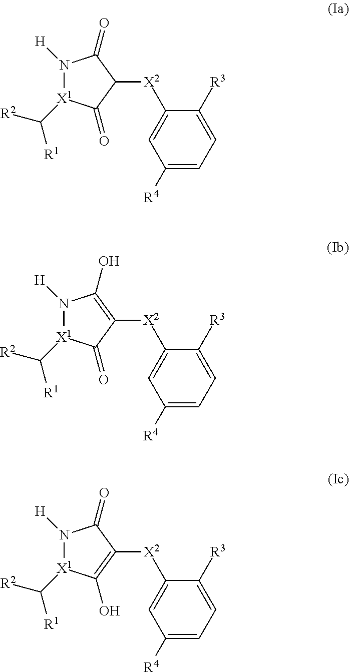

wherein
X¹ denotes N or CH;
X² denotes S or O;
R¹ denotes H, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y};
R² denotes Ar^{X}, Ar^{X}—Ar^{Y}, Ar^{X}-Hetar^{Y}, Ar^{X}-Hetcyc^{Y}, Ar^{X}-L^{Z}-Ar^{Y}, Ar^{X}-LA^{Z}-Ar^{Y}, Ar^{X}-L^{Z}-Hetar^{Y}, Ar^{X}-LA^{Z}-Hetar^{Y}, Ar^{X}-L^{Z}-Hetcyc^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-L^{Z}-Ar^{Y}, Hetar^{X}-LA^{Z}-Ar^{Y}, Hetar^{X}-L^{Z}-Hetar^{Y}, Hetar^{X}-LA^{Z}-Hetar^{Y}, Hetar^{X}-L^{Z}-Hetcyc^{Y}, Hetar^{X}-LA^{Z}-Hetcyc^{Y};
R³ denotes Hal, —CN, —NO₂;
R⁴ denotes H, Hal, LA^{X}, CA^{X}, —CN, NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8};
Ar^{X} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{X1}, R^{X2}, R^{X3};
Ar^{Y} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{Y1}, R^{Y2}, R^{Y3};
Hetar^{X} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or tri-substituted with independently from each other R^{X1}, R^{X2}, R^{X3};
Hetar^{X} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or tri-substituted with independently from each other R^{Y1}, R^{Y2}, R^{Y3};
Hetcyc^{X} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{X4}, R^{X5}, R^{X6};
Hetcyc^{Y} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{Y4}, R^{Y5}, R^{Y6};
R^{X1}, R^{X2}, R^{X3} denote independently from each other H, Hal, LA^{X}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8} OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—SO₂—R^{X9}, —NR^{X7}—SO₂—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8} or
two of R^{X1}, R^{X2}, R^{X3} form a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), —O— wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched C₁₋₆-alkyl or ═O (oxo);
R^{X4}, R^{X5}, R^{X6} denote independently from each other H, Hal, LA^{X}, CA^{X}, —CN, NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O);
R^{Y1}, R^{Y2}, R^{Y3} denote independently from each other H, Hal, LA^{Y}, CA^{Y}, —CN, NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, S(═O)—R^{Y9}, —SO₂—R^{Y9}, —NH₂, —NHR^{Y7}, —NR^{Y7}R^{Y8}, OH, O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8},
   or
two of R^{Y1}, R^{Y2}, R^{Y3} form a divalent alkylene chain with 3, 4, 5 chain carbon atoms wherein 1 or 2 non-adjacent CH₂ groups of the divalent alkylene chain may be replaced independently from each other by —N(H)—, —N(C₁₋₆-alkyl)-, —N(—C(═O)—C₁₋₄-alkyl), —O— wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched C₁₋₆-alkyl or ═O (oxo);
R^{Y4}, R^{Y5}, R^{Y6} denote independently from each other H, Hal, LA^{Y}, CA^{Y}, —CN, NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, S(═O)—R^{Y9}, —SO₂—R^{Y9}, —NH₂, —NHR^{Y7}, —NR^{Y7}R^{Y8}, OH, O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, oxo (═O), or
two of R^{Y4}, R^{Y5}, R^{Y6} form together with one carbon atom to which they are both attached to a saturated or partially unsaturated ring system A which ring system A is mono- or bicyclic and has 3, 4, 5, 6, 7, 8, 9, 10 ring atoms and may contain no hetero ring atom or 1, 2, 3 hetero ring atoms selected independently from each other N, O and/or S while the remaining ring atoms are carbon atoms wherein that ring system A may be unsubstituted or mono-, di- or trisubstituted with independently from each other R^{A1}, R^{A2}, R^{A3};
L^{Z} denotes —NH—, —NR^{Z7}—, —NH-LA^{Z}-, —NR^{Z7}-LA^{Z}-;
LA^{X} denotes straight-chain or branched C₁₋₆-alkyl or C₂₋₆-alkenyl that C₁₋₆-alkyl or C₂₋₆-alkenyl may be unsubstituted or mono-, di- or trisubstituted with independently from each other Hal, —CN, NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —C(═O)—NH—NH₂, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O), wherein 1 or 2 non-adjacent CH₂ groups of the C₁₋₆-alkyl radical or the C₂₋₆-alkenyl radical may independently from each other be replaced by O, S, N(H) or N—R^{X7} and/or 1 or 2 non-adjacent CH groups of the C₁₋₆-alkyl radical or the C₂₋₆-alkenyl radical may independently from each other be replaced by N;
LA^{Y} denotes straight-chain or branched C₁₋₆-alkyl which may be unsubstituted or mono-, di- or trisubstituted with independently from each other Hal, —CN, NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, S(═O)—R^{Y9}, —SO₂—R^{Y9}, —NH₂, —NHR^{Y7}, —NR^{Y7}R^{Y8}, OH, O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, oxo (═O), wherein 1 or 2 non-adjacent CH₂ groups of the C₁₋₆-alkyl radical may independently from each other be replaced by O, S, N(H) or N—R^{Y7} and/or 1 or 2 non-adjacent CH groups of the C₁₋₆-alkyl radical may independently from each other be replaced by N;
LA^{Z} denotes a divalent straight-chain or branched C₁₋₆-alkylene radical which alkylene radical may be unsubstituted or mono-, di- or trisubstituted with independently from each other Hal, —CN, NO₂, —SO₂NH₂, —SO₂NHR^{Z7}, —SO₂NR^{Z7}R^{Z8}, —NH—SO₂—R^{Z9}, —NR^{Z7}—SO₂—R^{Z9}, —S—R^{Z9}, S(═O)—R^{Z9}, —SO₂—R^{Z9}, —NH₂, —NHR^{Z7}, —NR^{Z7}R^{Z8}, OH, O—R^{Z9}, —CHO, —C(═O)—R^{Z9}, —COOH, —C(═O)—O—R^{Z9}, —C(═O)—NH₂, —C(═O)—NHR^{Z7}, —C(═O)—NR^{Z7}R^{Z8}, —NH—C(═O)—R^{Z9}, —NR^{Z7}—C(═O)—R^{Z9}, —NH—(C₁₋₃-alkylene)-C(═O)—NH₂, —NH—(C₁₋₃-alkylene)-C(═O)—NHR^{Z7}, —NH—(C₁₋₃-alkylene)-C(═O)—NR^{Z7}R^{Z8}, oxo (═O), wherein 2 or 2 non-adjacent CH₂ groups of that divalent alkylene radical may be replaced independently from each other by O, S or —N(H) and/or 1 or 2 non-adjacent CH groups of that divalent alkylene radical may be replaced by N;
R^{X7}, R^{X8}, R^{X9}, R^{Y7}, R^{Y8}, R^{Y9}, R^{Z7}, R^{Z8}, R^{Z9} denote independently from each other straight-chain or branched C₁₋₆-alkyl, which may be unsubstituted or mono-, di- or trisubstituted with Hal, or a saturated monocyclic carbocycle with 3, 4, 5, 6, 7 carbon atoms,
   or
each pair R^{X7} and R^{X8}; R^{Y7} and R^{Y8}; R^{Z7} and R^{Z8} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{A1}, R^{A2}, R^{A3} denote independently from each other H, Hal, Ar^{X}, Hetar^{X}, Hetcyc^{X}, LA^{X}, CA^{X}, —CN, NO₂, —SO₂NH₂, —SO₂NHR^{X7}, —SO₂NR^{X7}R^{X8}, —NH—SO₂R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O);
CA^{X}, CA^{Y} denote independently from each other a saturated monocyclic carbocycle with 3, 4, 5, 6, 7 carbon atoms which carbocycle may be unsubstituted or mono- or disubstituted with independently from each other R^{CA1}, R^{CA2};
R^{CA1}, R^{CA2} denote independently from each other H, Hal, LA^{X}, —CN, NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O);
Hal denotes F, Cl, Br, I;
   or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios.

For clarity, it will be understood with regard to the various residues, radicals, moieties, groups and substituents Ar^{X}, Ar^{Y}, Hetar^{X}, Hetar^{Y}, Hetcyc^{X}, Hetcyc^{Y}, LA^{X}, LA^{Y}, LA^{Z}, CA^{X}, CA^{Y}, and the like, that in cases where these residues, radicals, moieties, groups or substituents are part of a more complex residue, radical, moiety or substituent, like in Ar^{X}—Ar^{Y}, Ar^{X}-Hetar^{Y}, Ar^{X}-Hetcyc^{Y}, Ar^{X}-L^{Z}-Ar^{Y}, Ar^{X}-LA^{Z}-Ar^{Y}, Ar^{X}-L^{Z}-Hetar^{Y}, Ar^{X}-LA^{Z}-Hetar^{Y}, Ar^{X}-L^{Z}-Hetcyc^{Y}, Ar^{X}-LA^{Z}-Hetcyc^{Y}, Hetar^{X}-Ar^{Y}, Hetar^{X}-Hetar^{Y}, Hetar^{X}-Hetcyc^{Y}, Hetar^{X}-L^{Z}-Ar^{Y}, Hetar^{X}-LA^{Z}-Ar^{Y}, Hetar^{X}-L^{Z}-Hetar^{Y}, Hetar^{X}-LA^{Z}-Hetar^{Y}, Hetar^{X}-L^{Z}-Hetcyc^{Y}, Hetar^{X}-LA^{Z}-Hetcyc^{Y}, the individual definitions of Ar^{X}, Ar^{Y}, Hetar^{X}, Hetar^{Y}, Hetcyc^{X}, Hetcyc^{Y}, LA^{X}, LA^{Y}, LA^{Z}, CA^{X}, CA^{Y}, and the like, as being unsubstituted or substituted with one or more further specified substituents further comprise their capability or feature of being part of the said more complex residue, radical, moiety, group or substituent. Thus, for instance, “Ar^{X}”, when being part of the more complex “Ar^{X}—Ar^{Y}” moiety, is to be understood as (a) being defined individually, i.e. denoting “a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{X1}, R^{X2}, R^{X3}” and (b) additionally being attached to (or substituted with) the “Ar^{Y}” moiety, which in turn is to be understood as (a) being defined individually, i.e. denoting “a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{Y1}, R^{Y2}, R^{Y3}” and (b) additionally being attached to (or substituted with) the “Ar^{X}” moiety. The same systematics applies, mutatis mutandis, to the other more complex residues, radicals, moieties, groups and substituents.

In general, all residues which occur more than once may be identical or different, i.e. are independent of one another. Above and below, the residues and parameters have the meanings indicated for formulas (Ia), (Ib) and (Ic), unless expressly indicated otherwise. Accordingly, the invention relates, in particular, to the compounds of formulas (Ia), (Ib) and (Ic) in which at least one of the said residues has one of the preferred meanings indicated below.

Any of those preferred or particular embodiments of the present invention as specified below and in the claims do not only refer to the specified compounds of formulas (Ia), (Ib) and (Ic) but to derivatives, N-oxides, prodrugs, solvates, tautomers or stereoisomers thereof as well as the physiologically acceptable salts of each of the foregoing, including mixtures thereof in all ratios, too, unless indicated otherwise.

It is to be noted that the compounds of the present invention may exist in any of the tautomeric forms depicted in formulas (Ia), (Ib) and (Ic). It may well be that a particular compound of the present invention be present predominantly or exclusively in one of the three tautomeric forms, (Ia), (Ib) or (Ic); it may, however, also be that this compound or a different compound of the present invention may be present in two or all three tautomeric forms, either in the same relative amount, e.g. in a relative amount of ½ each in case of two tautomeric forms or in a relative amount of ⅓ each in case of all three tautomeric forms; or in different relative amounts (e.g. 0.2:0.8 or 0.1:0.5:0.4). As will be recognized by the person skilled in the art, it may depend on various factors, like, for instance, aggregation form, temperature or solvent, whether a particular compound of the present invention is present in only one of the tautomeric forms or in two or all three of them and in which relative amounts the tautomeric forms are present, if more than one tautomeric form is present at all.

For reasons of clarity and comprehensibility the compounds of formulas (Ia) and/or (Ib) and/or (Ic) may also collectively referred to as “compounds of formula (I)” throughout this specification and the claims.

In a particular embodiment, PE1, the compounds of the present invention are compounds of formula (I)

wherein
X¹ denotes N or CH;
X² denotes S or O;
R¹ denotes H, Ar^{X}, Hetar^{X};
R² denotes Ar^{X}, Ar^{X}-Hetar^{Y}, Ar^{X}-Hetcyc^{Y}, Ar^{X}-L^{Z}-Hetar^{Y}, Ar^{X}-L^{Z}-Hetcyc^{Y}, Hetar^{X};
R³ denotes Hal, —CN, —NO₂;
R⁴ denotes H, Hal;
Ar^{X} denotes a mono- or bicyclic aromatic ring system with 6 or 10 ring carbon atoms which ring system may be unsubstituted or mono- or disubstituted with independently from each other R^{X1}, R^{X2};
Hetar^{X} denotes a mono- or bicyclic aromatic ring system with 5, 6, 7, 8, 9, 10, 11, 12 ring atoms wherein 1 or 2 of said ring atoms is/are a hetero atom(s) selected from N, O and/or S and the remaining are carbon atoms, wherein that aromatic ring system may be unsubstituted or mono- or disubstituted with independently from each other R^{X1}, R^{X2};
Hetar^{Y} denotes a mono- or bicyclic aromatic ring system with 5, 6, 7, 8, 9, 10 ring atoms wherein 1, 2 or 3 of said ring atoms is/are a hetero atom(s) selected from N, O and/or S and the remaining are carbon atoms, wherein that aromatic ring system may be unsubstituted or mono- or disubstituted with independently from each other R^{Y1}, R^{Y2}; Hetcyc^{Y} denotes a saturated or partially unsaturated mono-, bi- or tricyclic heterocycle with 3, 4, 5, 6, 7, 8, 9, 10, 11, 12 ring atoms wherein 1, 2 or 3 ring atom(s) is/are heteroatom(s) selected from N, O and/or S and the remaining ring atoms are carbon atoms, wherein that heterocycle may be unsubstituted or mono- or disubstituted with R^{Y4}, R^{Y5};
R^{X1}, R^{X2} denote independently from each other H, Hal, LA^{X}, —CN, —NO₂, —NH₂, OH, O—R^{X9}, —COOH, —C(═O)—O—R^{X9}, or
R^{X1} and R^{X2} form a divalent alkylene chain with 3, 4, 5 chain carbon atoms wherein 1 or 2 of non-adjacent CH₂ groups of the divalent alkylene chain may be replaced independently from each other by —O—, which divalent alkylene chain may be unsubstituted or mono- or di-substituted with independently from each other straight-chain or branched C₁₋₆-alkyl or ═O (oxo);
R^{Y1}, R^{Y2} denote independently from each other H, Hal, LA^{Y}, OH, O—R^{Y9}, or
R^{Y1} and R^{Y2} form a divalent alkylene chain with 3, 4, 5 chain carbon atoms wherein 1 or 2 non-adjacent CH₂ groups of the divalent alkylene chain may be replaced independently from each other by —O—, which divalent alkylene chain may be unsubstituted or mono- or di-substituted with independently from each other straight-chain or branched C₁₋₆-alkyl or ═O (oxo);
R^{Y4}, R^{Y5} denote independently from each other H, Hal, LA^{Y}, CA^{Y}, —OH, O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9} or
R^{Y4} and R^{Y5} form together with one carbon atom to which they are both attached to a saturated or partially unsaturated ring system A which ring system A is monocyclic and has 4, 5, 6 ring atoms and may contain no hetero ring atom or 1 or 2 hetero ring atoms selected independently from each other N, O and/or S while the remaining ring atoms are carbon atoms wherein that ring system A may be unsubstituted or monosubstituted with R^{A1};
L^{Z} denotes —NH—, —NR^{Z7-}, —NH-LA^{Z}-, —NR^{Z7}-LA^{Z}-;
LA^{X} denotes straight-chain or branched C₁₋₆-alkyl or C₂—6-alkenyl that C₁₋₆-alkyl or C₂—6-alkenyl may be unsubstituted or monosubstituted with independently from each other —COOH, —C(═O)—O—R^{X9}, —C(═O)—NH₂, —C(═O)—NHR^{X7}, —C(═O)—NR^{X7}R^{X8}, —C(═O)—NH—NH₂, and/or mono-, di- or trisubstituted with Hal;
LA^{Y} denotes straight-chain or branched C₁₋₆-alkyl which may be unsubstituted or monosubstituted with independently from each other —NH₂, —NHR^{Y7}, —NR^{Y7}R^{Y8}, OH, O—R^{Y9}, —NH—C(═O)—R^{Y9}, —NR^{Y7}—C(═O)—R^{Y9}, and/or mono-, di- or trisubstituted with Hal;
LA^{Z} denotes a divalent straight-chain or branched C₁₋₆-alkylene radical;
R^{X7}, R^{X8}, R^{X9}, R^{Y7}, R^{Y8}, R^{Y9}, R^{Z7} denote independently from each other straight-chain or branched C₁₋₆-alkyl, which may be unsubstituted or mono-, di- or trisubstituted with Hal;
R^{A1} denotes H, Hal, LA^{X} or CA^{X};
CA^{X}, CA^{Y} denote independently from each other a saturated monocyclic carbocycle with 3, 4, 5, 6, 7 carbon atoms which carbocycle may be unsubstituted or mono- or disubstituted with independently from each other R^{CA1}, R^{CA2};
R^{CA1}, R^{CA2} denote independently from each other H, Hal, LA^{X}; Hal denotes F, Cl, Br, I;
   or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios.

In another particular embodiment of the present invention, PE2, that may optionally be part of the particular embodiment PE1 as well, substituent R³ of formula (I) denotes Cl, Br or NO₂, and substituent R⁴ of formula (I) denotes H, Cl or Br.

In still another particular embodiment of the present invention, PE3, that may optionally be part of the above described particular embodiments PE1 and/or PE2, substituents R¹ and R² of formula (I) are structurally different, i.e. they are not the same moiety or residue. Since both substituents, R¹ and R², are connected to the same sp³-hybridized carbon atom which already bears two further different substituents, i.e. the core heterocyclic ring and hydrogen, respectively, compounds of PE3 have a stereogenic center and therefore exist in at least stereoisomeric forms, unless, however, R¹ is hydrogen or R¹ and/or R² are structurally identical to the core heterocyclic ring.

Yet another particular embodiment of the present invention, PE4, that may optionally be part of the above described particular embodiments PE1 and/or PE2 and/or PE3, comprises compounds of formula (I) wherein
R¹ denotes H, Ar^{X1} or Hetar^{X1}.

In a preferred embodiment, PE4a, of this particular embodiment PE4
R¹ denotes H, Ar^{X1} or Hetar^{X1}; Ar^{X1} denotes phenyl which is unsubstituted or mono-substituted with R^{X1a}; preferably, it denotes unsubstituted phenyl;
Hetar^{X1} denotes a monocyclic aromatic ring system with 5 or 6 ring atoms wherein 1 or 2 of said ring atoms is/are a hetero atom(s) selected from N, O and/or S and the remaining are carbon atoms, wherein that aromatic ring system may be unsubstituted or monosubstituted with independently from each other R^{X1b}; preferably, it denotes unsubstituted thienyl.

R^{X1a} and R^{X1b} denote independently from each other H, Cl or Br.

A particularly preferred embodiment, PE4b, of PE4 or PE4a comprises compounds of the present invention that are at the same time comprised by particular embodiment PE3, i.e. R² is structurally different from R¹ as defined for PE4 or PE4a. In other words, PE4b is a combination of PE3 with either PE4 or PE4a.

Another particular embodiment of the present invention, PE5, which may optionally be part of one or more of the further particular embodiments of the present invention, i.e. PE1, PE2, PE3, PE4, PE4a, PE4b, comprises compounds of formula (I) in which
R² denotes Ar^{X2}, Ar^{X2}-Hetar^{Y2}, Ar^{X2}-Hetcyc^{Y2}, Ar^{X2}-L^{Z2}-Hetar^{Y2}, Ar^{X2}-L^{Z2}-Hetcyc^{Y2}, Hetar^{X2};
Ar^{X2} denotes phenyl or naphthyl which phenyl or naphthyl may be unsubstituted or mono- or disubstituted with independently from each other R^{X1c}, R^{X2c};
Hetar^{X2} denotes a mono aromatic ring system with 5 or 6 ring atoms wherein 1 or 2 of said ring atoms is/are a hetero atom(s) selected from N, O and/or S and the remaining are carbon atoms, wherein that aromatic ring system may be unsubstituted or monosubstituted with independently from each other R^{X1d};
Hetar^{Y2} denotes a monocyclic aromatic ring system with 5 or 6 ring atoms wherein 1 or 2 of said ring atoms is/are a hetero atom(s) selected from N, O and/or S and the remaining are carbon atoms, wherein that aromatic ring system may be unsubstituted or mono- or disubstituted with independently from each other R^{Y1a}, R^{Y2a};
Hetcyc^{Y2} denotes a saturated or partially unsaturated mono- or bicyclic heterocycle with 4, 5, 6, 7 or 8 ring atoms wherein 1 or 2 atom(s) is/are heteroatom(s) selected from N and/or O and the remaining ring atoms are carbon atoms, wherein that heterocycle may be unsubstituted or mono- or disubstituted with R^{Y4a}, R^{Y5a};
L^{Z2} denotes —NH— or —NH-LA^{Z2}-;
R^{X1c}, R^{X2c} denote independently from each other H, Hal, LA^{X2c}, —OCN, —NO₂, —NH₂, OH, O—R^{X9c}, —COOH, —C(═O)—O—R^{X9c}
   or
R^{X1c} and R^{X2c} form a divalent alkylene chain with 3 or 4 chain carbon atoms wherein 2 of non-adjacent CH₂ groups of the divalent alkylene chain may be replaced by —O—;
   R^{X1d} denotes H or Hal;
R^{Y1a}, R^{Y2a} denote independently from each other H, LA^{Y2a}, OH, O—R^{Y9}a;
R^{Y4a}, R^{Y5a} denote independently from each other H, LA^{Y2b}, CA^{Y2}, —OH, —O—R^{Y9a}, —C(═O)—NH₂,
   or
R^{Y4a} and R^{Y5a} form together with one carbon atom to which they are both attached to a saturated ring system A² which ring system A² is monocyclic and has 4 or 5 ring atoms and may contain no hetero ring atom or 1 hetero ring atom being 0 while the remaining ring atoms are carbon atoms;
LA^{X2c} denotes —C₂-alkenyl that is monosubstituted with —C(═O)—O—R^{X9c}—C(═O)—NH₂, —C(═O)—NH—NH₂;
LA^{Y2a} denotes straight-chain or branched C₁₋₄-alkyl;
LA^{Y2b} denotes straight-chain or branched C₁₋₄-alkyl which may be unsubstituted or monosubstituted with —NR^{Y7a}R^{Y8a}, O—R^{Y9a}, —NH—C(═O)—R^{Y9a}, and/or mono-, di- or trisubstituted with Hal;
LA^{Z2} denotes a divalent straight-chain C₁₋₄-alkylene radical;
CA^{Y2} denotes a saturated monocyclic carbocycle with 3, 4, 5, 6, 7 carbon atoms;
R^{Y7a} and R^{Y7b} denote independently from each other straight-chain or branched C₁₋₄-alkyl;
R^{X9c} denotes methyl or ethyl, which may be unsubstituted or mono-, di- or trisubstituted with Hal;
R^{Y9a} denotes straight-chain or branched C₁₋₄-alkyl; Hal denotes F, Cl, Br.

In a preferred embodiment, PE5a, of this particular embodiment PE5, which may optionally be part of the further particular embodiment of the present invention, i.e. PE1, PE2, PE3, PE4, PE4a, PE4b,
R² denotes phenyl, chlorophenyl, 4-chlorophenyl, 3-chlorophenyl, 2-chlorophenyl, bromophenyl, 3-bromophenyl, 4-bromophenyl, methoxyphenyl, 2-methoxyphenyl, 3-methoxyphenyl, 4-methoxyphenyl, aminophenyl, 4-aminophenyl, trifluormethoxyphenyl, 2-trifluormethoxymethyl, nitrophenyl, 2-nitrophenyl, 3-nitrophenyl, 4-nitrophenyl, cyanophenyl, 4-cyanophenyl, 3-cyanophenyl, hydroxyphenyl, 4-hydroxyphenyl, carboxyphenyl (phenyl-COOH), 3-carboxyphenyl, methoxycarbonylphenyl (phenyl-COOCH₃), 3-methoxycarbonylphenyl, methylphenyl, 3-methylphenyl, 3-ethoxy-3-oxo-prop-1-enylphenyl, 4-[3-ethoxy-3-oxo-prop-1-enyl]phenyl, 3-amino-3-oxo-prop-1-enylphenyl, 4-[3-amino-3-oxo-prop-1-enyl]phenyl, 3-hydrazino-3-oxo-prop-1-enylphenyl, 4-[3-hydrazino-3-oxo-prop-1-enyl]phenyl, 1,3-benzodioxol-4-yl, naphthyl, 1-naphthyl; hydroxypyridyl-phenyl, 3-(6-hydroxy-3-pyridyl)phenyl, 4-(6-hydroxy-3-pyridyl)phenyl, pyridylphenyl, 3-(3-pyridyl)phenyl, 3-(4-pyridyl)phenyl, 4-(3-pyridyl)phenyl, 4-(4-pyridyl)phenyl, methoxypyridyl-phenyl, 4-(6-methoxy-3-pyridyl)phenyl, pyrazolylphenyl, 4-(1H-pyrazol-4-yl)phenyl, 3-(1H-pyrazol-4-yl)phenyl, dimethylpyrazolylphenyl, 4-(3,5-dimethyl-1H-pyrazol-4-yl)phenyl, dimethylisoxazolylphenyl, 4-(3,5-dimethyl isoxazol-4-yl)phenyl, 4-(1H-pyrazol-3-yl)phenyl; tetrahydropyranylphenyl, 4-tetrahydropyran-4-ylphenyl, piperidylphenyl], 4-(1-piperidyl)phenyl, hydroxypiperidylphenyl, 4-(4-hydroxy-1-piperidyl)phenyl, methoxypiperidylphenyl, 4-(4-methoxy-1-piperidyl)phenyl, methoxypyrrolidinylphenyl, 4-(3-methoxypyrrolidin-1-yl)phenyl, morpholinophenyl, 3-morpholinophenyl, 4-morpholinophenyl, cyclopropylmorpholinylphenyl, 3-(2-cyclopropylmorpholin-4-yl)phenyl, 4-(2-cyclopropylmorpholin-4-yl)phenyl, trifluoromethylmorpholinphenyl, 4-(2-trifluoromethylmorpholin-4-yl)phenyl, (dimethylamino)methyl-morpholinylphenyl, 3-[2-[(dimethylamino)methyl]morpholin-4-yl]phenyl, 4-[2-[(dimethylamino)methyl]morpholin-4-yl]phenyl, acetamidomethylmorpholinylphenyl, 3-[2-(acetamidomethyl)morpholin-4-yl]phenyl, 4-[2-(acetamidomethyl)morpholin-4-yl]phenyl, methoxymethylmorpholinylphenyl, 3-[2-(methoxymethyl)morpholin-4-yl]phenyl, 4-[2-(methoxymethyl)morpholin-4-yl]phenyl, carbamoylmorpholinylphenyl, 3-(2-carbamoylmorpholin-4-yl)phenyl, 4-(2-carbamoylmorpholin-4-yl)phenyl, dimethylmorpholinylphenyl, 3-(2,2-dimethylmorpholin-4-yl)phenyl, 4-(2,2-dimethylmorpholin-4-yl)phenyl, 3-(2-oxa-5-azabicyclo[2.2.1]heptan-5-yl)phenyl, 4-(2-oxa-5-azabicyclo[2.2.1]heptan-5-yl)phenyl, 3-(2-oxa-5-azabicyclo[2.2.2]octan-5-yl)phenyl, 4-(2-oxa-5-azabicyclo[2.2.2]octan-5-yl)phenyl, 1,4-oxazepanylphenyl, 3-(1,4-oxazepan-4-yl)phenyl, 4-(1,4-oxazepan-4-yl)phenyl, 4-(6-oxa-9-azaspiro[4.5]decan-9-yl)phenyl, 4-(2-oxa-6-azaspiro[3.3]heptan-6-yl)phenyl, 4-(3,6-dihydro-2H-pyran-4-yl)phenyl; pyrazolylaminophenyl, 4-(1H-pyrazol-4-ylamino)phenyl, (2-hydroxy-4-pyridyl)aminophenyl], 4-[(2-hydroxy-4-pyridyl)amino]phenyl], (6-hydroxy-3-pyridyl)aminophenyl, 4-[(6-hydroxy-3-pyridyl)amino]phenyl; oxetan-3-ylaminophenyl, 4-(oxetan-3-ylamino)phenyl, tetrahydrofuran-3-ylaminophenyl, 4-(tetrahydrofuran-3-ylamino)phenyl, tetrahydropyran-4-ylaminophenyl, 4-(tetrahydropyran-4-ylamino)phenyl, tetrahydropyran-3-ylaminophenyl, 4-(tetrahydropyran-3-ylamino)phenyl, tetrahydropyranyl-methylaminophenyl, 4-(tetrahydropyran-4-ylmethylamino)phenyl; thienyl, 2-thienyl, 3-thienyl, chlorothienyl, 5-chloro-2-thienyl, pyridyl, 2-pyridyl, 3-pyridyl, 4-pyridyl.

Still another particular embodiment of the present invention, PE6, that may optionally be part of one or more of other particular embodiments, PE1, PE2, PE3, PE4, PE4a, PE4b, PE5, PE5a, comprises compounds of formula (I) wherein
X¹ denotes N;
X² denotes S.

It is yet another particular embodiment of the present invention, PE7, that comprises a compound selected from the following group, N-oxides thereof and physiologically acceptable salts either of the compound or any of its N-oxides, the group consisting of:
1-[(4-chlorophenyl)(thiophen-3-yl)methyl]-4-[(2-chlorophenyl)sulfanyl]-pyrazolidine-3,5-dione
1-[(3-chlorophenyl)(thiophen-3-yl)methyl]-4-[(2-chlorophenyl)sulfanyl]-pyrazolidine-3,5-dione
1-[(4-bromophenyl)(thiophen-3-yl)methyl]-4-[(2-chlorophenyl)sulfanyl]-pyrazolidine-3,5-dione
4-[(2-chlorophenyl)sulfanyl]-1-{[4-(oxan-4-yl)phenyl](thiophen-3-yl)methyl}pyrazolidine-3,5-dione
4-[(2-chlorophenyl)sulfanyl]-1-{[4-(oxan-4-yl)phenyl](phenyl)methyl}-pyrazolidine-3,5-dione
4-[(2-chlorophenyl)sulfanyl]-1-[(4-methoxyphenyl)(thiophen-3-yl)methyl]-pyrazolidine-3,5-dione
1-[bis(thiophen-3-yl)methyl]-4-[(2-chlorophenyl)sulfanyl]pyrazolidine-3,5-dione
1-[(4-bromophenyl)(phenyl)methyl]-4-[(2-chlorophenyl)sulfanyl]pyrazolidine-3,5-dione
1-[(3-bromophenyl)(phenyl)methyl]-4-[(2-chlorophenyl)sulfanyl]pyrazolidine-3,5-dione
4-[(2-chlorophenyl)sulfanyl]-1-{[4-(dimethyl-1,2-oxazol-4-yl)phenyl](thiophen-3-yl)methyl}pyrazolidine-3,5-dione
4-[(2-chlorophenyl)sulfanyl]-1-{[4-(3,5-dimethyl-1H-pyrazol-4-yl)phenyl]-(thiophen-3-yl)methyl}pyrazolidine-3,5-dione
4-[(2-chlorophenyl)sulfanyl]-1-{[4-(1H-pyrazol-4-yl)phenyl](thiophen-3-yl)methyl}pyrazolidine-3,5-dione
4-[(2-chlorophenyl)sulfanyl]-1-{[4-(1H-pyrazol-3-yl)phenyl](thiophen-3-yl)methyl}pyrazolidine-3,5-dione
4-[(2-chlorophenyl)sulfanyl]-1-{[3-(1H-pyrazol-4-yl)phenyl](thiophen-3-yl)methyl}pyrazolidine-3,5-dione
4-[(2-chlorophenyl)sulfanyl]-1-{[4-(pyridin-4-yl)phenyl](thiophen-3-yl)methyl}pyrazolidine-3,5-dione
4-[(2-chlorophenyl)sulfanyl]-1-{[4-(pyridin-3-yl)phenyl](thiophen-3-yl)methyl}pyrazolidine-3,5-dione
4-[(2-chlorophenyl)sulfanyl]-1-{[4-(2-methoxypyridin-4-yl)phenyl](thiophen-3-yl)methyl}pyrazolidine-3,5-dione
4-[(2-chlorophenyl)sulfanyl]-1-{[4-(3,6-dihydro-2H-pyran-4-yl)phenyl]-(thiophen-3-yl)methyl}pyrazolidine-3,5-dione ethyl (2E)-3-[4-({4-[(2-chlorophenyl)sulfanyl]-3,5-dioxopyrazolidin-1-yl}(thiophen-3-yl)methyl)phenyl]prop-2-enoate
4-[(2-chlorophenyl)sulfanyl]-1-{phenyl[4-(pyridin-3-yl)phenyl]methyl}-pyrazolidine-3,5-dione
4-[(2-chlorophenyl)sulfanyl]-1-{phenyl[4-(pyridin-4-yl)phenyl]methyl}-pyrazolidine-3,5-dione
4-[(2-chlorophenyl)sulfanyl]-1-{[4-(5-hydroxypyridin-2-yl)phenyl](phenyl)-methyl}pyrazolidine-3,5-dione
4-[(2-chlorophenyl)sulfanyl]-1-{phenyl[4-(1H-pyrazol-3-yl)phenyl]-methyl}pyrazolidine-3,5-dione
4-[(2-chlorophenyl)sulfanyl]-1-{phenyl[4-(1H-pyrazol-4-yl)phenyl]-methyl}pyrazolidine-3,5-dione
4-[(2-chlorophenyl)sulfanyl]-1-{phenyl[3-(pyridin-4-yl)phenyl]methyl}-pyrazolidine-3,5-dione
4-[(2-chlorophenyl)sulfanyl]-1-{phenyl[3-(pyridin-3-yl)phenyl]methyl}-pyrazolidine-3,5-dione
4-[(2-chlorophenyl)sulfanyl]-1-{[3-(6-oxo-1,6-dihydropyridin-3-yl)phenyl]-(phenyl)methyl}pyrazolidine-3,5-dione
4-[(2-chlorophenyl)sulfanyl]-1-{phenyl[3-(1H-pyrazol-4-yl)phenyl]-methyl}pyrazolidine-3,5-dione
4-[(2-chlorophenyl)sulfanyl]-1-{[4-(3,6-dihydro-2H-pyran-4-yl)phenyl]-methyl}pyrazolidine-3,5-dione
(2E)-3-[4-({4-[(2-chlorophenyl)sulfanyl]-3,5-dioxopyrazolidin-1-yl}(thiophen-3-yl)methyl)phenyl]prop-2-enamide
3-[4-({4-[(2-chlorophenyl)sulfanyl]-3,5-dioxopyrazolidin-1-yl}(thiophen-3-yl)methyl)phenyl]propanehydrazide
(5S)-5-benzyl-3-[(2-chlorophenyl)sulfanyl]pyrrolidine-2,4-dione
(5R)-5-benzyl-3-[(2-chlorophenyl)sulfanyl]pyrrolidine-2,4-dione
3-[(2-chlorophenyl)sulfanyl]-5-(diphenylmethyl)pyrrolidine-2,4-dione
5-benzyl-3-(2-chlorophenoxy)pyrrolidine-2,4-dione
5-benzyl-3-[(5-bromo-2-chlorophenyl)sulfanyl]pyrrolidine-2,4-dione
5-benzyl-3-[(2-nitrophenyl)sulfanyl]pyrrolidine-2,4-dione
5-[(4-bromophenyl)(phenyl)methyl]-3-[(2-chlorophenyl)sulfanyl]pyrrolidine-2,4-dione
4-[(2-chlorophenyl)sulfanyl]-1-({4-[(oxan-4-yl)amino]phenyl}(phenyl)-methyl)pyrazolidine-3,5-dione
1-[(4-aminophenyl)(phenyl)methyl]-4-[(2-chlorophenyl)sulfanyl]pyrazolidine-3,5-dione
4-[(2-chlorophenyl)sulfanyl]-1-({4-[(oxetan-3-yl)amino]phenyl}(phenyl)-methyl)pyrazolidine-3,5-dione
4-[(2-chlorophenyl)sulfanyl]-1-({4-[(6-oxo-1,6-dihydropyridin-3-yl)amino]-phenyl}(phenyl)methyl)pyrazolidine-3,5-dione
4-[(2-chlorophenyl)sulfanyl]-1-({4-[(2-oxo-1,2-dihydropyridin-4-yl)amino]-phenyl}(phenyl)methyl)pyrazolidine-3,5-dione
4-[(2-chlorophenyl)sulfanyl]-1-[(4-{[(3R)-oxolan-3-yl]amino}phenyl)-(phenyl)methyl]pyrazolidine-3,5-dione
4-[(2-chlorophenyl)sulfanyl]-1-[phenyl({4-[(1H-pyrazol-4-yl)amino]-phenyl})methyl]pyrazolidine-3,5-dione
4-[(2-chlorophenyl)sulfanyl]-1-[(4-{[(3R)-oxan-3-yl]amino}phenyl)-(phenyl)methyl]pyrazolidine-3,5-dione
4-[(2-chlorophenyl)sulfanyl]-1-[(4-{[(3S)-oxan-3-yl]amino}phenyl)-(phenyl)methyl]pyrazolidine-3,5-dione
4-[(2-chlorophenyl)sulfanyl]-1-[(3-{[(oxan-4-yl)methyl]amino}phenyl)-(phenyl)methyl]pyrazolidine-3,5-dione
4-[(2-chlorophenyl)sulfanyl]-1-[(3-{[(3S)-oxolan-3-yl]amino}phenyl)-(phenyl)methyl]pyrazolidine-3,5-dione
4-[(2-chlorophenyl)sulfanyl]-1-({3-[(oxetan-3-yl)amino]phenyl}(phenyl)-methyl)pyrazolidine-3,5-dione
4-[(2-chlorophenyl)sulfanyl]-1-({3-[(6-oxo-1,6-dihydropyridin-3-yl)amino]-phenyl}(phenyl)methyl)pyrazolidine-3,5-dione
4-[(2-chlorophenyl)sulfanyl]-1-[(3-{[(3S)-oxan-3-yl]amino}phenyl)-(phenyl)methyl]pyrazolidine-3,5-dione
4-[(2-chlorophenyl)sulfanyl]-1-({3-[(2-oxo-1,2-dihydropyridin-4-yl)amino]-phenyl}(phenyl)methyl)pyrazolidine-3,5-dione
4-[(2-chlorophenyl)sulfanyl]-1-({4-[(6-oxo-1,6-dihydropyridin-3-yl)amino]-phenyl}(thiophen-3-yl)methyl)pyrazolidine-3,5-dione
4-[(2-chlorophenyl)sulfanyl]-1-({4-[(1H-pyrazol-4-yl)amino]phenyl}(thiophen-3-yl)methyl)pyrazolidine-3,5-dione
4-[(2-chlorophenyl)sulfanyl]-1-({4-[(oxan-4-yl)amino]phenyl}(thiophen-3-yl)methyl)pyrazolidine-3,5-dione
4-[(2-chlorophenyl)sulfanyl]-1-({4-[(oxetan-3-yl)amino]phenyl}(thiophen-3-yl)methyl)pyrazolidine-3,5-dione
(5R)-3-[(2-chlorophenyl)sulfanyl]-5-[(S)-{4-[(oxan-4-yl)amino]phenyl}(phenyl)-methyl]pyrrolidine-2,4-dione
4-[(2-chlorophenyl)sulfanyl]-1-{[4-(morpholin-4-yl)phenyl](phenyl)-methyl}pyrazolidine-3,5-dione
4-[(2-chlorophenyl)sulfanyl]-1-{[4-(1,4-oxazepan-4-yl)phenyl](phenyl)methyl}-pyrazolidine-3,5-dione
4-[(2-chlorophenyl)sulfanyl]-1-{phenyl[4-(piperidin-1-yl)phenyl]methyl}-pyrazolidine-3,5-dione
4-[(2-chlorophenyl)sulfanyl]-1-{[4-(4-methoxypiperidin-1-yl)phenyl](phenyl)-methyl}pyrazolidine-3,5-dione
4-[(2-chlorophenyl)sulfanyl]-1-({4-[(3R)-3-methoxypyrrolidin-1-yl]phenyl}-(phenyl)methyl)pyrazolidine-3,5-dione
4-[(2-chlorophenyl)sulfanyl]-1-({4-[(3S)-3-methoxypyrrolidin-1-yl]-phenyl}(phenyl)methyl)pyrazolidine-3,5-dione
4-[(2-chlorophenyl)sulfanyl]-1-{[4-(4-hydroxypiperidin-1-yl)phenyl]-(phenyl)methyl}pyrazolidine-3,5-dione
4-[(2-chlorophenyl)sulfanyl]-1-[(4-{[(3S)-oxolan-3-yl]amino}phenyl)-(phenyl)methyl]pyrazolidine-3,5-dione
4-[(2-chlorophenyl)sulfanyl]-1-{[3-(1,4-oxazepan-4-yl)phenyl](phenyl)-methyl}pyrazolidine-3,5-dione
4-[(2-chlorophenyl)sulfanyl]-1-{[3-(4-methoxypiperidin-1-yl)phenyl](phenyl)-methyl}pyrazolidine-3,5-dione
4-[(2-chlorophenyl)sulfanyl]-1-{[3-(morpholin-4-yl)phenyl](phenyl)methyl}-pyrazolidine-3,5-dione
4-[(2-chlorophenyl)sulfanyl]-1-{[4-(morpholin-4-yl)phenyl](thiophen-3-yl)methyl}pyrazolidine-3,5-dione
4-[(2-chlorophenyl)sulfanyl]-1-[(4-{2-oxa-6-azaspiro[3.3]heptan-6-yl}-phenyl)(thiophen-3-yl)methyl]pyrazolidine-3,5-dione
4-[(2-chlorophenyl)sulfanyl]-1-[phenyl({4-[2-(trifluoromethyl)morpholin-4-yl]phenyl})methyl]pyrazolidine-3,5-dione
4-[(2-chlorophenyl)sulfanyl]-1-{[3-(2-cyclopropylmorpholin-4-yl)phenyl]-(phenyl)methyl}pyrazolidine-3,5-dione
4-[(2-chlorophenyl)sulfanyl]-1-[(3-{2-oxa-5-azabicyclo[2.2.1]heptan-5-yl}phenyl)(phenyl)methyl]pyrazolidine-3,5-dione
4-[3-({4-[(2-chlorophenyl)sulfanyl]-3,5-dioxopyrazolidin-1-yl}(phenyl)methyl)-phenyl]morpholine-2-carboxamide
4-[(2-chlorophenyl)sulfanyl]-1-{[3-(2,2-dimethylmorpholin-4-yl)phenyl]-(phenyl)methyl}pyrazolidine-3,5-dione
4-[(2-chlorophenyl)sulfanyl]-1-[(4-{3-oxa-8-azabicyclo[3.2.1]octan-8-yl}phenyl)(phenyl)methyl]pyrazolidine-3,5-dione
4-[4-({4-[(2-chlorophenyl)sulfanyl]-3,5-dioxopyrazolidin-1-yl}(phenyl)methyl)-phenyl]morpholine-2-carboxamide
4-[(2-chlorophenyl)sulfanyl]-1-[(4-{6-oxa-9-azaspiro[4.5]decan-9-yl}phenyl)-(phenyl)methyl]pyrazolidine-3,5-dione
4-[(2-chlorophenyl)sulfanyl]-1-[(4-{₂—[(dimethylamino)methyl]morpholin-4-yl}phenyl)(phenyl)methyl]pyrazolidine-3,5-dione
N-({4-[4-({4-[(2-chlorophenyl)sulfanyl]-3,5-dioxopyrazolidin-1-yl}(phenyl)-methyl)phenyl]morpholin-2-yl}methyl)acetamide
4-[(2-chlorophenyl)sulfanyl]-1-{[4-(2,2-dimethylmorpholin-4-yl)phenyl]-(phenyl)methyl}pyrazolidine-3,5-dione
4-[(2-chlorophenyl)sulfanyl]-1-{[4-(2-cyclopropylmorpholin-4-yl)phenyl]-(phenyl)methyl}pyrazolidine-3,5-dione
4-[(2-chlorophenyl)sulfanyl]-1-({4-[2-(methoxymethyl)morpholin-4-yl]phenyl}-(phenyl)methyl)pyrazolidine-3,5-dione
4-[(2-chlorophenyl)sulfanyl]-1-[(4-{2-oxa-5-azabicyclo[2.2.1]heptan-5-yl}phenyl)(phenyl)methyl]pyrazolidine-3,5-dione
4-[(2-chlorophenyl)sulfanyl]-1-[(3-{3-oxa-8-azabicyclo[3.2.1]octan-8-yl}phenyl)(phenyl)methyl]pyrazolidine-3,5-dione
4-[(2-chlorophenyl)sulfanyl]-1-[(3-{₂—[(dimethylamino)methyl]morpholin-4-yl}phenyl)(phenyl)methyl]pyrazolidine-3,5-dione
4-[(2-chlorophenyl)sulfanyl]-1-({3-[2-(methoxymethyl)morpholin-4-yl]-phenyl}(phenyl)methyl)pyrazolidine-3,5-dione
N-({4-[3-({4-[(2-chlorophenyl)sulfanyl]-3,5-dioxopyrazolidin-1-yl}(phenyl)-methyl)phenyl]morpholin-2-yl}methyl)acetamide
4-[(2-chlorophenyl)sulfanyl]-1-{[4-(morpholin-4-yl)phenyl]methyl}pyrazolidine-3,5-dione
(5R)-3-[(2-chlorophenyl)sulfanyl]-5-[(S)-[4-(morpholin-4-yl)phenyl]-(phenyl)methyl]pyrrolidine-2,4-dione
1-benzyl-4-[(2-chlorophenyl)sulfanyl]pyrazolidine-3,5-dione
3-({4-[(2-chlorophenyl)sulfanyl]-3,5-dioxopyrazolidin-1-yl}methyl)benzonitrile
4-[(2-chlorophenyl)sulfanyl]-1-[(3-methylphenyl)methyl]pyrazolidine-3,5-dione
4-[(2-chlorophenyl)sulfanyl]-1-{[2-(trifluoromethoxy)phenyl]methyl}-pyrazolidine-3,5-dione
1-[(2H-1,3-benzodioxol-5-yl)methyl]-4-[(2-chlorophenyl)sulfanyl]pyrazolidine-3,5-dione
4-[(2-chlorophenyl)sulfanyl]-1-[(naphthalen-1-yl)methyl]pyrazolidine-3,5-dione methyl 3-({4-[(2-chlorophenyl)sulfanyl]-3,5-dioxopyrazolidin-1-yl}methyl)-benzoate
4-({4-[(2-chlorophenyl)sulfanyl]-3,5-dioxopyrazolidin-1-yl}methyl)benzonitrile
4-[(2-chlorophenyl)sulfanyl]-1-[(4-hydroxyphenyl)methyl]pyrazolidine-3,5-dione
3-({4-[(2-chlorophenyl)sulfanyl]-3,5-dioxopyrazolidin-1-yl}methyl)benzoic acid
4-[(2-chlorophenyl)sulfanyl]-1-[(5-chlorothiophen-2-yl)methyl]pyrazolidine-3,5-dione
4-[(2-chlorophenyl)sulfanyl]-1-[(thiophen-3-yl)methyl]pyrazolidine-3,5-dione
4-[(2-chlorophenyl)sulfanyl]-1-[(thiophen-2-yl)methyl]pyrazolidine-3,5-dione
4-[(2-chlorophenyl)sulfanyl]-1-[(4-methoxyphenyl)methyl]pyrazolidine-3,5-dione
4-[(2-chlorophenyl)sulfanyl]-1-[(3-methoxyphenyl)methyl]pyrazolidine-3,5-dione
4-[(2-chlorophenyl)sulfanyl]-1-[(2-methoxyphenyl)methyl]pyrazolidine-3,5-dione
4-[(2-chlorophenyl)sulfanyl]-1-[(pyridin-4-yl)methyl]pyrazolidine-3,5-dione
4-[(2-chlorophenyl)sulfanyl]-1-[(pyridin-3-yl)methyl]pyrazolidine-3,5-dione
4-[(2-chlorophenyl)sulfanyl]-1-[(pyridin-2-yl)methyl]pyrazolidine-3,5-dione
1-[(3-chlorophenyl)methyl]-4-[(2-chlorophenyl)sulfanyl]pyrazolidine-3,5-dione
1-[(2-chlorophenyl)methyl]-4-[(2-chlorophenyl)sulfanyl]pyrazolidine-3,5-dione
1-[(4-chlorophenyl)methyl]-4-[(2-chlorophenyl)sulfanyl]pyrazolidine-3,5-dione
4-[(2-chlorophenyl)sulfanyl]-1-[(3-nitrophenyl)methyl]pyrazolidine-3,5-dione
4-[(2-chlorophenyl)sulfanyl]-1-[(2-nitrophenyl)methyl]pyrazolidine-3,5-dione
4-[(2-chlorophenyl)sulfanyl]-1-[(4-nitrophenyl)methyl]pyrazolidine-3,5-dione
1-benzyl-4-[(2-nitrophenyl)sulfanyl]pyrazolidine-3,5-dione
1-benzyl-4-[(5-bromo-2-nitrophenyl)sulfanyl]pyrazolidine-3,5-dione 2-benzyl-4-[(2-bromophenyl)sulfanyl]-5-hydroxy-2,3-dihydro-1H-pyrazol-3-one

As used herein, the following definitions shall apply unless otherwise indicated or defined specifically elsewhere in the description and/or the claims for specific substituents, radicals, groups or moieties.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, such as one or more C═C double bond(s) and/or C≡C triple bond(s), but which is not aromatic (also referred to herein as “carbocycle”, “cycloaliphatic” or “cycloalkyl”), that has a single point of attachment to the rest of the molecule. Unless otherwise specified, aliphatic groups contain 1-8 or 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₇ hydrocarbon that is completely saturated or that contains one or more units of unsaturation, but which is not aromatic, that has a single point of attachment to the rest of the molecule. The term “alkyl” usually refers to a saturated aliphatic and acyclic moiety, while the term “alkenyl” usually refers to an unsaturated alphatic and acyclic moiety with one or more C═C double bonds and the term “alkynyl” usually refers to an aliphatic and acyclic moiety with one or more C≡C triple bonds. Exemplary aliphatic groups are linear or branched, substituted or unsubstituted C₁₋₈-alkyl, C₁₋₆-alkyl, C₁₋₄-alkyl, C₂₋₈-alkenyl, C₂₋₆-alkenyl,

C₂₋₈-alkynyl, C₂₋₆-alkynyl groups and hybrids thereof such as (cycloalkyl)alkyl, (cycloalkenyl)alkyl or (cycloalkyl)alkenyl.

In particular, the term “C₁₋₃-alkyl” refers to alkyl groups, i.e. saturated acyclic aliphatic groups, having 1, 2 or 3 carbon atoms. Exemplary C₁₋₃-alkyl groups are methyl, ethyl, propyl and isopropyl. The term “C₁₋₄-alkyl” refers to alkyl groups having 1, 2, 3 or 4 carbon atoms. Exemplary C₁₋₄-alkyl groups are methyl, ethyl, propyl, isopropyl, butyl, isobutyl, and tert-butyl. The term “C₁₋₆-alkyl” refers to alkyl groups having 1, 2, 3, 4, 5 or 6 carbon atoms. Exemplary C₁₋₆-alkyl groups are methyl, ethyl, propyl, isopropyl, butyl, isobutyl, tert-butyl, n-pentyl, 2-pentyl, n-hexyl, and 2-hexyl. The term “C₁₋₈-alkyl” refers to alkyl groups having 1, 2, 3, 4, 5, 6, 7, or 8 carbon atoms. Exemplary C₁₋₈-alkyl groups are methyl, ethyl, propyl, isopropyl, butyl, isobutyl, tert-butyl, n-pentyl, 2-pentyl, n-hexyl, 2-hexyl n-heptyl, 2-heptyl, n-octyl, 2-octyl, and 2,2,4-trimethylpentyl. Each of these alkyl groups may be straight-chain or -except for C₁-alkyl and C₂-alkyl-branched and may be unsubstituted or substituted with 1, 2 or 3 substituents that may be the same or different and are, if not specified differently elsewhere in this specification, selected from the group comprising halogen, hydroxy, alkoxy, unsubstituted or mono- or di-substituted amino.

In some instances the C₁₋₃-alkyl, C₁₋₄-alkyl, C₁₋₆-alkyl, C₁₋₈-alkyl groups may also comprise those residues in which 1 or 2 of non-terminal and non-adjacent —CH₂— (methylene) groups are replaced by —O—, —S— and/or 1 or 2 non-terminal and non-adjacent —CH₂— or —CH— groups are replaced by —NH— or —N—. These replacements yield, for instance, alkyl groups like —CH₂—CH₂—O—CH₃, —CH₂—CH₂—CH₂—S—CH₃, CH₂—CH₂—NH—CH₂—CH₃, CH₂—CH₂—O—CH₂—CH₂—O—CH₃, CH₂—CH₂—N(CH₃)—CH₂—CH₃, and the like. Further and/or different replacements of —CH— and —CH₂— groups may be defined for specific alkyl substituents or radicals elsewhere in the description and/or the claims.

The term “C₃₋₇-cycloalkyl” refers to a cycloaliphatic hydrocarbon, as defined above, with 3, 4, 5, 6 or 7 ring carbon atoms. C₃₋₇-cycloalkyl groups may be unsubstituted or substituted with—unless specified differently elsewhere in this specification—1, 2 or 3 substituents that may be the same of different and are—unless specified differently elsewhere in this specification—selected from the group comprising C₁₋₆-alkyl, O—C₁₋₆-alkyl (alkoxy), halogen, hydroxy, unsubstituted or mono- or di-substituted amino. Exemplary C₃₋₇-cycloalkyl groups are cyclopropyl, 2-methyl-cyclopropyl, cyclopropenyl, cyclobutyl, cyclobutenyl, cyclopentyl, cyclopentenyl, cyclohexyl, cyclohexenyl, cycloheptyl, cycloheptenyl.

The term “alkoxy” refers to alkyl substituents and residues that are connected to another structural moiety via an oxygen atom (—O—). Sometimes, it is also referred to as “O-alkyl” and more specifically as “O—C₁₋₄-alkyl”, “O—C₁₋₆-alkyl”, “O—C₁₋₈-alkyl”. Like the similar alkyl groups, it may be straight-chain or -except for —O—C₁-alkyl and —O—C₂-alkyl-branched and may be unsubstituted or substituted with 1, 2 or 3 substituents that may be the same or different and are, if not specified differently elsewhere in this specification, selected from the group comprising halogen, unsubstituted or mono- or di-substituted amino. Exemplary alkoxy groups are methoxy, trifluoromethoxy, ethoxy, 2,2,2-trifluoroethoxy, n-propoxy, iso-propoxy, n-butoxy, sec-butoxy, tert-butoxy, n-pentoxy.

The term “alkylene” refers to a divalent alkyl group. An “alkylene chain” is a polymethylene group, i.e., —(CH₂)ₙ—, wherein n is a positive integer, preferably 1, 2, 3, 4, 5 or 6. In the context of the present invention “C₁₋₃-alkylene” refers to an alkylene moiety with 1, 2 and 3, respectively, —CH₂— groups; the term “alkylene”, however, not only comprises linear alkylene groups, i.e. “alkylene chains”, but branched alkylene groups as well. The term “C₁₋₆-alkylene” refers to an alkylene moiety that is either linear, i.e. an alkylene chain, or branched and has 1, 2, 3, 4, 5 or 6 carbon atoms. A substituted alkylene chain is a polymethylene group in which one or more methylene hydrogen atoms are replaced by (or with) a substituent. Suitable substituents include those described herein for a substituted alkyl group. In some instances 1 or 2 non-adjacent methylene groups of the alkylene chain may be replaced by, for instance, 0, S and/or NH or N—C₁₋₄-alkyl. Exemplary alkylene groups are —CH₂—, —CH₂—CH₂—, —CH₂—CH₂—CH₂—CH₂—, —O—CH₂—O—, —O—CH₂—CH₂—O—, —CH₂—NH—CH₂—CH₂—, —CH₂—N(CH₃)—CH₂—CH₂—.

The term “halogen” means F, Cl, Br, or I.

The term “heteroatom” means one or more of oxygen (O), sulfur (S), or nitrogen (N), including, any oxidized form of nitrogen or sulfur, e.g. N-oxides, sulfoxides and sulfones; the quaternized form of any basic nitrogen or a substitutable nitrogen of a heterocyclic or heteroaromatic ring, for example N (as in 3,4-dihydro-2H-pyrrolyl), NH (as in pyrrolidinyl) or N-SUB with SUB being a suitable substituent (as in N-substituted pyrrolidinyl).

The term “aryl” used alone or as part of a larger moiety as in “aralkyl”, “aralkoxy”, or “aryloxyalkyl”, refers to monocyclic, bicyclic and tricyclic ring systems having a total of five to fourteen ring members, that ring members being carbon atoms, wherein at least one ring in the system is aromatic, i.e., it has (4n+₂)π (pi) electrons (with n being an integer selected from 0, 1, 2, 3), which electrons are delocalized over the system, and wherein each ring in the system contains three to seven ring members. Preferably, all rings in the aryl system or the entire ring system are aromatic. The term “aryl” is used interchangeably with the term “aryl ring”. In certain embodiments of the present invention, “aryl” refers to an “aromatic ring system”. More specifically, those aromatic ring systems may be mono-, bi- or tricyclic with 5, 6, 7, 8, 9, 10, 11, 12, 13, 14 ring carbon atoms. Even more specifically, those aromatic ring systems may be mono- or bicyclic with 6, 7, 8, 9, 10 ring carbon atoms. Exemplary aryl groups are phenyl, biphenyl, naphthyl, anthracyl and the like, which may be unsubstituted or substituted with one or more identical or different substituents. Also included within the scope of the terms “aryl” or “aromatic ring system”, as they are used herein, is a group in which an aromatic ring is fused to one or more non-aromatic rings, such as indanyl, phthalimidyl, naphthimidyl, phenanthridinyl, or tetrahydronaphthyl, and the like. In the latter case the “aryl” group or substituent is attached to its pendant group via the aromatic part of the ring system.

The terms “heteroaryl” and “heteroar-”, used alone or as part of a larger moiety, e.g., “heteroaralkyl”, or “heteroaralkoxy”, refer to groups having 3, 4, 5, 6, 7, 8, 9, 10, 11, 12, 13, 14 ring atoms (which atoms are carbon and hetero atoms), preferably 5, 6, or 9 ring atoms; having 6, 10, or 14 π (pi) electrons shared in a cyclic array; and having, in addition to carbon atoms, 1, 2, 3, 4 or 5 heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, furazanyl, pyridyl, pyridazinyl, pyrimidinyl, pyrazinyl, indolizinyl, purinyl, naphthyridinyl, pteridinyl, and pyrrolopyridinyl, in particular pyrrolo[2,3-b]pyridinyl. The terms “heteroaryl” and “heteroar-”, as used herein, also include groups in which a heteroaromatic ring is fused to one or more aryl, cycloaliphatic, or heterocyclyl rings, where the radical or point of attachment is preferably on the heteroaromatic or, if present, the aryl ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. For example, an indolyl ring may be attached via one of the ring atoms of the six-membered aryl ring or via one of the ring atoms of the five-membered heteroaryl ring. A heteroaryl group is optionally mono-, bi- or tricyclic. The term “heteroaryl” is used interchangeably with the terms “heteroaryl ring”, “heteroaryl group”, or “heteroaromatic”, any of which terms include rings that are unsubstituted or substituted with one or more identical or different substituents. The term “heteroaralkyl” refers to an alkyl group substituted by a heteroaryl, wherein the alkyl and heteroaryl portions independently are optionally substituted.

A heteroaryl ring can be attached to its pendant group at any of its hetero or carbon ring atoms which attachment results in a stable structure or molecule: any of the ring atoms may be unsubstituted or substituted.

The structures of typical examples of “heteroaryl” substituents as used in the present invention are depicted below:

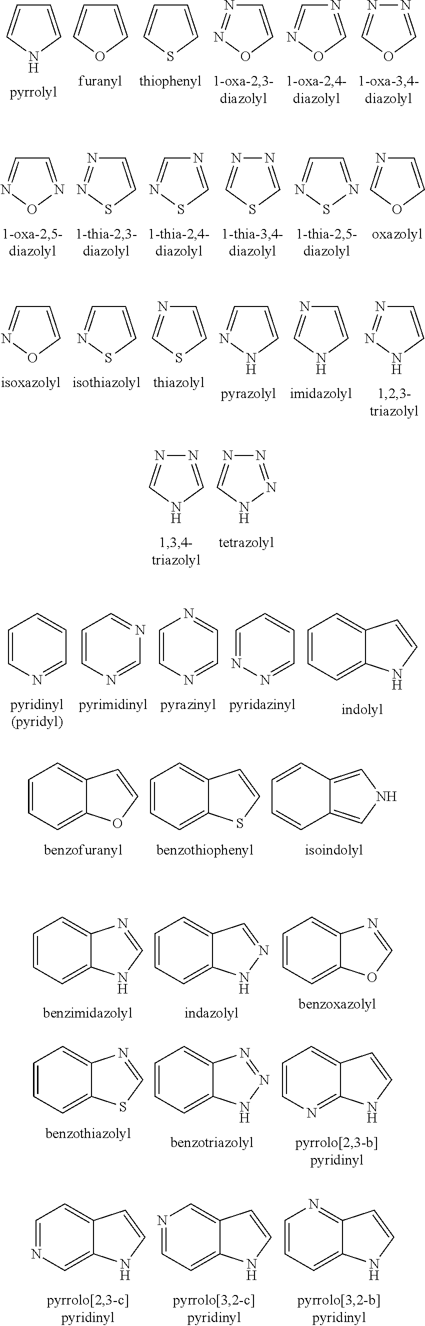

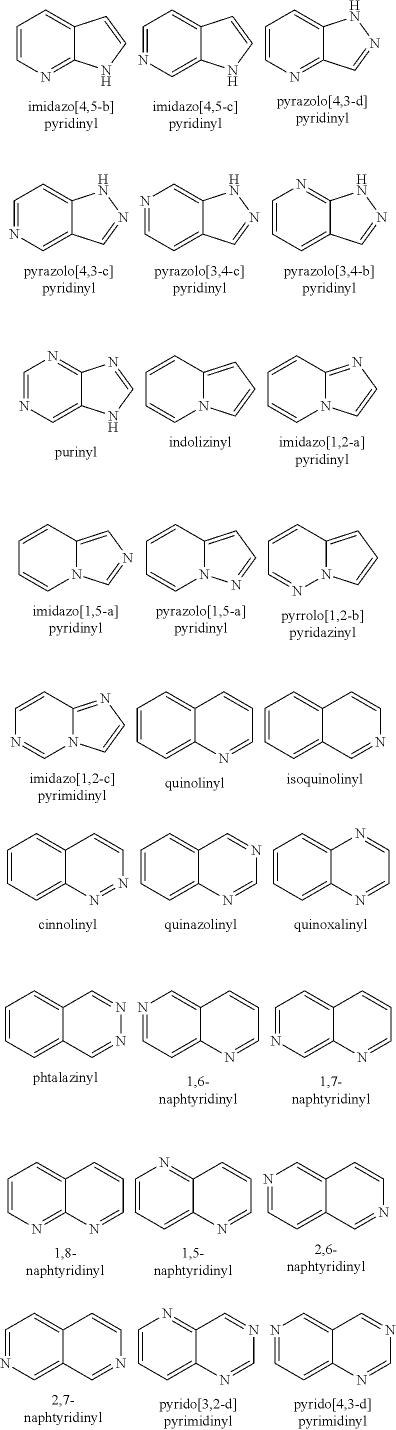

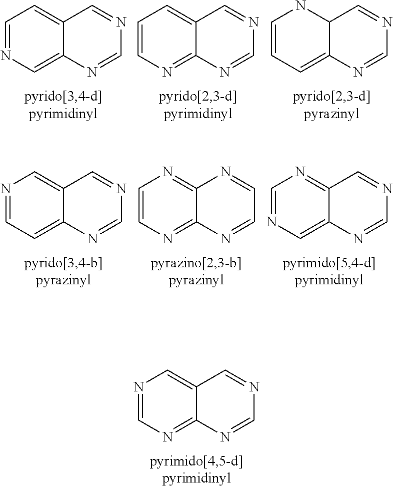

Those heteroaryl substituents can be attached to any pendant group via any of its ring atoms suitable for such an attachment.

As used herein, the terms “heterocycle”, “heterocyclyl”, “heterocyclic radical”, and “heterocyclic ring” are used interchangeably and refer to a stable mono-bi- or tricyclic heterocyclic moiety with 5, 6, 7, 8, 9, 10, 11, 12, 13, 14 ring atoms wherein 1, 2, 3, 4, 5 of said ring atoms are hetero atoms and wherein that heterocyclic moiety is either saturated or partially unsaturated. Preferably, the heterocycle is a stable saturated or partially unsaturated 3-, 4-, 5-, 6-, or 7-membered monocyclic or 7-, 8-, 9-, 10-, or 11-membered bicyclic or 11-, 12-, 13-, or 14-membered tricyclic heterocyclic moiety.

When used in reference to a ring atom of a heterocycle, the term “nitrogen” includes a substituted nitrogen. As an example, in a saturated or partially unsaturated ring having 1-3 heteroatoms selected from oxygen, sulfur or nitrogen, the nitrogen is N (as in 3,4-dihydro-2H-pyrrolyl), NH (as in pyrrolidinyl), or N-SUB with SUB being a suitable substituent (as in N-substituted pyrrolidinyl).

In the context of the term “heterocycle” the term “saturated” refers to a completely saturated heterocyclic system, like pyrrolidinyl, piperidinyl, morpholinyl, and piperidinonyl. With regard to the term “heterocycle” the term “partially unsaturated” refers to heterocyclic systems (i) that contain one or more units of unsaturation, e.g. a C═C or a C=Heteroatom bond, but that are not aromatic, for instance, tetrahydropyridinyl; or (ii) in which a (saturated or unsaturated but non-aromatic) heterocyclic ring is fused with an aromatic or heteroaromatic ring system, wherein, however, the “partially unsaturated heterocycle” is attached to the rest of the molecule (its pendant group) via one of the ring atoms of the “heterocyclic” part of the system and not via the aromatic or heteroaromatic part. This first class (i) of “partially unsaturated” heterocycles may also be referred to as “non-aromatic partially unsaturated” heterocycles. This second class (ii) of “partially unsaturated” heterocycles may also be referred to as (bicyclic or tricyclic) “partially aromatic” heterocycles indicating that at least one of the rings of that heterocycle is a saturated or unsaturated but non-aromatic heterocycle that is fused with at least one aromatic or heteroaromatic ring system. Typical examples of these “partially aromatic” heterocycles are 1,2,3,4-tetrahydroquinolinyl and 1,2,3,4-tetrahydroisoquinolinyl.

A heterocyclic ring can be attached to its pendant group at any heteroatom or carbon atom that results in a stable structure and any of the ring atoms may be unsubstituted or substituted. Examples of such saturated or partially unsaturated heterocyclic radicals include, without limitation, tetrahydrofuranyl, tetrahydropyranyl, tetrahydrothiophenyl pyrrolidinyl, piperidinyl, pyrrolinyl, morpholinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono-, bi- or tricyclic. The term “heterocyclylalkyl” refers to an alkyl group substituted by a heterocyclyl, wherein the alkyl and heterocyclyl portions independently are unsubstituted or substituted.

The term “unsaturated”, as used herein, means that a moiety has one or more units of unsaturation.

As used herein with reference to any rings, ring systems, ring moieties, and the like, the term “partially unsaturated” refers to a ring moiety that includes at least one double or triple bond. The term “partially unsaturated” is intended to encompass rings having multiple sites of unsaturation. In particular, it encompasses (i) non-saturated (mono-, bi- or tricyclic) ring systems without any aromatic or heteroaromatic moiety or part; and (ii) bi- or tricyclic ring systems in which one of the rings of that system is an aromatic or heteroaromatic ring which is fused with another ring that is neither an aromatic nor a heteroaromatic ring, e.g. tetrahydronaphthyl or tetrahydroquinolinyl. The first class (i) of “partially unsaturated” rings, ring systems, ring moieties may also be referred to as “non-aromatic partially unsaturated” rings, ring systems, ring moieties, while the second class (ii) may be referred to as “partially aromatic” rings, ring systems, ring moieties.

As described herein, certain compounds of the invention contain “substituted” or “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure. Unless otherwise indicated, a “substituted” or “optionally substituted” group has a suitable substituent at each substitutable position of the group, and when more than one position in any given structure is substituted with more than one substituent selected from a specified group, the substituent is either the same or different at every position. If a certain group, substituent, moiety or radical is “mono-substituted”, it bears one (1) substituent. If it is “di-substituted”, it bears two (2) substituents, being either the same or different; if it is “tri-substituted”, it bears three (3) substituents, wherein all three are the same or two are the same and the third is different or all three are different from each other. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

In the context of the present invention the term “derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

The compounds of the present invention can be in the form of a prodrug compound. “Prodrugs” and “prodrug compound” mean a derivative that is converted into a biologically active compound according to the present invention under physiological conditions in the living body, e.g., by oxidation, reduction, hydrolysis or the like, each of which is carried out enzymatically, or without enzyme involvement. Examples of prodrugs are compounds, in which the amino group in a compound of the present invention is acylated, alkylated or phosphorylated, e.g., eicosanoylamino, alanylamino, pivaloyloxymethylamino or in which the hydroxyl group is acylated, alkylated, phosphorylated or converted into the borate, e.g. acetyloxy, palmitoyloxy, pivaloyloxy, succinyloxy, fumaryloxy, alanyloxy or in which the carboxyl group is esterified or amidated, or in which a sulfhydryl group forms a disulfide bridge with a carrier molecule, e.g. a peptide, that delivers the drug selectively to a target and/or to the cytosol of a cell. These compounds can be produced from compounds of the present invention according to well-known methods. Other examples of prodrugs are compounds, wherein the carboxylate in a compound of the present invention is for example converted into an alkyl-, aryl-, choline-, amino-, acyloxymethylester, linolenoyl-ester.

The term “solvates” means addition forms of the compounds of the present invention with solvents, preferably pharmaceutically acceptable solvents, that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, e.g. a mono- or dihydrate. If the solvent is alcohol, the solvate formed is an alcoholate, e.g., a methanolate or ethanolate. If the solvent is an ether, the solvate formed is an etherate, e.g., diethyl etherate.

The term “N-oxides” means such compounds of the present invention that contain an amine oxide moiety, i.e. the oxide of a tertiary amine group.

The compounds of formula (I), i.e. compounds of formulas (Ia) and/or (Ib) and/or (Ic) may have one or more centres of chirality. They may accordingly occur in various enantiomeric and diastereomeric forms, as the case may be, and be in racemic or optically active form. The invention, therefore, also relates to the optically active forms, enantiomers, racemates, diastereomers, mixtures thereof in all ratios, collectively: “stereoisomers” for the purpose of the present invention, of these compounds. Since the pharmaceutical activity of the racemates or stereoisomers of the compounds according to the invention may differ, it may be desirable to use a specific stereoisomer, e.g. one specific enantiomer or diastereomer. In these cases, a compound according to the present invention obtained as a racemate—or even intermediates thereof—may be separated into the stereoisomeric (enantiomeric, diastereoisomeric) compounds by chemical or physical measures known to the person skilled in the art. Another approach that may be applied to obtain one or more specific stereoisomers of a compound of the present invention in an enriched or pure form makes use of stereoselective synthetic procedures, e.g. applying starting material in a stereoisomerically enriched or pure form (for instance using the pure or enriched (R)- or (S)-enantiomer of a particular starting material bearing a chiral center) or utilizing chiral reagents or catalysts, in particular enzymes. In the context of the present invention the term “pure enantiomer” usually refers to a relative purity of one enantiomer over the other (its antipode) of equal to or greater than 95%, preferably ≥98%, more preferably ≥98.5%, still more preferably ≥99%.

Thus, for example, the compounds of the invention which have one or more centers of chirality and which occur as racemates or as mixtures of enatiomers or diastereoisomers can be fractionated or resolved by methods known per se into their optically pure or enriched isomers, i.e. enantiomers or diastereomers. The separation of the compounds of the invention can take place by chromatographic methods, e.g. column separation on chiral or nonchiral phases, or by recrystallization from an optionally optically active solvent or by use of an optically active acid or base or by derivatization with an optically active reagent such as, for example, an optically active alcohol, and subsequent elimination of the radical.

In the context of the present invention the term “tautomer” refers to compounds of the present invention that may exist in tautomeric forms and show tautomerism; for instance, carbonyl compounds may be present in their keto and/or their enol form and show keto-enol tautomerism. Those tautomers may occur in their individual forms, e.g., the keto or the enol form, or as mixtures thereof and are claimed separately and together as mixtures in any ratio. The same applies for cis/trans isomers, E/Z isomers, conformers and the like. As pointed out hereinabove, the compounds of the present invention may exist in any of the tautomeric forms depicted in formulas (Ia), (Ib) and (Ic). It may well be that a particular compound of the present invention be present predominantly or exclusively in one of the three tautomeric forms, (Ia), (Ib) or (Ic); it may, however, also be that this compound or a different compound of the present invention may be present in two or all three tautomeric forms, either in the same relative amount, e.g. in a relative amount of ½ each in case of two tautomeric forms or in a relative amount of ⅓ each in case of all three tautomeric forms; or in different relative amounts (e.g. 0.2:0.8 or 0.1:0.5:0.4). As will be recognized by the person skilled in the art, it may depend on various factors, like, for instance, aggregation form, temperature or solvent, whether a particular compound of the present invention is present in only one of the tautomeric forms or in two or all three of them and in which relative amounts the tautomeric forms are present, if more than one tautomeric form is present at all.

The compounds of the present invention can be in the form of a pharmaceutically acceptable salt, a pharmaceutically acceptable solvate, or a pharmaceutically acceptable solvate of a pharmaceutically acceptable salt.

The term “pharmaceutically acceptable salts” refers to salts prepared from pharmaceutically acceptable bases or acids, including inorganic bases or acids and organic bases or acids. In cases where the compounds of the present invention contain one or more acidic or basic groups, the invention also comprises their corresponding pharmaceutically acceptable salts. Thus, the compounds of the present invention which contain acidic groups can be present in salt form, and can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the present invention which contain one or more basic groups, e.g. groups which can be protonated, can be present in salt form, and can be used according to the invention in the form of their addition salts with inorganic or organic acids. Examples of suitable acids include hydrogen chloride, hydrogen bromide, hydrogen iodide, phosphoric acid, sulfuric acid, nitric acid, methanesulfonic acid, p-toluenesulfonic acid, naphthalenedisulfonic acid, sulfoacetic acid, trifluoroacetic acid, oxalic acid, acetic acid, tartaric acid, lactic acid, salicylic acid, benzoic acid, carbonic acid, formic acid, propionic acid, pivalic acid, diethylacetic acid, malonic acid, succinic acid, pimelic acid, fumaric acid, malonic acid, maleic acid, malic acid, embonic acid, mandelic acid, sulfaminic acid, phenylpropionic acid, gluconic acid, ascorbic acid, isonicotinic acid, citric acid, adipic acid, taurocholic acid, glutaric acid, stearic acid, glutamic acid or aspartic acid, and other acids known to the person skilled in the art. The salts which are formed are, inter alia, hydrochlorides, chlorides, hydrobromides, bromides, iodides, sulfates, phosphates, methanesulfonates (mesylates), tosylates, carbonates, bicarbonates, formates, acetates, sulfoacetates, triflates, oxalates, malonates, maleates, succinates, tartrates, malates, embonates, mandelates, fumarates, lactates, citrates, glutarates, stearates, aspartates and glutamates. The stoichiometry of the salts formed from the compounds of the invention may moreover be an integral or non-integral multiple of one.

If the compounds of the present invention simultaneously contain acidic and basic groups in the molecule, the invention also includes, in addition to the salt forms mentioned, inner salts or betaines (zwitterions). The respective salts can be obtained by customary methods which are known to a person skilled in the art, for example by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Therefore, the following items are also in accordance with the invention:

(a) all stereoisomers or tautomers of the compounds, including mixtures thereof in all ratios;
(b) prodrugs of the compounds, or stereoisomers or tautomers of these prodrugs;
(c) pharmaceutically acceptable salts of the compounds and of the items mentioned under (a) and (b);
(d) pharmaceutically acceptable solvates of the compounds and of the items mentioned under (a), (b) and (c);
(e) N-oxides of the compounds and of the items mentioned under (a), (b), (c), and (d).

It should be understood that all references to compounds above and below are meant to include these items, in particular pharmaceutically acceptable solvates of the compounds, or pharmaceutically acceptable solvates of their pharmaceutically acceptable salts.

Furthermore, the present invention relates to pharmaceutical compositions comprising at least one compound of formula (I), or its derivatives, prodrugs, solvates, tautomers or stereoisomers thereof as well as the physiologically acceptable salts of each of the foregoing, including mixtures thereof in all ratios, as active ingredient, together with a pharmaceutically acceptable carrier.

For the purpose of the present invention the term “pharmaceutical composition” refers to a composition or product comprising one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one compound of the present invention and a pharmaceutically acceptable carrier. It may further comprise physiologically acceptable excipients, auxiliaries, adjuvants, diluents and/or additional pharmaceutically active substance other than the compounds of the invention.

The pharmaceutical compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

A pharmaceutical composition of the present invention may additionally comprise one or more other compounds as active ingredients (drugs), such as one or more additional compounds of the present invention. In a particular embodiment the pharmaceutical composition further comprises a second active ingredient or its derivatives, prodrugs, solvates, tautomers or stereoisomers thereof as well as the physiologically acceptable salts of each of the foregoing, including mixtures thereof in all ratios, wherein that second active ingredient is other than a compound of the present invention, i.e. other than a compound of formula (I) as originally disclosed hereinabove; in other words: This second active ingredient does not comprise any of the compounds of formulas (Ia), (Ib) and/or (Ic) as described herein regardless whether or not they are subject matter of the accompanying claims. Preferably, that second active ingredient is a compound that is useful in the treatment, prevention, suppression and/or amelioration of medicinal conditions or pathologies for which the compounds of the present invention are useful as well and which are listed elsewhere hereinbefore or hereinafter. Such combination of two or more active ingredients or drugs may be safer or more effective than either drug or active ingredient alone, or the combination is safer or more effective than it would be expected based on the additive properties of the individual drugs. Such other drug(s) may be administered, by a route and in an amount commonly used contemporaneously or sequentially with a compound of the invention. When a compound of the invention is used contemporaneously with one or more other drugs or active ingredients, a combination product containing such other drug(s) and the compound of the invention—also referred to as “fixed dose combination”—is preferred. However, combination therapy also includes therapies in which the compound of the present invention and one or more other drugs are administered on different overlapping schedules. It is contemplated that when used in combination with other active ingredients, the compound of the present invention or the other active ingredient or both may be used effectively in lower doses than when each is used alone. Accordingly, the pharmaceutical compositions of the present invention include those that contain one or more other active ingredients, in addition to a compound of the invention.

The compounds of the present invention can be used as medicaments. They exhibit pharmacological activity by inhibiting lactate dehydrogenase (LDH), in particular its isoforms LDHA and/or LDHB Thus, they are useful for the treatment, prevention, suppression and/or amelioration of medicinal conditions or pathologies that are affected by LDH activity, in particular by LDHA and/or LDHB activity. The compounds of the present invention are thus particularly useful for the treatment of a hyperproliferative, autoimmune, autoinflammatory, metabolic and infective diseases or disorder, especially of a hyperproliferative disease or disorder. More specifically, they are useful for the treatment of a disorder or disease selected from the group consisting of cancer, in particular central nervous system cancer, cervical cancer, glioblastoma, glioma, myeloid neoplasia, chondrosarcoma, angioimmunoblastic T-cell lymphoma (AITL), cholangiocarcinoma, prostate cancer, leukemia, lymphoma, lymphoid cancer, kidney cancer, hypoxic carcinomas, breast cancer, ovarian cancer, mesothelioma, pancreatic cancer, colon cancer, colorectal cancer, lung cancer, lung adenocarcinomas, non-small cell lung cancer (NSCLC), liver cancer, hepatocellular carcinoma. The compounds of the present invention are aso particularly useful for the prophylaxis and/or treatment of Addison's disease, celiac disease, dermatomyositis, Graves' disease, Hashimoto's thyroiditis, multiple sclerosis, myasthenia gravis, pernicious anemia, reactive arthritis, rheumatoid arthritis, Sjogren syndrome, systemic lupus erythematosus, type I diabetes, malaria, especially Plasmodium falciparum malaria.

The disclosed compounds of formula (I) can be administered and/or used in combination with other known therapeutic agents, including anticancer agents and immunmodulatory agents being it a small chemical or a larger biologic molecule. As used herein, the term “anticancer agent” relates to any agent which is administered to a patient with cancer for the purposes of treating the cancer.

The anti-cancer treatment defined above may be applied as a monotherapy or may involve, in addition to the herein disclosed compounds of formula (I), conventional surgery or radiotherapy or medicinal therapy. Such medicinal therapy, e.g. a chemotherapy or a targeted therapy, may include one or more, but preferably one, of the following anti-tumor agents:

### Alkylating Agents

such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone;
apaziquone, fotemustine, glufosfamide, palifosfamide, pipobroman, trofosfamide, uramustine, TH-302⁴, VAL-083⁴;

### Platinum Compounds

such as carboplatin, cisplatin, eptaplatin, miriplatine hydrate, oxaliplatin, lobaplatin, nedaplatin, picoplatin, satraplatin;

### DNA Altering Agents

such as amrubicin, bisantrene, decitabine, mitoxantrone, procarbazine, trabectedin, clofarabine;
amsacrine, brostallicin, pixantrone, laromustine^{1,3};

### Topoisomerase Inhibitors

such as etoposide, irinotecan, razoxane, sobuzoxane, teniposide, topotecan; amonafide, belotecan, elliptinium acetate, voreloxin;

### Microtubule Modifiers

such as cabazitaxel, docetaxel, eribulin, ixabepilone, paclitaxel, vinblastine, vincristine, vinorelbine, vindesine, vinflunine;
fosbretabulin, tesetaxel;

### Antimetabolites

such as asparaginase³, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur;
doxifluridine, elacytarabine, raltitrexed, sapacitabine, tegafur^{2,3}, trimetrexate;

### Anticancer Antibiotics

such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin;
aclarubicin, peplomycin, pirarubicin;

### Hormones/Antaconists

such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol;
acolbifene, danazol, deslorelin, epitiostanol, orteronel, enzalutamide^{1,3};

### Aromatase Inhibitors

such as aminoglutethimide, anastrozole, exemestane, fadrozole, letrozole, testolactone;
formestane;

### Small Molecule Kinase Inhibitors

such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib;
afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib⁴, cabozantinib S-malate^{1,3}, ibrutinib^{1,3}, icotinib⁴, buparlisib², cipatinib⁴, cobimetinib^{1,3}, idelalisib^{1,3}, fedratinib¹, XL-647⁴;

### Photosensitizers

such as methoxsalen³;
porfimer sodium, talaporfin, temoporfin;

### Antibodies

such as alemtuzumab, besilesomab, brentuximab vedotin, cetuximab, denosumab, ipilimumab, ofatumumab, panitumumab, rituximab, tositumomab,
trastuzumab, bevacizumab, pertuzumab^{2,3};
catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab^{1,2,3}, onartuzumab^{1,3}, racotumomab¹, tabalumab^{1,3}, EMD-525797⁴, nivolumab^{1,3}; Cytokines such as aldesleukin, interferon alfa², interferon alfa2a³, interferon alfa2b^{2,3}; celmoleukin, tasonermin, teceleukin, oprelvekin^{1,3}, recombinant interferon beta-1a⁴;

### Drug Conjugates

such as denileukin diftitox, ibritumomab tiuxetan, iobenguane I1123, prednimustine, trastuzumab emtansine, estramustine, gemtuzumab, ozogamicin, aflibercept;
cintredekin besudotox, edotreotide, inotuzumab ozogamicin, naptumomab estafenatox, oportuzumab monatox, technetium (99mTc) arcitumomab^{1,3} vintafolide^{1,3};

### Vaccines

such as sipuleucel³; vitespen³, emepepimut-S³, oncoVAX⁴, rindopepimut³, troVax⁴, MGN-1601⁴, MGN-1703⁴;

### Miscellaneous

alitretinoin, bexarotene, bortezomib, everolimus, ibandronic acid, imiquimod, lenalidomide, lentinan, metirosine, mifamurtide, pamidronic acid, pegaspargase, pentostatin, sipuleucel³, sizofiran, tamibarotene, temsirolimus, thalidomide, tretinoin, vismodegib, zoledronic acid, vorinostat; celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine⁴,
picibanil⁴, reolysin⁴, retaspimycin hydrochloride^{1,3}, trebananib²³, virulizin⁴, carfilzomib^{1,3}, endostatin⁴, immucothel⁴, belinostat³, MGN-1703⁴; ¹ Prop. INN (Proposed International Nonproprietary Name)² Rec. INN (Recommended International Nonproprietary Names)³ USAN (United States Adopted Name)⁴ no INN.

A further embodiment of the present invention is a process for the manufacture of the pharmaceutical compositions of the present invention, characterized in that one or more compounds according to the invention and one or more compounds selected from the group consisting of solid, liquid or semiliquid excipients, auxiliaries, adjuvants, diluents, carriers and pharmaceutically active agents other than the compounds according to the invention, are converted in a suitable dosage form.

In another aspect of the invention, a set or kit is provided comprising a therapeutically effective amount of at least one compound of the invention and/or at least one pharmaceutical composition as described herein and a therapeutically effective amount of at least one further pharmacologically active substance other than the compounds of the invention. It is preferred that this set or kit comprises separate packs of

a) an effective amount of a compound of formula (I), or its derivatives, prodrugs, solvates, tautomers or stereoisomers thereof as well as the physiologically acceptable salts of each of the foregoing, including mixtures thereof in all ratios, and
b) an effective amount of a further active ingredient that further active ingredient not being a compound of formula (I).

The pharmaceutical compositions of the present invention may be administered by any means that achieve their intended purpose. For example, administration may be via oral, parenteral, topical, enteral, intravenous, intramuscular, inhalant, nasal, intraarticular, intraspinal, transtracheal, transocular, subcutaneous, intraperitoneal, transdermal, or buccal routes. Alternatively, or concurrently, administration may be via the oral route. The dosage administered will be dependent upon the age, health, and weight of the recipient, kind of concurrent treatment, if any, frequency of treatment, and the nature of the effect desired. Parenteral administration is preferred. Oral administration is especially preferred.

Suitable dosage forms include, but are not limited to capsules, tablets, pellets, dragees, semi-solids, powders, granules, suppositories, ointments, creams, lotions, inhalants, injections, cataplasms, gels, tapes, eye drops, solution, syrups, aerosols, suspension, emulsion, which can be produced according to methods known in the art, for example as described below:

Tablets: mixing of active ingredient/s and auxiliaries, compression of said mixture into tablets (direct compression), optionally granulation of part of mixture before compression.

Capsules: mixing of active ingredient/s and auxiliaries to obtain a flowable powder, optionally granulating powder, filling powders/granulate into opened capsules, capping of capsules.

Semi-solids (ointments, gels, creams): dissolving/dispersing active ingredient/s in an aqueous or fatty carrier; subsequent mixing of aqueous/fatty phase with complementary fatty/aqueous phase, homogenization (creams only).

Suppositories (rectal and vaginal): dissolving/dispersing active ingredient/s in carrier material liquified by heat (rectal: carrier material normally a wax; vaginal: carrier normally a heated solution of a gelling agent), casting said mixture into suppository forms, annealing and withdrawal suppositories from the forms.

Aerosols: dispersing/dissolving active agent/s in a propellant, bottling said mixture into an atomizer.

In general, non-chemical routes for the production of pharmaceutical compositions and/or pharmaceutical preparations comprise processing steps on suitable mechanical means known in the art that transfer one or more compounds of the invention into a dosage form suitable for administration to a patient in need of such a treatment. Usually, the transfer of one or more compounds of the invention into such a dosage form comprises the addition of one or more compounds, selected from the group consisting of carriers, excipients, auxiliaries and pharmaceutical active ingredients other than the compounds of the invention. Suitable processing steps include, but are not limited to combining, milling, mixing, granulating, dissolving, dispersing, homogenizing, casting and/or compressing the respective active and nonactive ingredients. Mechanical means for performing said processing steps are known in the art, for example from Ullmann's Encyclopedia of Industrial Chemistry, 5th Edition. In this respect, active ingredients are preferably at least one compound of the invention and optionally one or more additional compounds other than the compounds of the invention, which show valuable pharmaceutical properties, preferably those pharmaceutical active agents other than the compounds of the invention, which are disclosed herein.

Particularly suitable for oral use are tablets, pills, coated tablets, capsules, powders, granules, syrups, juices or drops, suitable for rectal use are suppositories, suitable for parenteral use are solutions, preferably oil-based or aqueous solutions, furthermore suspensions, emulsions or implants, and suitable for topical use are ointments, creams or powders. The compounds of the invention may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, dyes, flavours and/or a plurality of further active ingredients, for example one or more vitamins.

Suitable excipients are organic or inorganic substances, which are suitable for enteral (for example oral), parenteral or topical administration and do not react with the compounds of the invention, for example water, vegetable oils, benzyl alcohols, alkylene glycols, polyethylene glycols, glycerol triacetate, gelatine, carbohydrates, such as lactose, sucrose, mannitol, sorbitol or starch (maize starch, wheat starch, rice starch, potato starch), cellulose preparations and/or calcium phosphates, for example tricalcium phosphate or calcium hydrogen phosphate, magnesium stearate, talc, gelatine, tragacanth, methyl cellulose, hydroxypropylmethylcellulose, sodium carboxymethylcellulose, polyvinyl pyrrolidone and/or vaseline.

If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries include, without limitation, flow-regulating agents and lubricants, for example, silica, talc, stearic acid or salts thereof, such as magnesium stearate or calcium stearate, and/or polyethylene glycol. Dragee cores are provided with suitable coatings, which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices or to provide a dosage form affording the advantage of prolonged action, the tablet, dragee or pill can comprise an inner dosage and an outer dosage component the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol, solutions of suitable cellulose preparations such as acetyl-cellulose phthalate, cellulose acetate or hydroxypropylmethyl-cellulose phthalate, are used. Dye stuffs or pigments may be added to the tablets or dragee coatings, for example, for identification or in order to characterize combinations of active compound doses.

Suitable carrier substances are organic or inorganic substances which are suitable for enteral (e.g. oral) or parenteral administration or topical application and do not react with the novel compounds, for example water, vegetable oils, benzyl alcohols, polyethylene glycols, gelatin, carbohydrates such as lactose or starch, magnesium stearate, talc and petroleum jelly. In particular, tablets, coated tablets, capsules, syrups, suspensions, drops or suppositories are used for enteral administration, solutions, preferably oily or aqueous solutions, furthermore suspensions, emulsions or implants, are used for parenteral administration, and ointments, creams or powders are used for topical application. The compounds of the invention can also be lyophilized and the lyophilizates obtained can be used, for example, for the production of injection preparations.

Other pharmaceutical preparations, which can be used orally include push-fit capsules made of gelatine, as well as soft, sealed capsules made of gelatine and a plasticizer such as glycerol or sorbitol. The push-fit capsules can contain the active compounds in the form of granules, which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

The liquid forms in which the novel compositions of the present invention may be incorporated for administration orally include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatine.

Suitable formulations for parenteral administration include aqueous solutions of the active compounds in water-soluble form, for example, water-soluble salts and alkaline solutions. In addition, suspensions of the active compounds as appropriate oily injection suspensions may be administered.

Suitable lipophilic solvents or vehicles include fatty oils, for example, sesame oil, or synthetic fatty acid esters, for example, ethyl oleate or triglycerides or polyethylene glycol-400 (the compounds are soluble in PEG-400).

Aqueous injection suspensions may contain substances, which increase the viscosity of the suspension, including, for example, sodium carboxymethyl cellulose, sorbitol, and/or dextran, optionally, the suspension may also contain stabilizers.

For administration as an inhalation spray, it is possible to use sprays in which the active ingredient is either dissolved or suspended in a propellant gas or propellant gas mixture (for example CO₂ or chlorofluorocarbons). The active ingredient is advantageously used here in micronized form, in which case one or more additional physiologically acceptable solvents may be present, for example ethanol. Inhalation solutions can be administered with the aid of conventional inhalers.

Possible pharmaceutical preparations, which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatine rectal capsules, which consist of a combination of the active compounds with a base. Possible base materials include, for example, liquid triglycerides, polyethylene glycols, or paraffin hydrocarbons.

For use in medicine, the compounds of the present invention may be in the form of pharmaceutically acceptable salts. Other salts may, however, be useful in the preparation of the compounds of the invention or of their pharmaceutically acceptable salts. Suitable pharmaceutically acceptable salts of the compounds of this invention are those described hereinbefore and include acid addition salts which may, for example be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic bases, e.g. quaternary ammonium salts.

The pharmaceutical preparations can be employed as medicaments in human and veterinary medicine. As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Said therapeutic effective amount of one or more of the compounds of the invention is known to the skilled artisan or can be easily determined by standard methods known in the art.

The compounds of the present invention and the optional additional active substances are generally administered analogously to commercial preparations. Usually, suitable doses that are therapeutically effective lie in the range between 0.0005 mg and 1000 mg, preferably between 0.005 mg and 500 mg and especially between 0.5 mg and 100 mg per dose unit. The daily dose is preferably between about 0.001 mg/kg and 10 mg/kg of body weight.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The specific dose for the individual patient, in particular for the individual human patient, depends, however, on the multitude of factors, for example on the efficacy of the specific compounds employed, on the age, body weight, general state of health, the sex, the kind of diet, on the time and route of administration, on the excretion rate, the kind of administration and the dosage form to be administered, the pharmaceutical combination and severity of the particular disorder to which the therapy relates. The specific therapeutic effective dose for the individual patient can readily be determined by routine experimentation, for example by the doctor or physician, which advises or attends the therapeutic treatment.

The compounds of the present invention can be prepared according to the procedures of the following Schemes and Examples, using appropriate materials, and as further exemplified by the following specific examples. They may also be prepared by methods known per se, as described in the literature (for example in standard works, such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions which are known and suitable for the said reactions. Use can also be made of variants which are known per se, but are not mentioned here in greater detail.

Likewise, the starting materials for the preparation of compounds of the present invention can be prepared by methods as described in the examples or by methods known per se, as described in the literature of synthetic organic chemistry and known to the skilled person, or can be obtained commercially. The starting materials for the processes claimed and/or utilized may, if desired, also be formed in situ by not isolating them from the reaction mixture, but instead immediately converting them further into the compounds of the invention or intermediate compounds. On the other hand, in general it is possible to carry out the reaction stepwise.

Preferably, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, dimethylformamide (DMF) or N-methyl pyrrolidinone (NMP); nitriles, such as acetonitrile; sulfoxides, such as dimethyl sulfoxide (DMSO); nitro compounds, such as nitromethane or nitrobenzene; esters, such as ethyl acetate, or mixtures of the said solvents or mixtures with water.

The reaction temperature is between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring.

Based on the reaction temperatures given above, suitable reaction times generally lie in the range between 10 minutes and 48 hours.

Moreover, by utilizing the procedures described herein, in conjunction with ordinary skills in the art, additional compounds of the present invention claimed herein can be readily prepared. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The present invention also refers to a process for manufacturing a compound according to formula (I), or derivatives, N-oxides, prodrugs, solvates, tautomers or stereoisomers thereof as well as the physiologically acceptable salts of each of the foregoing. This process is characterized in that (a) a compound of formula (II)

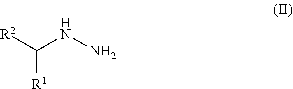

wherein
R¹ and R² are as defined hereinabove and in claim 1 for formulas (Ia), (Ib) and (Ic);
is reacted with a compound of formula (III)

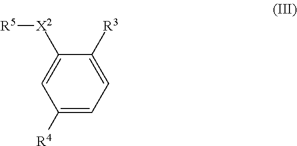

wherein
R³, R⁴ and X² are as defined hereinabove and in claim 1 for formulas (Ia), (Ib) and (Ic);
R⁵ denotes a malonic acid dialkyl ester residue, —CH—(C(O)—O—C₁₋₄alkyl)₂, or a malonic acid dihalide, —CH—(C(O)-Hal)₂ with Hal being Cl or Br;
to yield a compound of formulas (Ia) and/or (Ib) and/or (Ic)

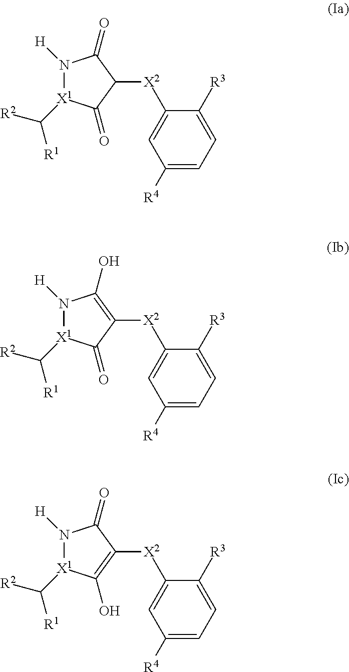

wherein
R¹, R², R³, R⁴ and X² are as defined hereinabove and in claim 1; and
X¹ denotes N;
or
(b) a compound of formula (IV)

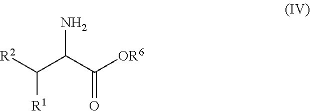

wherein
R¹ and R² are as defined hereinabove and in claim 1 for formulas (Ia), (Ib) and (Ic);
R⁶ denotes C₁₋₄-alkyl;
is reacted with a compound of formula (V)

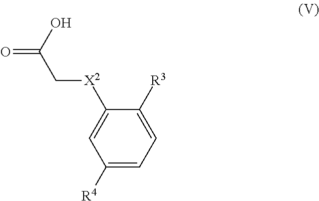

wherein
R³, R⁴ and X² are as defined hereinabove and in claim 1 for formulas (Ia), (Ib) and (Ic);
to first form an amide of formula (VI)

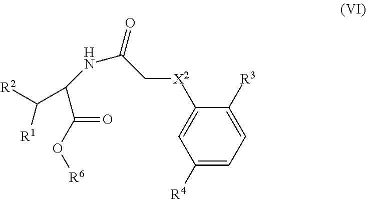

wherein
R¹, R², R³, R⁴, R⁶ and X² are as defined hereinabove and in claim 1;
and then convert that amide of formula (VI) into a compound of formulas (Ia)

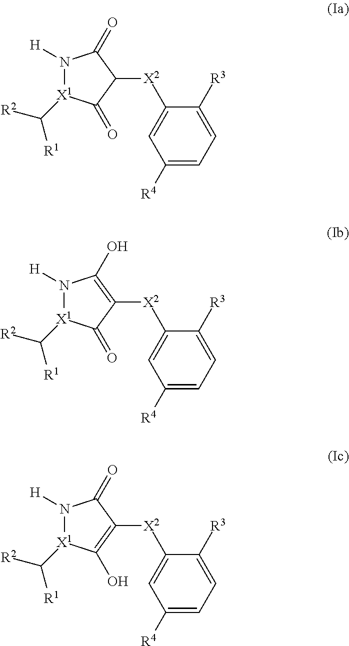

wherein
R¹, R², R³, R⁴ and X² are as defined hereinabove and in claim 1; and
X¹ denotes CH.

As will be understood by the person skilled in the art of organic synthesis compounds of the present invention, in particular compounds of formula (I), are readily accessible by various synthetic routes, some of which are exemplified in the accompanying Experimental Part. The skilled artisan will easily recognize which kind of reagents and reactions conditions are to be used and how they are to be applied and adapted in any particular instance—wherever necessary or useful—in order to obtain the compounds of the present invention. Furthermore, some of the compounds of the present invention can readily be synthesized by reacting other compounds of the present invention under suitable conditions, for instance, by converting one particular functional group being present in a compound of the present invention, or a suitable precursor molecule thereof, into another one by applying standard synthetic methods, like reduction, oxidation, addition or substitution reactions; those methods are well known to the skilled person.

Likewise, the skilled artisan will apply—whenever necessary or useful—synthetic protecting (or protective) groups; suitable protecting groups as well as methods for introducing and removing them are well-known to the person skilled in the art of chemical synthesis and are described, in more detail, in, e.g., P.G.M. Wuts, T.W. Greene, “Greene's Protective Groups in Organic Synthesis”, 4th edition (2006) (John Wiley & Sons).

A particularly versatile starting point for making compounds of formula (I) with X¹ being N are precursor (or intermediate) molecules PRE1 and PRE4. Compounds of formula PRE1 are readily available as shown in Scheme 1 below:

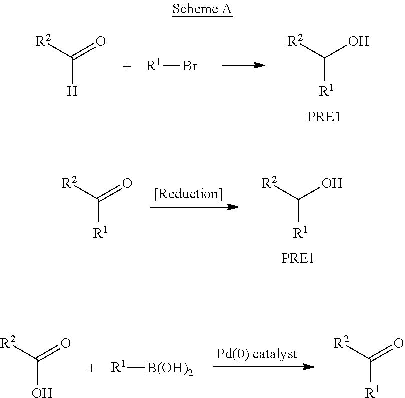

One starting point could be the aldehyde R²CHO which is reacted with the bromide R¹—Br under suitable reaction conditions to form the secondary alcohol, PRE1. Alternatively, the carbonyl compound R¹R²C(═O) (either being an aldehyde, if R¹ is H, or a ketone, if R¹ is a substituent not being hydrogen) can be transformed into the respective intermediate, PRE1, by applying a suitable reduction means, like, e.g., NaBH₄. Ketones R¹R²C(═O) are either commercially available or can be prepared by utilizing known synthetic methodologies one of which is depicted in Scheme A above: The carboxylic acid R²COOH is reacted with the boronic acid R¹—B(OH)₂ in the presence of a suitable organometallic Pd(0) transition metal complex under appropriate C—C coupling reaction conditions to form the respective ketone.

In subsequent reaction steps PRE1 may then be converted into the hydrazine precursor molecule, PRE3, as depicted in Scheme B below.

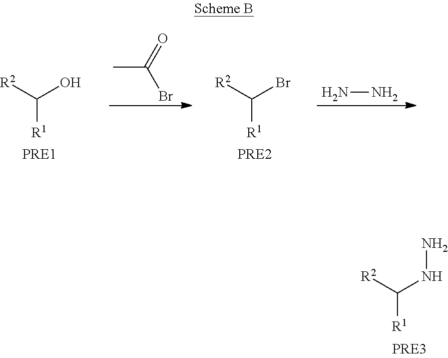

PRE1 is converted into the respective bromide, PRE2, by utilizing an appropriate bromination reagent, e.g., acetyl bromide; PRE2 in turn is reacted with hydrazine to form intermediate PRE3. PRE3, however, is identical to a compound of formula (II) as described hereinabove which is afterwards reacted with PRE4 (see below) which is identical to a compound of formula (III) as described hereinabove to form a compound of formula (I) with X¹ being N.

PRE4 (or compound of formula (III)) can be obtained by reacting a suitably 2-halogen substituted malonic dialkylester with a suitably substituted phenol or thiophenol, as shown in Scheme C below.

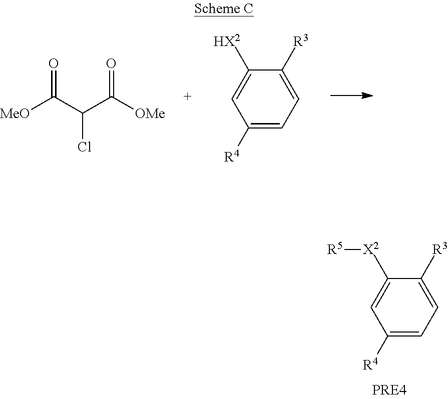

For instance, the dimethyl ester of chloro malonic acid may be reacted with an optionally substituted phenol or thiophenol in the presence of a base like triethylamine in order to form PRE4 with R⁵ being CH(C(O)—OCH₃)₂, X² being either S or O and R³ and R⁴ having the meaning as for compounds of formula (I).

A particularly versatile starting point for making compounds of formula (I) with
X¹ being CH are precursor (or intermediate) molecules PRE5 (i.e., compounds of formula (IV)) and PRE6 (i.e., compounds of formula (V)) which may be reacted with each other to first form the respective amide of formula (VI) which is then cyclized to form a compound of formula (I) with X¹ being CH.

Compounds of formula (IV) (═PRE5) are readily available by first reacting a suitably substituted compound PRE2 with ethyl 2-(benzhydrylideneamino)acetate in the presence of an aqueous sodiumhydroxide solution (50%) and tetrabutylaminobromide (TBAB) and subsequent hydrolysis and esterification according to the reaction sequence shown in Scheme D.

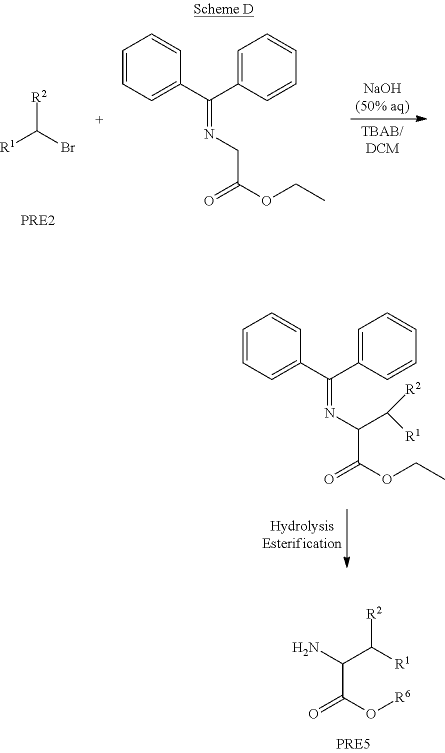

Compounds of formula (V) (═PRE6) are readily available according the reaction shown in Scheme E.

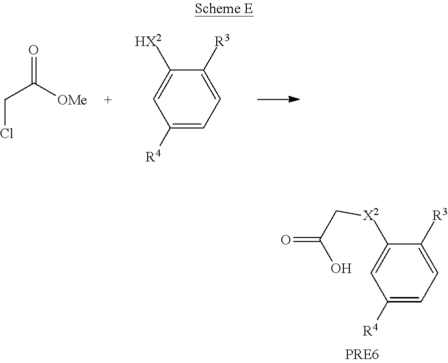

An alkyl ester of 2-halogen substituted acetic acid, e.g., methyl chloroacetate or ethyl bromoacetate, may be reacted with a suitably substituted phenol or thiophenol in the presence of a base, e.g., sodium hydroxide, to form PRE6.

In a variant of that synthetic route instead of an alkyl ester of 2-halogen substituted acetic acid dialkyl esters of 2-chloromalonic acid may be used in the presence of a base like trimethylamine, followed by the addition of NaHCO₃ and KHSO₄, which yields PRE6 in an nucleophilic substitution and decarboxylation reaction sequence.

Compounds of the present invention according to formulas (Ia), (Ib) and/or (Ic) can be further modified in order to afford further compounds of formulas (Ia), (Ib) and/or (Ic) that are structurally modified. For instance, compounds of formulas (Ia), (Ib) and/or (Ic) bearing a carboxylic ester group at one of the substituents R¹ or R² may be converted into the respective amides or hydrazides by reacting the ester with suitable reagents. Furthermore, for instance, compounds of the present invention of formula (I) with R² being, e.g., an (optionally substituted) aryl or hetereoaryl substituent, i.e., Ar^{X} or Hetar^{X}, may be transformed into other compounds of the present invention of formula (I) with more complex substituents R², e.g., Ar^{X}—Ar^{Y}, Ar^{X}-Hetar^{Y}, Ar^{X-}Hetcyc^{Y}, Hetar^{X}-Ar^{Y}, Hetar^{X}-Hetar^{Y}, Hetar^{X}-Hetcyc^{Y}, by utilizing well-known C—C and C—N coupling reactions.

Typical suitable C—C coupling reactions are, among others, the Heck reaction, the Suzuki coupling, the Stille coupling, the Negishi coupling and coupling reactions utilizing organo cuprates, and well-known variants thereof.

Depending on the specific method applied reagents, solvents and reaction conditions are selected accordingly. For instance, a compound of formula (I) with R² being a halide substituted moiety Ar^{X} or Hetar^{X} may be subjected to typical conditions of a Suzuki coupling reaction, thereby reacting the halide with a suitable borate or boronate ester, (B(OSub)₃ with Sub being a suitable substituent, radical or residue (like trimethylborate or 4,4,5,5-tetramethyl-2-(tetramethyl-1,3,2-dioxaborolan-2-yl)-1,3,2-dioxaborolane) in the presence of an organometallic palladium (II) catalyst (like [1,1′-bis(diphenyl)phosphino)-ferrocene]-dichloropalladium(II) dichloromethane complex) and optionally potassium acetate in order to form an intermediate compound in which the halogen substituent of Ar^{X} or Hetar^{X} is replaced by —B(OH)₂ or —B(OSub)₂, as the case may be; this intermediate compound may then be reacted with a suitable halide, e.g., Ar^{Y}-Hal or Hetar^{Y}-Hal or Hetcyc^{Y}-Hal in the presence of a palladium(0) complex (e.g., tetrakis(triphenylphosphine)palladium(0)) and a base (e.g., sodium, potassium or cesium carbonate) to build a compound of formula (I). Similarly, the same compound of formula (I) can be obtained by forming a boron-substituted precursor Ar^{Y}—B(OH)₂, Hetar^{Y}-B(OH)₂, Hetcyc^{Y-}B(OH)₂ or Ar^{Y}—B(OSub)₂, Hetar^{Y}-B(OSub)₂, Hetcyc^{Y}-B(OSub)₂ and reacting it with the halide substituted starting compound of formula (I) under similar conditions.

Likewise, C—N coupling reactions may be any suitable C—N coupling reaction of a heterocyclic system or a molecule bearing a reactive amino group with a suitably substituted compound of formula (I). Depending on the specific coupling reaction applied, it may well be that one or both of the reaction partners are subject to chemical transformation into intermediates before the reaction with the appropriate reaction partner occurs; for instance, the suitably substituted halide may be transformed into a respective boronic acid or boronic acid ester derivative before the reaction with the heterocyclic system or the reactive amine derivative occurs. Preferably, this coupling reaction is performed in the presence of a transition metal catalyst. Well-known examples of such C—N coupling reactions are, among others, the Hartwig-Buchwald reaction, the Ullmann coupling reaction, reactions similar to Suzuki or Heck reaction and coupling reactions utilizing organo cuprates. Depending on the specific method applied reagents, solvents and reaction conditions are selected accordingly.

It goes without saying that any of these C—C and C—N coupling reactions may also be utilized to introduce more complex substituents R¹ or R² on the stage of the prescursor molecules PRE1 by modifying different, structurally less complex substituents R¹ or R² rather than on the stage of a compound of formula (I).

### EXPERIMENTAL PART

### Abbreviations

[(Cinnamyl)PdCl]₂—Palladium(π-cinnamyl) chloride dimer
BINAP—(±)-2,2′-Bis(diphenylphosphino)-1,1′-binaphthalene
BippyPhos—5-(Di-tert-butylphosphino)-1′,3′,5′-triphenyl-1′H-[1,4′]bipyrazole
BrettPhos—2-(Dicyclohexylphosphino)3,6-dimethoxy-2′,4′,6′-triisopropyl-1,1′-biphenyl
BrettPhos precatalyst—Chloro[2-(dicyclohexylphosphino)-3,6-dimethoxy-2′,4′,
6′-triisopropyl-1,1′-biphenyl][₂—(2-aminoethyl)phenyl]palladium(II)
t-BuBrettPhos—2-(Di-tert-butylphosphino)-2′,4′,6′-triisopropyl-3,6-dimethoxy-1,1′-biphenyl
t-BuOH—2-Methylpropan-2-ol
tBuXPhos—2-Di-tert-butylphosphino-2′,4′,6′-triisopropylbiphenyl
CH₃COOH—Acetic acid
DCM—Dichloromethane
DIPEA—Ethyldiisopropylamine
DME—1,2-Dimethoxyethane
DMF—N,N-Dimethylformamide
DMSO—Dimethyl sulfoxide
EtOAc—Ethyl acetate
EtOH—Ethanol
Et₂O—Diethyl ether
Hantzsch ester—Diethyl 1,4-dihydro-2,6-dimethyl-3,5-pyridinedicarboxylate
HCl—Hydrochloric acid solution
Herrmann's catalyst—trans-Bis(acetato)bis[o-(di-o-tolylphosphino)benzyl]dipalladium(II)
HPLC—High-performance liquid chromatography
KOAc—Potassium acetate
LiHMDS—Lithium bis(trimethylsilyl)amide solution
Me₄tBuXPhos—2-Di-tert-butylphosphino-3,4,5,6-tetramethyl-2′,4′,6′-triisopropyl-1,1′-biphenyl
MeOH—Methanol
MgSO₄—Magnesium sulfate
MW—Microwave
Na₂CO₃— Sodium carbonate
NaOAc—Sodium acetate
NaBH(OAc)₃—Sodium triacetoxyborohydride
NaOtBu—Sodium tert-butoxide
Pd(dppf)C₂ —[1,1′-Bis(diphenylphosphino)ferrocene]dichloropalladium(II)
Pd(dppf)Cl₂.CH₂Cl₂-[1,1′-Bis(diphenylphosphino)ferrocene]dichloropalladium(II), complex with dichloromethane
Pd(OAc)₂—Palladium(II) acetate
Pd(PPh₃)₄—Tetrakis(triphenylphosphine)palladium(0)
Pd₂(dba)₃—Tris(dibenzylideneacetone)dipalladium(0)
PTSA—p-Toluenesulfonic acid monohydrate
RM—reaction mixture
rt—room temperature
RT—Retention time
t-BuBrettPhos—2-(Di-tert-butylphosphino)-2′,4′,6′-triisopropyl-3,6-dimethoxy-1,1′-biphenyl
TEA—Triethylamine
TEA*HCl—Triethylamine hydrochloride
TFA—Trifluoroacetic acid
THF—Tetrahydrofuran
TMCS—Chlorotrimethylsilane
TTIP—Titanium(IV) isopropoxide
Xantphos—4,5-Bis(diphenylphosphino)-9,9-dimethylxanthene
Xphos—2-Dicyclohexylphosphino-2′,4′,6′-triisopropylbiphenyl

The compounds of the present invention can be prepared according to the procedures of the following Schemes and Examples, using appropriate materials and are further exemplified by the following specific examples. Analytical data of compounds made according to the following examples are shown in the accompanying tables.

The invention will be illustrated, but not limited, by reference to the specific embodiments described in the following examples. Unless otherwise indicated in the schemes, the variables have the same meaning as described above and in the claims.

Unless otherwise specified, all starting materials are obtained from commercial suppliers and used without further purifications. Unless otherwise specified, all temperatures are expressed in ° C. and all reactions are conducted at RT. Compounds are purified by either silica chromatography or preparative HPLC.

¹H NMR is recorded on 400 MHz spectrometers. Chemical shifts (6) are reported in ppm relative to the residual solvent signal (6=2.5 ppm for 1H NMR in DMSO-d6). ¹H NMR data are reported as follows: chemical shift (multiplicity, coupling constants and number of hydrogens). Multiplicity is abbreviated as follows: s (singlet), d (doublet), t (triplet), q (quartet), m (multiplet), br (broad).

### LC-MS Analyses

### Method 1:

Equipment: Shimadzu LC-MS 2020 column: Waters Acquity UPLC HSS C18,
50 mm×2.1 mm×1.8 μm

### Eluents:

(A) 0.1% formic acid in ACN
(B) 0.1% formic acid in water
Autosampler: injection volume: 1 μL

### Pump:

| Time | Flow | % |
|---|---|---|
| [min] | [mL/min] | B |
| 0.00 | 0.5 | 95 |
| 0.00 | 0.5 | 95 |
| 4.00 | 0.5 | 5 |
| 5.00 | 0.5 | 5 |
| 5.20 | 0.5 | 95 |
| 6.00 | 0.5 | 95 |

Column compartment:

column temperature: 25° C.

time of analysis: 6 min

### Detector:

wavelengths: 254, 230, 270, 280 nm

### Method 2:

Equipment: MS Bruker Amazon SL; LC Dionex Ultimate 3000; HPLC with UV-Vis or DAD detector
column: Kinetex XB C18 4.6×50 mm 2.6 μm

### Eluents:

(A) 0.1% formic acid-water solution
(B) 0.1% formic acid-ACN solution
Autosampler: injection volume: 1 μL
Pump: flow: 0.5 mL/min

| Time | [%] |
|---|---|
| [min] | B |
| 0.0 | 20 |
| 6.7 | 80 |
| 7.5 | 80 |
| 7.8 | 95 |
| 9.5 | 95 |
| 10.0 | 20 |
| 12.0 | 20 |

Column compartment:

column temperature: 25° C.

time of analysis: 12 min

### Detector:

wave length: 220, 254, 280 nm

### Method 3:

Equipment: MS Bruker Amazon SL; LC Dionex Ultimate 3000; HPLC with UV-Vis or DAD detector

### Column: Waters Symmetry C18 3.9×150 mm 5 μm

### Eluents:

(A) 0.1% formic acid-water solution
(B) 0.1% formic acid-ACN solution
Autosampler: injection volume: 3 μL
Pump:—flow: 1.2 mL/min

| Time | [%] |
|---|---|
| [min] | B |
| 0.0 | 20 |
| 20.0 | 80 |
| 22.0 | 80 |
| 22.5 | 95 |
| 25.0 | 95 |
| 25.3 | 20 |
| 30.0 | 20 |

Column compartment:

column temperature: 25° C.

time of analysis: 30 min

### Detector:

wave length: 254 nm

### Method 4:

Equipment: MS Bruker Amazon SL, LC Dionex Ultimate 3000, HPLC with UV-Vis or DAD detector
column: ACE C18-AR 100 A 250×4.60 mm 5 μm

### Eluents:

(A) ammonium formate buffer c=20 mM pH=8
Weighed on analytical balance 1,2612 g of ammonium formate, placed in 1 L volumetric flask and dissolved in 800 mL of distilled water. Adjusted to pH=8.0 using ammonium hydroxide (25% solution). Diluted to the mark using distilled water.

### (B) ACN

Autosampler: injection volume: 3 μL
Pump:—flow: 1.2 mL/min

| Time | [%] | [%] |
|---|---|---|
| [min] | A | B |
| 0.0 | 80 | 20 |
| 20.0 | 20 | 80 |
| 22.0 | 20 | 80 |
| 22.5 | 5 | 95 |
| 25.0 | 5 | 95 |
| 26.0 | 80 | 20 |
| 30.0 | 80 | 20 |

Column compartment:

column temperature: 25° C.

time of analysis: 30 min

### Detector:

wave length: 214, 254 nm

### Method 5:

Equipment: MS Bruker Amazon SL, LC Dionex Ultimate 3000, HPLC with UV-Vis or DAD detector

column: Waters Symmetry C18 3.9×150 mm 5 μm

### Eluents:

(A) 0.1% formic acid-water solution

(B) 0.1% formic acid-ACN solution

Analytical method:
Autosampler:—injection volume: 3 μL
Pump:—flow: 1.0 ml/min

| Time | [%] | [%] |
|---|---|---|
| [min] | A | B |
| 0.0 | 95 | 5 |
| 5.0 | 95 | 5 |
| 25.0 | 20 | 80 |
| 27.0 | 20 | 80 |
| 28.0 | 95 | 5 |
| 30.0 | 95 | 5 |

Column compartment:

column temperature: 25° C.

time of analysis: 30 min

### Example 1A

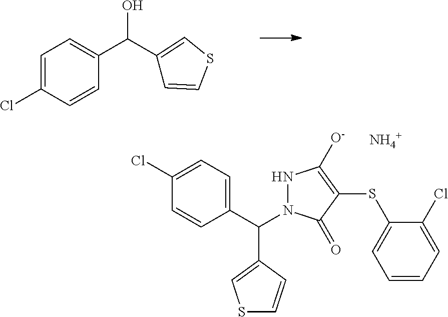 [SMILES: OC(c1ccc(Cl)cc1)c1ccsc1 ; O=c1c(Sc2ccccc2Cl)c([O-])[nH]n1C(c1ccc(Cl)cc1)c1ccsc1 ; [NH4+]]

### Ammonium 1-[(4-chlorophenyl) (thiophen-3-yl)methyl]-4-[(2-chlorophenyl)sulfanyl]-5-oxo-2,5-dihydro-1H-pyrazol-3-olate

(4-Chlorophenyl)-thiophen-3-yl-methanol (Intermediate 3A) (3.38 g; 13.38 mmol; 1.00 eq.) is refluxed with acetyl bromide (1.98 ml; 26.76 mmol; 2.00 eq.) for 30 min, then RM is cooled to rt and evaporated under reduced pressure to give crude 3-[bromo-(4-chlorophenyl)-methyl]-thiophene (4.00 g; 9.74 mmol; 72.8%; brown oil) which is immediately used in the next step. Method 1: RT 3.63 min, p 85%, (M-Br)+207.0.

The solution of crude 3-[bromo-(4-chlorophenyl)-methyl]-thiophene (2.00 g; 4.87 mmol; 1.00 eq.) from previous step in 5 ml of dry THF is added dropwise to the 1M hydrazine solution in THF (19.47 ml; 19.47 mmol; 4.00 eq.) for 20 min. The reaction mixture is stirred overnight at rt and subsequently refluxed for additional 30 min. After that reaction mixture is cooled, diluted with diethyl ether and washed 3 times with water to remove the excess of hydrazine. The organic layer is then extracted with 0.2 M HCl. Then the water extract is basified with 2 M NaOH and extracted with diethyl ether. The organic layer is washed with brine, dried over MgSO₄ and evaporated to give crude [(4-chlorophenyl)(thiophen-3-yl)methyl]hydrazine (1.17 g; 3.93 mmol; yield: 81%; brown oil) which is directly used in next step. Method 1: RT 2.29 min, p 83%, (M-NHNH₂)⁺207.0.

[(4-Chlorophenyl)-thiophen-3-yl-methyl]-hydrazine (1.17 g; 4.91 mmol; 1.00 eq.) obtained in the previous step and 2-(2-chlorophenylsulfanyl)-malonic acid dimethyl ester (1.67 g; 5.90 mmol; 1.20 eq.) are dissolved in [1,4]-dioxane (12.00 ml) and heated at 160° C. for 30 min. Then RM is diluted with AcOEt and hexane (1:1) and extracted with 2% aq. ammonia. The aqueous extract is washed with mixture of AcOEt/Hexane, evaporated and purified with reversed phase (C-18) flash chromatography using 0.1% NH₃ in water: ACN (0-100%) to give ammonium 1-[(4-chlorophenyl)(thiophen-3-yl)methyl]-4-[(2-chlorophenyl)sulfanyl]-5-oxo-2,5-dihydro-1H-pyrazol-3-olate (0.75 g; 1.62 mmol; 33%); Method 1: RT 3.34 min, p 100%, M+H 448.9, M−H 446.8. Method 3: RT 17.7 min, p 97.5%, M+1 448.7; ¹H NMR (400 MHz, D20) δ 7.41 (dd, J=5.0, 3.0 Hz, 1H), 7.38-7.27 (m, 5H), 7.26-7.18 (m, 2H), 7.07 (dd, J=5.1, 1.2 Hz, 1H), 7.00-6.89 (m, 3H), 6.31 (s, 1H), 6.13 (dd, J=6.0, 3.4 Hz, 1H), 1.22 (d, J=6.8 Hz, 1H).

The following compounds are prepared by the procedure for Example 1A, using the appropriate starting materials:

| | | | | | Starting |
|---|---|---|---|---|---|
| | | | | | materials |
| Ex. | | | | | (SM) and |
| No. | Name and structure | NMR | mz | RT | yield |
| 1B | 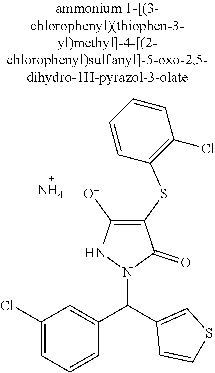 | ¹H NMR (400 MHz, DMSO/D2O) δ: 9.64 (dd, J = 5.0, 3.0 Hz, 1H), 9.57- 9.50 (m, 2H), 9.48-9.41 (m, 4H), 9.21 (dd, J = 5.0, 1.2 Hz, 1H), 9.19-9.10 (m, 2H), 8.51 (s, 1H), 8.48-8.42 (m, 1H). | M + 1 448.9, M − 1 446.8. | Method 1: 3.39 min, p 99%, | Intermediate 3B, Yield: 1.125 g; 2.4 mmol, 28%. |
| 1C | 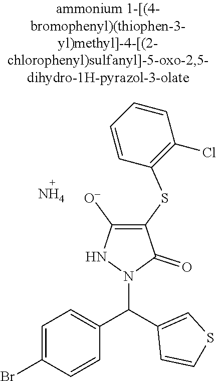 | ¹H NMR (400 MHz, DMSO) δ: 11.93 (s, 1H), 10.40 (s, 1H), 7.65-7.54 (m, 3H), 7.55 ż 7.45 (m, 1H), 7.39 (dd, J = 7.8, 1.3 Hz, 1H), 7.36-7.28 (m, 1H), 7.29-7.16 (m, 3H), 7.11 (td, J = 7.6, 1.6 Hz, 1H), 7.05 (dd, J = 5.0, 1.3 Hz, 1H), 6.66 (s, 1H). | M − 1 492.9 | Method 1: RT 3.46 min p 91%, | Intermediate 2A; Yield: 2.67 g; 4.9 mmol, 76%. |
| 1D | 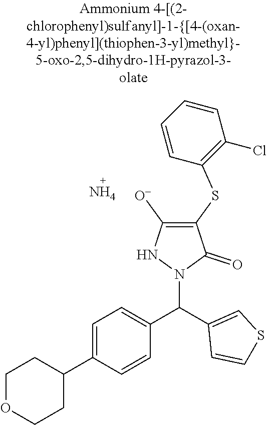 | 1H NMR (400 MHz, D₂O) δ 7.43 (dd, J = 5.0, 3.0 Hz, 1H), 7.34 (d, J = 8.3 Hz, 2H), 7.30 (d, J = 8.4 Hz, 2H), 7.27-7.22 (m, 2H), 7.08 (dd, J = 5.0, 1.2 Hz, 1H), 6.99-6.94 (m, 2H), 6.37 (s, 1H), 6.33-6.29 (m, 1H), 4.00 (dd, J = 11.8, 3.4 Hz, 2H), 3.55 (td, J = 11.4, 3.1 Hz, 2H), 2.89-2.81 (m, 1H), 1.80- 1.68 (m, 4H). | M − 1 487.1 | Method 3: RT 4.5 min, | Intermediate 3E; Yield: 7.20 mg; 0.01 mmol; 2.0%; white amorphous solid. |
| 1E | 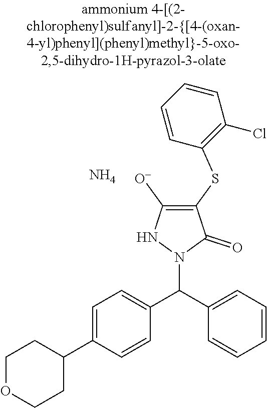 | ¹H NMR (400 MHz, CD₃OD) δ 7.41-7.34 (m, 5H), 7.33-7.27 (m, 4H), 7.24 (dd, J = 8.3, 4.2 Hz, 1H), 7.02-6.96 (m, 2H), 6.67-6.61 (m, 2H), 4.11- 4.02 (m, 2H), 3.64- 3.53 (m, 2H), 2.91-2.80 (m, 1H), 1.89-1.76 (m, 4H). | M − 1 491.4, M + 1 493.1 | Method 3: RT 14.6 min, p 100%, | Intermediate 3F; Yield: 106 mg, 0.21 mmol, 34%, white solid. |
| 1F | 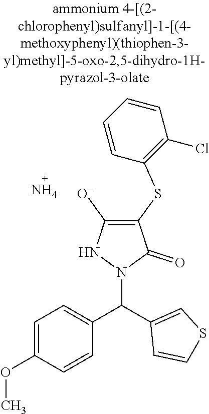 | not determined | M + 1 445.0, M − 1 443.0. | Method 3: RT 14.1 min, p 100%, | Intermediate 3C; Yield: 3.7 mg, 0.01 mmol, 0.6%. |
| 1G | 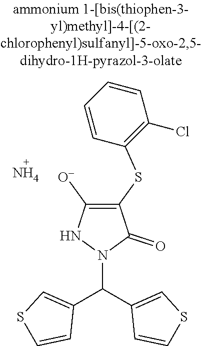 | ¹H NMR (400 MHz, D₂O) δ 7.42 (dd, J = 5.0, 3.0 Hz, 1H), 7.30 (dd, J = 1.8, 1.1 Hz, 1 H), 7.23 (dd, J = 7.6, 1.6 Hz, 1H), 7.15 (dd, J = 5.0, 1.2 Hz, 1H), 6.98 (dqd, J = 14.7, 7.4, 1.7 Hz, 1H), 6.39 (s, 1H), 6.18 (dd, J = 7.7, 1.8 Hz, 1H). | M + 1 420.85, M − 1 418.85. | Method 3: RT 13.5 min | Intermediate 3D; Yield: 60 mg, 0.14 mmol, 23%. |
| 1H | 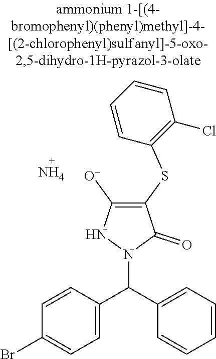 | 1H NMR (400 MHz, D2O) δ 7.56-7.50 (m, 2H), 7.39-7.30 (m, 5H), 7.26- 7.21 (m, 3H), 7.00- 6.93 (m, 2H), 6.34 (s, 1H), 6.25-6.20 (m, 1H). | M − 1 486.9, M + 1 489.0) | Method 1: RT 3.54 | Intermediate 3G; (1 g; 1.98 mmol; yield 22.3%) as a white amorphous solid |
| 1I | 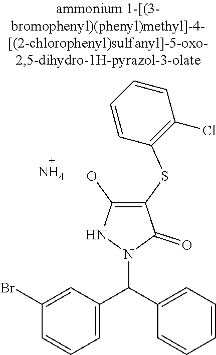 | 1H NMR (400 MHz, MeOD) δ 7.54-7.49 (m, 2H), 7.45-7.28 (m, 7H), 7.25-7.21 (m, 1H), 6.99 (dqd, J = 14.7, 7.4, 1.7 Hz, 2H), 6.62-6.58 (m, 2H). | M + 1 489.0. | Method 1: RT 4.38 | Intermediate 3H; Yield: 1.25 g; 2.44 mmol; 35% |

### Example 3A

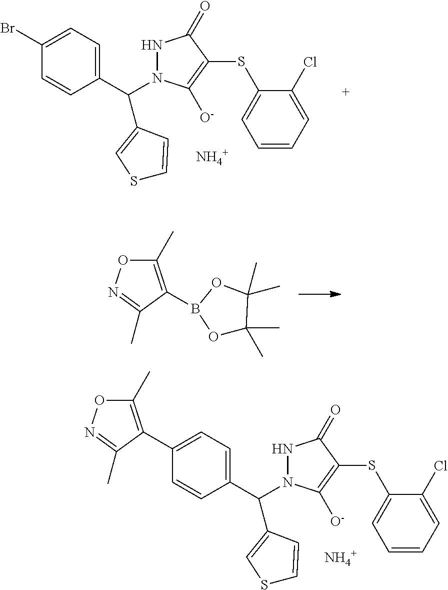 [SMILES: O=c1[nH]n(C(c2ccc(Br)cc2)c2ccsc2)c([O-])c1Sc1ccccc1Cl ; [NH4+] ; Cc1noc(C)c1B1OC(C)(C)C(C)(C)O1 ; Cc1noc(C)c1-c1ccc(C(c2ccsc2)n2[nH]c(=O)c(Sc3ccccc3Cl)c2[O-])cc1 ; [NH4+]]

### Ammonium 4-[(2-chlorophenyl)sulfanyl]-2-{[4-(dimethyl-1,2-oxazol-4-yl)phenyl](thiophen-3-yl)methyl}-5-oxo-2,5-dihydro-1H-pyrazol-3-olate

1-[(4-Bromophenyl)-thiophen-3-yl-methyl]-4-(2-chlorophenylsulfanyl)-pyrazolidine-3,5-dione (Example 1C) (0.10 g; 0.18 mmol; 1.00 eq.), cesium carbonate (180.12 mg; 0.55 mmol; 3.00 eq.), 3,5-dimethyl-4-(tetramethyl-1,3,2-dioxaborolan-2-yl)-1,2-oxazole (82.22 mg; 0.37 mmol; 2.00 eq.) are suspended in the mixture of 1,4-Dioxane (1.50 ml) and water (0.50 ml). The resulted mixture is purged with argon and Pd(dppf)Cl₂ (17.00 mg; 0.02 mmol; 0.13 eq.) is added, again purged with argon and heated in MW conditions at 120° C. for 30 min. Then RM is diluted with AcOEt, filtered through a pad of celite, washed with AcOEt. The filtrate is diluted with hexane (1:1) and extracted with 1% water ammonia. The water extract is evaporated and the residue is purified by prep HPLC (C-18, Water/0.01% NH3-ACN/0.01% NH3) to give ammonium 4-(2-Chloro-phenylsulfanyl)-1-{[4-(3,5-dimethyl-isoxazol-4-yl)-phenyl]-thiophen-3-yl-methyl}-5-hydroxy-1,2-dihydro-pyrazol-3-one (40.00 mg; 0.08 mmol; 41%; cream amorphous solid). Method 3: 15.9 min, p 100%, M−1 510.0. ¹H NMR (400 MHz, D20) δ 7.51-7.44 (m, 3H), 7.37 (d, J=8.3 Hz, 2H), 7.31 (d, J=2.9 Hz, 1H), 7.26-7.22 (m, 1H), 7.15-7.11 (m, 1H), 6.97-6.91 (m, 1H), 6.87 (t, J=7.0 Hz, 1H), 6.42 (s, 1H), 6.24 (dd, J=7.9, 1.5 Hz, 1H), 2.33 (s, 3H), 2.19 (s, 3H).

The following compounds are prepared by the procedure for Example 3A, using the appropriate starting materials:

| | | | | | Starting |
|---|---|---|---|---|---|
| Ex. | | | | | materials and |
| No. | Name and structure | NMR | mz | RT | yield |
| 3B | 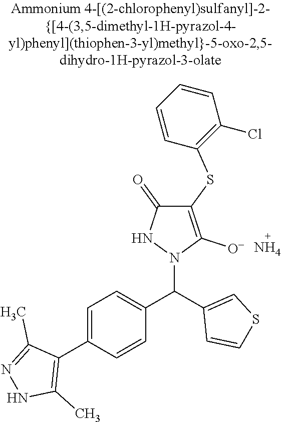 | ¹H NMR (400 MHz, D₂O) δ 7.49-7.43 (m, 3H), 7.35 (d, J = 8.2 Hz, 2H), 7.31 (d, J = 2.8 Hz, 1H), 7.23 (d, J = 7.9 Hz, 1H), 7.14 (d, J = 4.9 Hz, 1H), 6.92 (t, J = 6.8 Hz, 1H), 6.83 (t, J = 7.5 Hz, 1H), 6.40 (s, 1H), 6.20 (d, J = 7.9 Hz, 1H), 2.19 (s, 6H). | M + 1 509.2 | Method 3: RT 8.6 min, p 97.5%, | Example 1C and 3,5-dimethyl-4- (tetramethyl-1,3,2- dioxaborolan-2- yl)-1H- Yield: 25.40 mg; 0.05 mmol; 26%; white amorphous solid pyrazole |
| 3C | 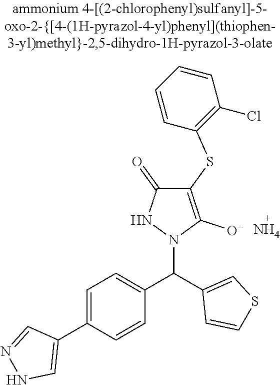 | ¹H NMR (400 MHz, D₂O) δ 8.03 (s, 2H), 7.61 (d, J = 8.3 Hz, 2H), 7.48- 7.39 (m, 3H), 7.30 (d, J = 2.8 Hz, 1H), 7.25- 7.20 (m, 1H), 7.13 (dd, J = 5.0, 1.1 Hz, 1H), 6.90 (dd, J = 11.5, 3.8 Hz, 1H), 6.82 (t, J = 7.3 Hz, 1H), 6.36 (s, 1H), 6.15 (d, J = 7.9 Hz, 1H). | M − 1 479.0. | Method 5: : RT 19.7 min | Example 1C and 4-(tetramethyl- 1,3,2- dioxaborolan-2- yl)-1H-pyrazole Yield: 21.00 mg; 0.04 mmol; 22%; amorphous solid. |
| 3D | 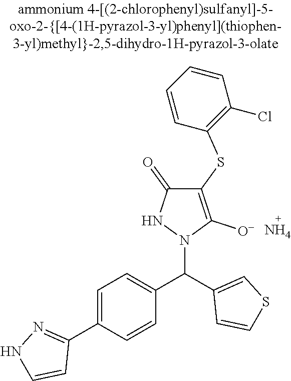 | ¹H NMR (400 MHz, D2O) δ 7.77 (d, J = 8.3 Hz, 2H), 7.73 (d, J = 2.3 Hz, 1H), 7.52-7.43 (m, 3H), 7.32 (s, 1H), 7.22 (d, J = 7.9 Hz, 1H), 7.14 (d, J = 3.9 Hz, 1H), 6.90 (t, J = 7.7 Hz, 1H), 6.81 (t, J = 7.7 Hz, 1H), 6.75 (d, J = 2.3 Hz, 1H), 6.39 (s, 1H), 3D6.13 (d, J = 8.0 Hz, 1H). | M − 1 479.0. | Method 5: RT 20.7 min | Example 1C and 3-(tetramethyl- 1,3,2- dioxaborolan-2- yl)-1H-pyrazole Yield: 12.00 mg; 0.02 mmol; 13%; white amorphous solid. |
| 3E | 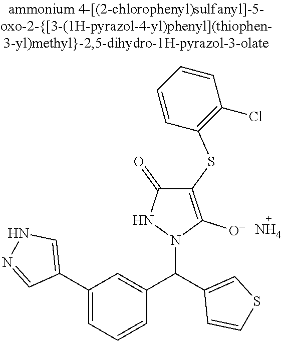 | | M − 1 479.0, M + 1 480.9 | Method 4: RT 7.1 min | Example 1B and 4-(tetramethyl- 1,3,2- dioxaborolan-2- yl)-1H-pyrazole Yield: 2.00 mg; 0.01 mmol; 1.8%; white amorphous solid |
| 3F | 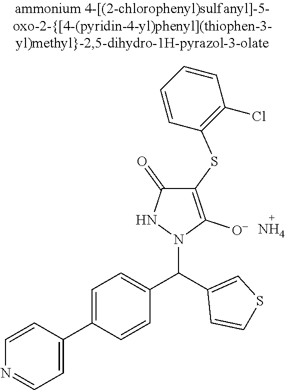 | ¹H NMR (400 MHz, D2O) δ 8.55-8.52 (m, 2H), 7.82-7.78 (m, 4H), 7.57- 7.52 (m, 2H), 7.46 (dd, J = 5.0, 2.9 Hz, 1H), 7.33-7.30 (m, 1H), 7.24-7.21 (m, 1H), 7.15- 7.12 (m, 1H), 6.90 (dd, J = 4.6, 3.3 Hz, 1H), 6.83-6.78 (m, 1H), 6.44-6.42 (m, 1H), 6.20- 6.17 (m, 1H). | M + 1 492.2 | Method 3: RT 4.2 min, p 98.7%, | Example 1C and (pyridin-4- yl)boronic acid Yield: 3.00 mg; 0.02 mmol; 13%; white amorphous solid. |
| 3G | 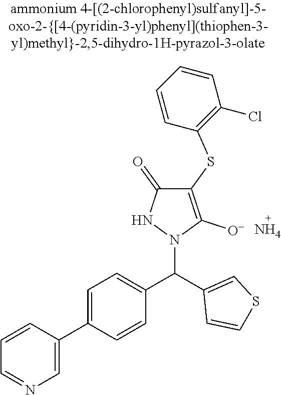 | ¹H NMR (400 MHz, D₂O) δ 8.75 (d, J = 1.7 Hz, 1H), 8.44 (dd, J = 4.9, 1.5 Hz, 1H), 8.10- 8.04 (m, 1H), 7.67 (d, J = 8.4 Hz, 2H), 7.48 (ddd, J = 9.7, 7.9, 5.6 Hz, 4H), 7.30 (d, J = 2.9 Hz, 1H), 7.22 (dd, J = 7.9, 1.2 Hz, 1H), 7.13 (dd, J = 5.1, 1.3 Hz, 1H), 6.96- 6.86 (m, 1H), 6.81 (t, J = 7.1 Hz, 1H), 6.43 (s, 1H), 6.21 (dd, J = 7.9, 1.5 Hz, 1H). | M + 1 492.5 | Method 3: RT 5.2 min | Example 1C and (pyridin-3- yl)boronic acid Yield: 33.70 mg; 0.07 mmol; 36%; white amorphous solid |
| 3H | 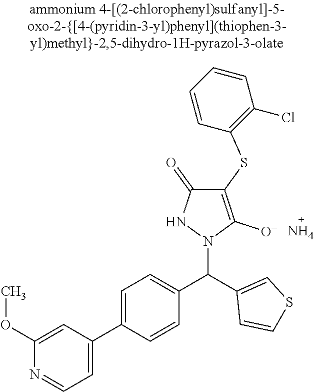 | ¹H NMR (400 MHz, D₂O) δ 8.10 (d, J = 5.4 Hz, 1H), 7.74 (d, J = 8.1 Hz, 2H), 7.52 (d, J = 8.2 Hz, 2H), 7.46 (s, 1H), 7.32 (d, J = 6.0 Hz, 2H), 7.22 (d, J = 7.9 Hz, 1H), 7.14 (d, J = 6.3 Hz, 2H), 6.90 (t, J = 7.7 Hz, 1H), 6.80 (t, J = 7.3 Hz, 1H), 6.42 (s, 1H), 6.17 (d, J = 8.0 Hz, 1H), 3.89 (s, 3H). | M + 1 522.1 | Method 3: RT 15.7 min | Example 1C and (2- methoxypyridin-4- yl)boronic acid Yield: 15.90 mg; 0.03 mmol; 16%; white amorphous solid. |
| 3I | 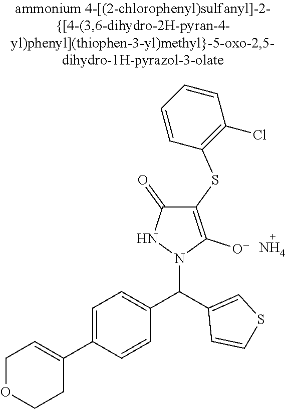 | ¹H NMR (400 MHz, DMSO/D₂O) δ 7.53-7.19 (m, 7H), 6.98 (br m, 3H), 6.67 (br s, 1H), 6.41 (br d, J = 26.3 Hz, 1H), 6.29-6.23 (m, 1H), 4.21 (dd, J = 5.0, 2.3 Hz, 2H), 3.81 (t, J = 5.5 Hz, 2H), 2.47-2.40 (m, 2H). | M − 1 495.0. | Method 3: RT 15.0 min | Example 1C and 2-(3,6-dihydro-2H- pyran-4-yl)- 4,4,5,5- tetramethyl-1,3,2- dioxaborolane Yield: 8.20 mg; 0.01 mmol; 8.1%; white amorphous solid |
| 3J | 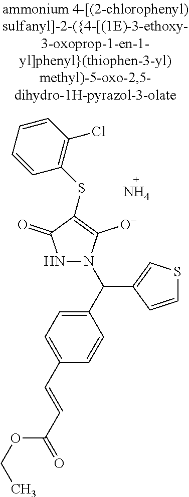 | 1H NMR (400 MHz, D2O) δ 7.72 (d, J = 16.1 Hz, 1H), 7.61 (d, J = 8.3 Hz, 2H), 7.48-7.40 (m, 3H), 7.28 (d, J = 2.8 Hz, 1H), 7.23 (dd, J = 7.9, 1.2 Hz, 1H), 7.11 (dd, J = 5.0, 1.2 Hz, 1H), 6.99-6.91 (m, 1H), 6.88 (t, J = 7.0 Hz, 1H), 6.53 (d, J = 16.1 Hz, 1H), 6.38 (s, 1H), 6.17 (dd, J = 7.9, 1.4 Hz, 1H), 4.20 (q, J = 7.1 Hz, 2H), 1.25 (t, J = 7.1 Hz, 3H). | M + 1 513.1, M − 1 511.8. | Method 3: RT 17.8 min | Example 1C and ethyl (2E)-3- (tetramethyl-1,3,2- dioxaborolan-2- yl)prop-2-enoate Yield: 29.3 mg; 0.05 mmol; 14%; white amorphous solid |
| 3K | 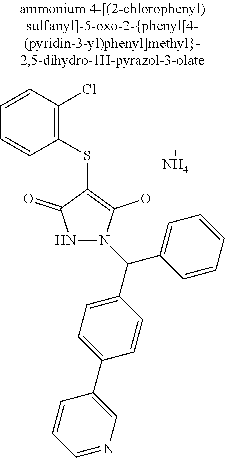 | ¹H NMR (400 MHz, D₂O) δ 8.80 (s, 1H), 8.47 (d, J = 4.6 Hz, 1H), 8.14 (d, J = 8.0 Hz, 1H), 7.71 (d, J = 7.9 Hz, 2H), 7.56- 7.44 (m, 3H), 7.45-7.36 (m, 5H), 7.25 (d, J = 8.1 Hz, 1H), 6.94 (t, J = 8.0 Hz, 1H), 6.85 (t, J = 7.6 Hz, 1H), 6.47 (s, 1H), 6.32 (d, J = 8.4 Hz, 1H). | M + 1 486.4 | Method 3: RT 5.6 min | Example 1H and (pyridin-3- yl)boronic acid Yield: 10 mg; 0.02 mmol; 17%; white amorphous solid. |
| 3L | 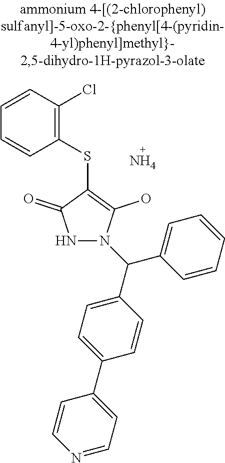 | ¹H NMR (400 MHz, D₂O) δ 8.80 (s, 1H), 8.47 (d, J = 4.6 Hz, 1H), 8.13 (s, 1H), 7.71 (d, J = 7.9 Hz, 2H), 7.49 (d, J = 8.0 Hz, 3H), 7.40 (s, 6H), 7.25 (d, J = 8.1 Hz, 1H), 6.93 (d, J = 8.2 Hz, 1H), 6.86 (d, J = 7.8 Hz, 1H), 6.47 (s, 1H), 6.32 (d, J = 7.7 Hz, 1H). | M + 1 486.4 | Method 3: RT 5.6 min | Example 1H and white amorphous solid, (pyridin-3- yl)boronic acid; Yield: 10 mg; 0.02 mmol; 17%; |
| 3M | 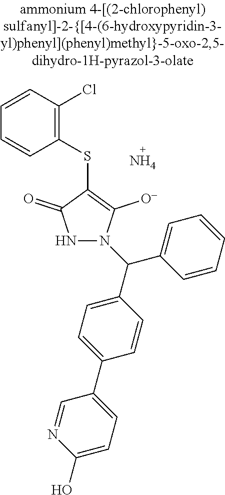 | ¹H NMR (400 MHz, MeOD) δ 8.01 (dd, J = 9.5, 2.7 Hz, 1H), 7.75 (dd, J = 2.6, 0.5 Hz, 1H), 7.59-7.53 (m, 2H), 7.46 (d, J = 8.2 Hz, 2H), 7.44-7.32 (m, 5H), 7.21-7.17 (m, 1H), 6.95- 6.86 (m, 2H), 6.68 (dd, J = 9.4, 0.5 Hz, 1H), 6.61-6.56 (m, 2H). | M + 1 502.7, M − 1 500.7 | Method 4: RT 6.4 min | Example 1H and 5-(tetramethyl- 1,3,2- dioxaborolan-2- yl)pyridin-2-ol Yield: 13 mg; 0.02 mmol; 21%; white amorphous solid |
| 3N | 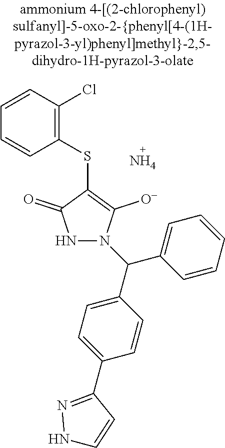 | ¹H NMR (400 MHz, MeOD) δ 7.84-7.77 (m, 2H), 7.68 (d, J = 2.2 Hz, 1H), 7.44 (s, 1H), 7.42-7.34 (m, 6H), 7.25-7.20 (m, 1H), 6.98- 6.91 (m, 2H), 6.72 (d, J = 2.2 Hz, 1H), 6.67 (s, 1H), 6.56 (dd, J = 6.1, 3.5 Hz, 1H). | M + 1 475.4, M − 1 473.05. | Method 4: RT 7.4 min, p 98.1% | Example 1H and 3-(tetramethyl- 1,3,2- dioxaborolan-2- yl)-1H-pyrazole Yield: 17 mg; 0.03 mmol; 28%; pale brown solid. |
| 3O | 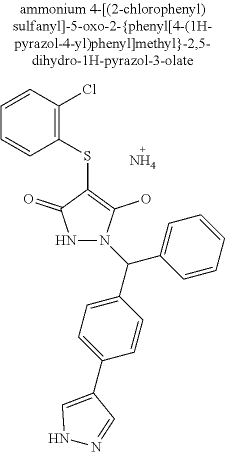 | ¹H NMR (400 MHz, MeOD) δ 8.01 (s, 2H), 7.62 (d, J = 8.3 Hz, 2H), 7.45- 7.33 (m, J = 8.3, 4.6 Hz, 7H), 7.25-7.18 (m, 1H), 6.98-6.89 (m, 2H), 6.63 (s, 1H), 6.59-6.53 (m, 1H) | M − 1 473.15. | Method 4: RT 7.0 min | Example 1H and 3-(tetramethyl- 1,3,2- dioxaborolan-2- yl)-1H-pyrazole Yield: 16 mg; 0.03 mmol; 26%; pale brown solid. |
| 3P | 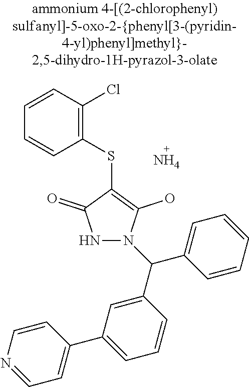 | 1H NMR (400 MHz, MeOD) δ 8.62 (dd, J = 5.2, 1.3 Hz, 2H), 7.94 (dd, J = 5.1, 1.5 Hz, 2H), 7.88-7.81 (m, 2H), 7.63-7.57 (m, 1H), 7.56- 7.50 (m, 1H), 7.47-7.30 (m, 5H), 7.17 (dd, J = 7.8, 1.2 Hz, 1H), 6.90 (td, J = 7.6, 1.5 Hz, 1H), 6.81 (td, J = 7.7, 1.3 Hz, 1H), 6.72 (s, 1H), 6.51 (dd, J = 7.9, 1.4 Hz, 1H). | M + 1 486.2, M − 1 484.15. | Method 4: RT 8.3 min | Example 1I and (pyridin-4- yl)boronic acid Yield: 18.6 mg; 0.03 mmol; 25%; white amorphous solid. |
| 3Q | 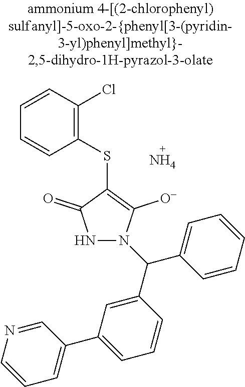 | ¹H NMR (400 MHz, MeOD) δ 8.83 (d, J = 1.8 Hz, 1H), 8.55 (dd, J = 5.0, 1.4 Hz, 1H), 8.21 (ddd, J = 8.0, 2.1, 1.6 Hz, 1H), 7.73-7.66 (m, 2H), 7.60 (ddd, J = 8.0, 5.1, 0.6 Hz, 1H), 7.55 (t, J = 7.8 Hz, 1H), 7.47-7.26 (m, 6H), 7.21 (dd, J = 7.8, 1.2 Hz, 1H), 6.93 (td, J = 7.6, 1.6 Hz, 1H), 6.85 (td, J = 7.7, 1.3 Hz, 1H), 6.74 (s, 1H), 6.53 (dd, J = 7.9, 1.5 Hz, 1H). | M + 1 486.15, M − 1 484.05 | Method 4: RT 8.4 min | Example 1I and (pyridin-3- yl)boronic acid Yield: 5.6 mg; 0.01 mmol; 7%; |
| 3R | 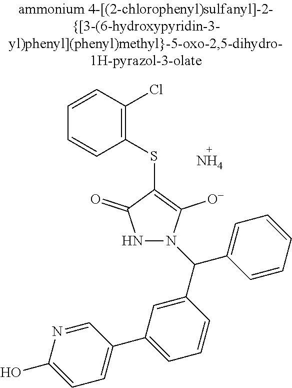 | ¹H NMR (400 MHz, MeOD) δ 7.93 (dd, J = 9.5, 2.7 Hz, 1H), 7.69-7.64 (m, 1H), 7.56-7.50 (m, 2H), 7.48- 7.42 (m, 1H), 7.42-7.28 (m, 6H), 7.20 (dd, J = 7.8, 1.3 Hz, 1H), 6.92 (td, J = 7.6, 1.6 Hz, 1H), 6.85 (td, J = 7.7, 1.3 Hz, 1H), 6.66 (s, 1H), 6.62 (dd, J = 9.5, 0.4 Hz, 1H), 6.52 (dd, J = 7.9, 1.5 Hz, 1H). | M + 1 502.25, M − 1 500.05 | Method 4: RT 6.7 min | Example 1I and 5- (tetramethyl-1,3,2- dioxaborolan-2- yl)pyridin-2-ol Yield: 15.2 mg; 0.03 mmol; 29%; white amorphous solid |
| 3S | 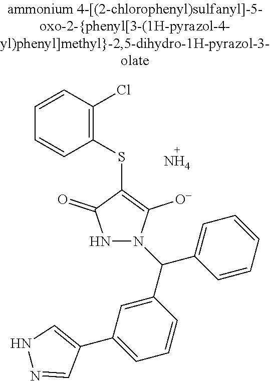 | ¹H NMR (400 MHz, MeOD) δ 8.83 (d, J = 1.8 Hz, 1H), 8.55 (dd, J = 5.0, 1.4 Hz, 1H), 8.21 (ddd, J = 8.0, 2.1, 1.6 Hz, 1H), 7.73-7.66 (m, 2H), 7.60 (ddd, J = 8.0, 5.1, 0.6 Hz, 1H), 7.55 (t, J = 7.8 Hz, 1H), 7.47-7.26 (m, 6H), 7.21 (dd, J = 7.8, 1.2 Hz, 1H), 6.93 (td, J = 7.6, 1.6 Hz, 1H), 6.85 (td, J = 7.7, 1.3 Hz, 1H), 6.74 (s, 1H), 6.53 (dd, J = 7.9, 1.5 Hz, 1H). | M + 1 475.3, M − 1 473.05. | Method 4: RT 7.3 min | Example 1I and 3- (tetramethyl-1,3,2- dioxaborolan-2- yl)-1H-pyrazole Yield: 14.8 mg; 0.03 mmol; 17%; pale brown solid. |
| 3T | 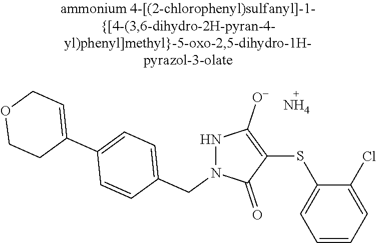 | not determined | M + 1 415.2 | Method 3: RT 9.5 min | Example 9V and 4-(4,4,5,5- Tetramethyl- [1,3,2]dioxa- borolan- 2-yl)-3,6- dihydro-2H-pyran Yield: 5.0 mg; 0.01 mmol; 10%; brown solid. |

### Example 4A

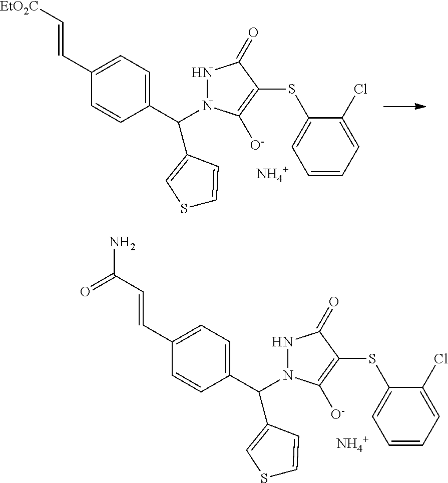 [SMILES: CCOC(=O)/C=C/c1ccc(C(c2ccsc2)n2[nH]c(=O)c(Sc3ccccc3Cl)c2[O-])cc1 ; [NH4+] ; NC(=O)/C=C/c1ccc(C(c2ccsc2)n2[nH]c(=O)c(Sc3ccccc3Cl)c2[O-])cc1 ; [NH4+]]

### Ammonium 2-({4-[(1E)-2-carbamoyleth-1-en-1-yl]phenyl}(thiophen-3-yl)methyl)-4-[(2-chlorophenyl)sulfanyl]-5-oxo-2,5-dihydro-1H-pyrazol-3-olate

Ammonium 4-[(2-chlorophenyl)sulfanyl]-2-({4-[(1E)-3-ethoxy-3-oxoprop-1-en-1-yl]phenyl}(thiophen-3-yl)methyl)-5-oxo-2,5-dihydro-1H-pyrazol-3-olate (Example 3J) (23.40 mg; 0.04 mmol; 1.00 eq.) is stirred at 50° C. for 3 h in the mixture of 5N NaOH aq. (50.00 μl; 0.25 mmol; 5.84 eq.) and methanol (1.00 ml). Then RM is neutralized with 1M HCl, evaporated to dryness and used in the next step without additional purification. The residue is dissolved in the mixture of ammonia solution 0.5 M in [1.4]-dioxane (1.00 ml; 0.50 mmol; 21.10 eq.) and DMF anhydrous (1.00 ml). RM is stirred overnight at rt, then diluted with AcOEt and washed with water. The water phase is acidified to pH 3 with 1 M HCl and extracted with AcOEt. All organic extracts are collected, dried over Na₂SO₄ and evaporated. The residue is dissolved in MeOH and subsequently 0.5 mL of 5N NaOH is added. Then RM is stirred overnight at rt, neutralized with 2M HCl and evaporated to dryness. The residue is purified by prep HPLC (C-18, Water/0.01% NH3-ACN/0.01% NH3) to obtain ammonium 2-({4-[(1E)-2-carbamoyleth-1-en-1-yl]phenyl}(thiophen-3-yl)methyl)-4-[(2-chlorophenyl)sulfanyl]-5-oxo-2,5-dihydro-1H-pyrazol-3-olate (3.90 mg; 0.01 mmol; 31.1%; white amorphous solid). Method 3: RT 9.4 min, p 94.7%, M−1 482.05. ¹H NMR (400 MHz, D20) δ 7.59 (d, J=8.3 Hz, 2H), 7.51 (d, J=16.0 Hz, 1H), 7.46-7.39 (m, 3H), 7.30-7.25 (m, 1H), 7.23 (dd, J=7.9, 1.3 Hz, 1H), 7.11 (dd, J=5.0, 1.3 Hz, 1H), 6.95 (td, J=7.6, 1.6 Hz, 1H), 6.90-6.83 (m, 1H), 6.63 (d, J=15.9 Hz, 1H), 6.37 (s, 1H), 6.15 (dd, J=7.9, 1.5 Hz, 1H).

### Example 5A

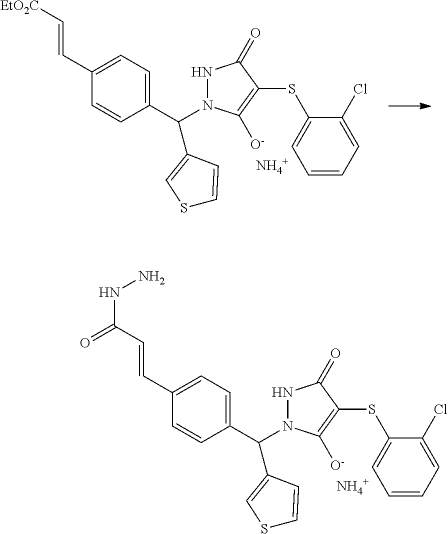 [SMILES: CCOC(=O)/C=C/c1ccc(C(c2ccsc2)n2[nH]c(=O)c(Sc3ccccc3Cl)c2[O-])cc1 ; [NH4+] ; NNC(=O)/C=C/c1ccc(C(c2ccsc2)n2[nH]c(=O)c(Sc3ccccc3Cl)c2[O-])cc1 ; [NH4+]]

### Ammonium 4-[(2-chloropheny)sulfanyl)sulfanyl]-2-({4-[(1E)-2-(hydrazinecarbonyl)eth-1-en-1-yl]phenyl}(thiophen-3-yl)methyl)-5-oxo-2,5-dihydro-1H-pyrazol-3-olate

Ammonium 4-[(2-chlorophenyl)sulfanyl]-2-({4-[(1E)-3-ethoxy-3-oxoprop-1-en-1-yl]phenyl}(thiophen-3-yl)methyl)-5-oxo-2,5-dihydro-1H-pyrazol-3-olate (Example 3J) (90.00 mg; 0.17 mmol; 1.00 eq.) is dissolved in anhydrous ethanol (3.00 ml) then hydrazine monohydrate (0.04 ml; 0.66 mmol; 4.00 eq.) is added and RM is stirred at 1200° C. overnight. RM is evaporated and the residue is purified by prep HPLC (C-18, Water/0.01% NH3-ACN/0.01% NH3) to obtain ammonium 4-[(2-chlorophenyl)sulfanyl]-2-({4-[(1E)-2-(hydrazinecarbonyl)eth-1-en-1-yl]phenyl}(thiophen-3-yl)methyl)-5-oxo-2,5-dihydro-1H-pyrazol-3-olate (11.00 mg; 0.02 mmol; 12%; beige solid). Method 4: RT 5.6 min, p 90.2%, M+1 501.3, M−1 499.9. 1H NMR (400 MHz, MeOD) 7.45 (dd, J=5.0, 3.0 Hz, 1H), 7.35-7.29 (m, 3H), 7.27-7.21 (m, 3H), 7.10 (dd, J=5.0, 1.2 Hz, 1H), 7.03-6.94 (m, 2H), 6.59 (dd, J=6.4, 3.1 Hz, 2H), 2.97 (t, J=7.6 Hz, 2H), 2.50 (t, J=7.6 Hz, 2H).

### Example 6A

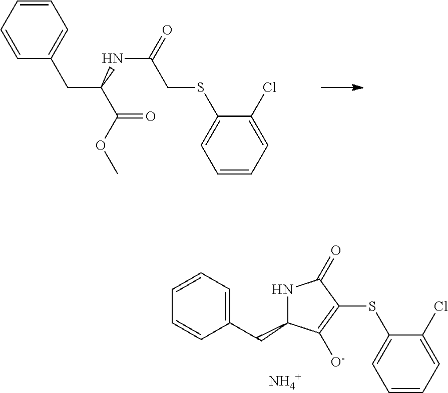 [SMILES: COC(=O)[C@@H](Cc1ccccc1)NC(=O)CSc1ccccc1Cl ; O=C1N[C@H](Cc2ccccc2)C([O-])=C1Sc1ccccc1Cl ; [NH4+]]

### Ammonium (S)-2-benzyl-4-[(2-chlorophenyl)sulfanyl]-5-oxo-2,5-dihydro-1H-pyrrol-3-olate

The solution of methyl (2S)-2-{2-[(2-chlorophenyl)sulfanyl]acetamido}-3-phenylpropanoate (Intermediate 12A)(90.00 mg; 0.24 mmol; 1.00 eq.) in toluene (3.00 ml) is added dropwise to a refluxed mixture of sodium t-butoxide (70.00 mg; 0.73 mmol; 2.99 eq.) in toluene (4.00 ml). The heating is continued for 2.5 h. Then the solvent is removed under reduced pressure and the residue is purified using prep. HPLC (C-18, Water/0.01% NH3-ACN/0.01% NH3) to obtain ammonium (S)-2-benzyl-4-[(2-chlorophenyl)sulfanyl]-5-oxo-2,5-dihydro-1H-pyrrol-3-olate (40.00 mg; 0.11 mmol; 47.1%; white fine powder, ee—16%). Method 3: RT 8.3 min, p 100%, M+1 332.0, M−1 330.0). ¹H NMR (400 MHz, D20/DMSO) δ 9.52-9.33 (m, 6H), 9.12 (td, J=7.7, 1.5 Hz, 1H), 8.99 (td, J=7.8, 1.3 Hz, 1H), 7.83 (dd, J=7.9, 1.4 Hz, 1H), 6.40 (t, J=4.2 Hz, 1H), 5.29-5.13 (m, 2H)

The following compounds are prepared by the procedure for Example 6A, using the appropriate starting materials:

| | | | | | Starting |
|---|---|---|---|---|---|
| Ex. | | | | | materials and |
| No. | Name and structure | NMR | mz | RT | yield |
| 6B | 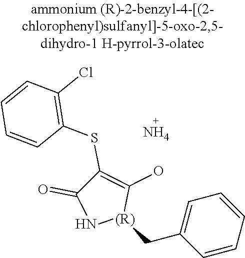 | ¹H NMR (400 MHz, DMSO) δ 7.33-7.17 (m, 6H), 6.99-6.92 (m, 1H), 6.89-6.81 (m, 1H), 5.85 (dd, J = 7.8, 1.0 Hz, 1H), 4.17 (t, J = 4.5 Hz, 1H), 3.05-2.94 (m, 2H). | M + 1 332.0, M − 1 330.0 | Method 3: RT 8.3 min | Intermediate 12B; Yield: 17.6 mg; 0.05 mmol; 21%; light beige powder. |
| 6C | 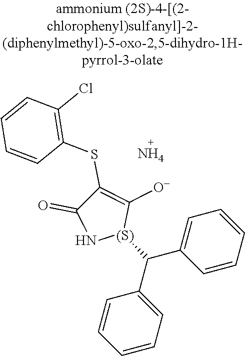 | ¹H NMR (400 MHz, MeOD) δ 7.41-7.19 (m, 10H), 7.13 (dd, J = 7.8, 1.4 Hz, 1H), 6.86 (td, J = 7.6, 1.6 Hz, 1H), 6.79 (td, J = 7.6, 1.4 Hz, 1H), 5.87 (dd, J = 7.9, 1.5 Hz, 1H), 4.76 (d, J = 2.6 Hz, 1H), 4.70 (d, J = 2.7 Hz, 1H). | M − 1 406.5 | Method 3: RT 13.20 min, p 97.6%, | Intermediate 12C; Yield: 85 mg; 0.20 mmol; 34%. |
| 6D | 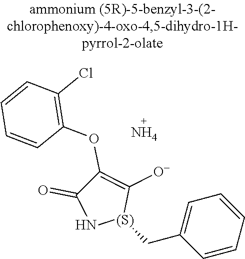 | ¹H NMR (400 MHz, MeOD) δ: 8.43 (brs, 1H), 7.30 (s, 5H), 7.27-7.24 (m, 1H), 6.89-6.77 (m, 2H), 5.74-5.64 (m, 1H), 4.25 (t, J = 4.4 Hz, 1H), 3.15 (dd, J = 13.8, 4.0 Hz, 1H), 3.08 (dd, J = 13.8, 4.8 Hz, 1H). | M + 1 316.2 | Method 2: RT 5.9 min, p 96.8%. | Intermediate 12D; Yield: 28 mg; 0.11 mmol; 38%. |
| 6E | 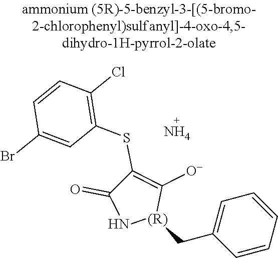 | 1H NMR (400 MHz, MeOD): 7.33-7.17 (m, 7H), 6.74 (dd, J = 1.6, 0.6 Hz, 1H), 4.53 (dd, J = 5.8, 4.5 Hz, 1H), 3.27-3.19 (m, 1H), 3.01 (dd, J = 13.9, 6.0 Hz, 1H) | M + H+; 412.5 | Method 2: RT: 7.0 min., 95.9%, | Intermediate 12E; Yield: 6.8% White powder |
| 6F | 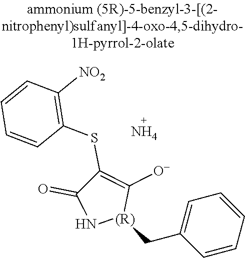 | ¹H NMR (400 MHz, MeOD) δ 8.18-8.13 (m, 1H), 8.11-8.05 (m, 1H), 7.77-7.69 (m, 2H), 7.34-7.29 (m, 2H), 7.26-7.19 (m, 2H), 7.18-7.12 (m, 1H), 4.76 (dd, J = 7.6, 4.8 Hz, 1H), 3.36 (dd, J = 13.8, 4.8 Hz, 1H), 3.17 (dd, J = 13.8, 7.6 Hz, 1H). | M + 1 343.2 | Method 2: RT 6.0 min, p 88.8%. | Intermediate 12F; Yield: 9.6 mg; 0.04 mmol; 9.6%. |
| 6G | 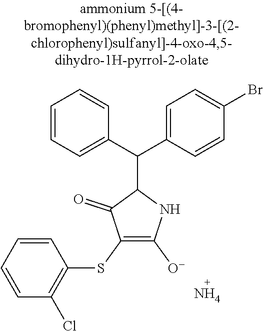 | 1H NMR (400 MHz, MeOD) δ 7.51-7.42 (m, 4H), 7.39-7.23 (m, 10H), 7.22-7.15 (m, 2H), 6.98-6.88 (m, 4H), 6.85-6.79 (m, 2H), 5.84-5.80 (m, 2H), 5.78 (dd, J = 7.9, 1.5 Hz, 2H), 4.86 (d, J = 2.7 Hz, 2H), 4.75 (d, J = 2.6 Hz, 1H), 4.71 (d, J = 2.8 Hz, 1H). Enantiomers in 1:1 molar ratio based on signals of methine group at 4.71 ppm and 4.75 ppm | M + 1 488.2, M − 1 485.9 | Method 3: RT 14.4 min, p 96.6% | Intermediate 12G Yield: 562 mg; 1.11 mmol; 48%. |

### Example 7A

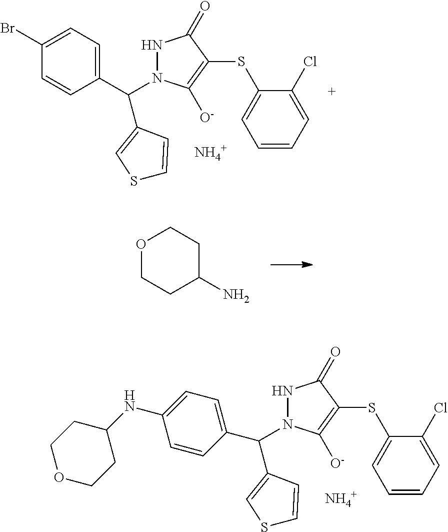 [SMILES: O=c1[nH]n(C(c2ccc(Br)cc2)c2ccsc2)c([O-])c1Sc1ccccc1Cl ; [NH4+] ; NC1CCOCC1 ; O=c1[nH]n(C(c2ccc(NC3CCOCC3)cc2)c2ccsc2)c([O-])c1Sc1ccccc1Cl ; [NH4+]]

### Ammonium 4-[(2-chlorophenyl)sulfanyl]-1-{[4-(1,4-oxazepan-4-yl)phenyl](phenyl)methyl}-5-oxo-2,5-dihydro-1H-pyrazol-3-olate

Ammonium 5-[(4-bromophenyl)(thiophen-3-yl)methyl]-3-[(2-chlorophenyl)sulfanyl]-4-oxo-4,5-dihydro-1H-pyrrol-2-olate (Example 1C) (1 mmol), Brettphos (0.10 eq.), BrettPhos Pd G1 Methyl-t-Butyl Ether Adduct (0.05 eq.) are placed in a glass reactor vial. The vial is capped and air is evacuated, backfilled with argon (these steps are repeated 3 times). Then the solution of sodium t-butoxide 2M in THF (7.00 eq.), primary amine 4-aminotetrahydropyrane (2.00 eq.) are added and the reaction mixture is heated in an oil bath for 18 h at 65° C. Then the RM is diluted with methanol and passed through a pad of celite. The filtrate is evaporated and the residue is diluted with AcOEt/hexane (1:1) mixture and extracted with 2% aq ammonia. Subsequently the aqueous extract is washed with mixture of EtOAc/Hexane and evaporated again. The residue is purified by prep HPLC (C-18, Water/0.01% NH3-ACN/0.01% NH3). Yield: 8 mg; 0.01 mmol, 10% white amorphous solid. Method 4: RT 7.8 min, p 94%, 508.2 (M+1). 1H NMR (400 MHz, D20) δ 7.40-7.30 (m, 6H), 7.27-7.23 (m, 1H), 7.17 (d, J=8.5 Hz, 2H), 7.00-6.94 (m, 2H), 6.82 (d, J=8.6 Hz, 2H), 6.40-6.34 (m, 1H), 6.30 (s, 1H), 3.94 (d, J=11.2 Hz, 2H), 3.55-3.43 (m, 3H), 1.98-1.87 (m, 2H), 1.49-1.33 (m, 2H).

The following compounds are prepared by the procedure for Example 7A, using the appropriate starting materials:

| | | | | | Starting |
|---|---|---|---|---|---|
| Ex. | | | | | materials and |
| No. | Name and structure | NMR | mz | RT | yield |
| 7B | 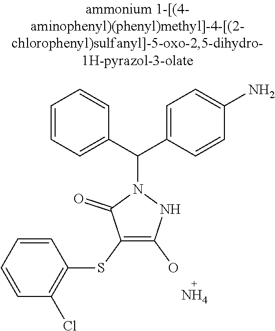 | 1H NMR (400 MHz, D2O) δ 7.40-7.28 (m, 5H), 7.28-7.21 (m, 1H), 7.13 (d, J = 8.3 Hz, 2H), 7.01- 6.94 (m, 2H), 6.81- 6.75 (m, 2H), 6.35 (dd, J = 5.6, 3.9 Hz, 1H), 6.29 (s, 1H). | 422.4 (M − 1), 424.4 (M + 1). | Method 4: RT 6.2 min | Example 1H and Ammonia; Yield: 16 mg; 0.04 mmol, 25% white amorphous solid. |
| 7C | 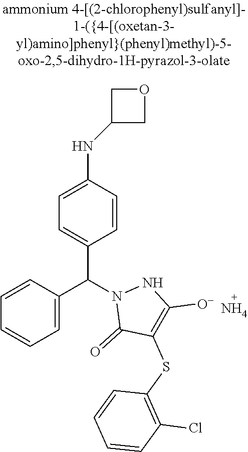 | 1H NMR (400 MHz, D2O) δ 7.40-7.29 (m, 5H), 7.25 (dd, J = 7.6, 1.6 Hz, 1H), 7.20- 7.14 (m, 2H), 7.02- 6.92 (m, 2H), 6.67- 6.62 (m, 2H), 6.33 (dd, J = 7.6, 1.8 Hz, 1H), 6.29 (s, 1H), 4.98 (t, J = 6.7 Hz, 2H), 4.63- 4.59 (m, 1H), 4.53 (t, J = 5.8 Hz, 2H). | 477.9 (M − 1) | Method 4: RT 7.0 min | Example 1H and oxetan-3- amine; Yield: 6.4 mg; 0.01 mmol, 9% white amorphous solid |
| 7D | 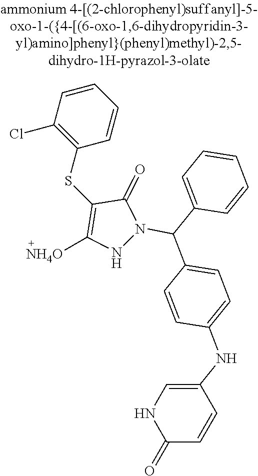 | 1H NMR (400 MHz, MeOD) δ 7.60 (dd, J = 9.6, 3.0 Hz, 1H), 7.39- 7.32 (m, 5H), 7.28 (dd, J = 2.9, 0.6 Hz, 1H), 7.23-7.18 (m, 3H), 7.01-6.92 (m, 2H), 6.85-6.80 (m, 2H), 6.59 (ddd, J = 6.5, 4.9, 1.2 Hz, 2H), 6.51 (s, 1H). | 515.6 (M − 1) | Method 4: RT 8.5 min | Example 1H and 5-amino- 2-pyridone; Yield: 50 mg; 0.09 mmol, 63% light brown amorphous solid |
| 7E | 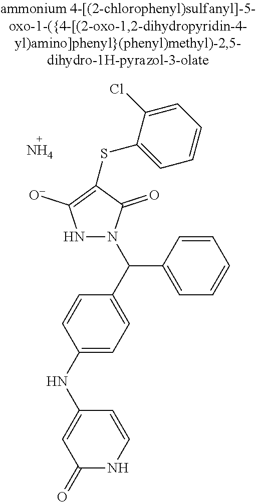 | 1H NMR (400 MHz, MeOD) δ 7.42-7.33 (m, 7H), 7.28-7.18 (m, 4H), 7.00-6.92 (m, 2H), 6.63-6.57 (m, 2H), 6.22 (dd, J = 7.3, 2.3 Hz, 1H), 6.00 (d, J = 2.2 Hz, 1H). | 515.3 (M − 1). | Method 4: RT 6.0 min | Example 1H and 4-amino- 2-pyridone; Yield: 18 mg; 0.03 mmol, 23% white powder. |
| 7F | 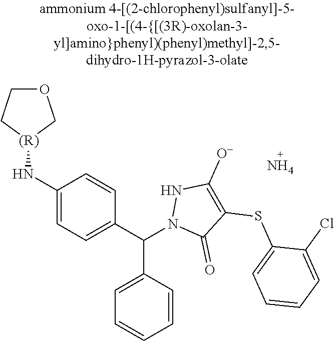 | 1H NMR (400 MHz, MeOD) δ 7.43-7.28 (m, 6H), 7.17 (dt, J = 13.3, 2.0 Hz, 3H), 6.99- 6.89 (m, 2H), 6.68- 6.63 (m, 2H), 6.61- 6.55 (m, 1H), 6.45 (s, 1H), 4.14-4.07 (m, 1H), 4.02-3.92 (m, 2H), 3.89-3.80 (m, 2H), 3.69 (dd, J = 8.7, 3.4 Hz, 1H), 2.31- 2.23 (m, 1H), 1.95- 1.89 (m, 1H). | %, 493.8 (M − 1). | Method 4: RT 7.5 min | Example 1H and 3-(R)- tetrahydrofuran; Yield: 11 mg; 0.02 mmol, 11% beige solid |
| 7G | 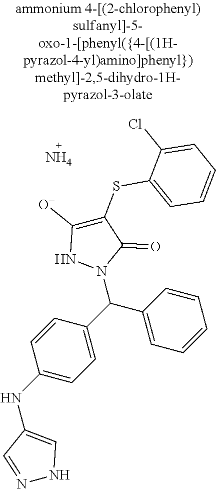 | 1H NMR (400 MHz, MeOD) δ 7.54 (s, 2H), 7.42-7.29 (m, 9H), 7.21-7.15 (m, 2H), 6.92 (dtd, J = 18.5, 7.4, 1.6 Hz, 2H), 6.84- 6.79 (m, J = 8.7 Hz, 2H), 6.55 (dd, J = 7.7, 1.8 Hz, 1H), 6.44 (s, 1H). | 489.5 (M − 1). | Method 4: RT 6.6 min | Example 1H and 1H- pyrazol-4- amine; Yield: 6 mg; 0.01 mmol, 7% beige solid. |
| 7H | 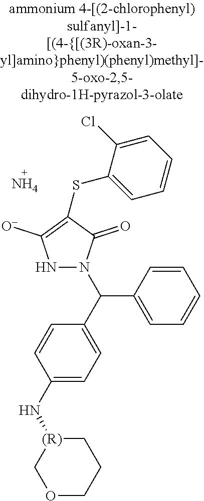 | 1H NMR (400 MHz, MeOD) δ 7.42-7.28 (m, 5H), 7.21-7.11 (m, 3H), 6.99-6.88 (m, 2H), 6.71-6.64 (m, 2H), 6.62-6.57 (m, 1H), 6.43 (s, 1H), 4.00 (dd, J = 11.0, 2.5 Hz, 1H), 3.86-3.78 (m, 1H), 3.54-3.42 (m, 2H), 3.26 (ddd, J = 11.0, 8.3, 2.8 Hz, 1H), 2.14-2.01 (m, 1H), 1.86-1.75 (m, 1H), 1.75-1.62 (m, 1H), 1.62-1.51 (m, 1H). | 507.8 (M + 1). | Method 4: RT 8.4 min | Example 1H and (3R)- aminotetra- hydropyran; Yield: 7 mg; 0.01 mmol, 9% white amorphous solid |
| 7I | 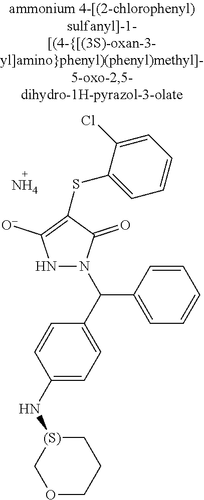 | 1H NMR (400 MHz, MeOD) δ 7.42-7.26 (m, 5H), 7.18 (dd, J = 7.6, 1.5 Hz, 1H), 7.14 (d, J = 8.5 Hz, 2H), 6.99-6.88 (m, 2H), 6.70-6.64 (m, 2H), 6.61-6.56 (m, 1H), 6.42 (s, 1H), 4.11- 3.95 (m, 2H), 3.82 (dt, J = 10.6, 3.9 Hz, 1H), 3.54-3.43 (m, 2H), 3.26 (ddd, J = 10.9, 8.1, 2.7 Hz, 1H), 2.08 (d, J = 8.5 Hz, 1H), 1.84-1.51 (m, 3H). | 506.32 (M − 1). | Method 4: RT 8.3 min | Example 1H and (3S)- aminotetra- hydropyran; Yield: 36 mg; 0.06 mmol, 46% white amorphous solid |
| 7J | 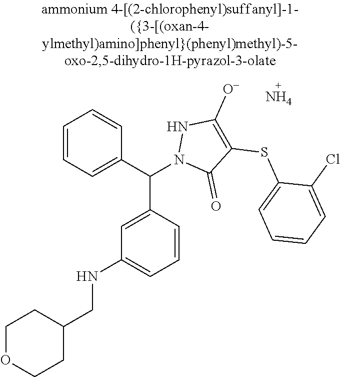 | 1H NMR (400 MHz, MeOD) δ 7.42-7.31 (m, 6H), 7.21-7.18 (m, 1H), 7.11 (t, J = 7.8 Hz, 1H), 6.97- 6.92 (m, 2H), 6.63 (ddd, J = 6.4, 4.0, 1.5 Hz, 3H), 6.48 (s, 1H), 3.96-3.88 (m, 2H), 3.46-3.34 (m, 3H), 2.96 (d, J = 6.8 Hz, 2H), 1.91-1.77 (m, 1H), 1.73-1.64 (m, 2H). | 521.6 (M − 1) 522.2 (M + 1). | Method 3: RT 10.3 min | Example 1I and (oxan-4- yl)methanamine; Yield: 8 mg; 0.01 mmol, 10% white solid |
| 7K | 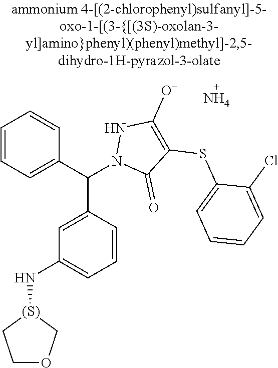 | | 493.0 (M − 1), 494.1 (M + 1). | Method 3: RT 11.2 min, P 93% | Example 1I and oxolan- (3S)-amine; Yield: 3 mg; 0.01 mmol, 4% white solid |
| 7L | 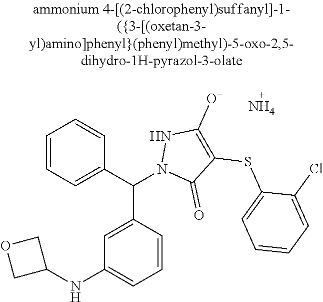 | 1H NMR (400 MHz, MeOD) δ 7.43-7.31 (m, 5H), 7.21-7.16 (m, 1H), 7.13 (t, J = 7.8 Hz, 1H), 6.99- 6.89 (m, 2H), 6.69 (d, J = 7.7 Hz, 1H), 6.66- 6.61 (m, 1H), 6.56 (s, 1H), 6.51 (dd, J = 8.0, 1.7 Hz, 1H), 6.46 (s, 1H), 4.92 (td, J = 6.2, 4.5 Hz, 2H), 4.59 (dt, J = 12.2, 6.1 Hz, 1H), 4.52 (td, J = 5.9, 2.6 Hz, 2H) | 480.2 (M + 1) 478.1 (M − 1) | Method 4: RT 7.2 min, P 97%, | Example 1I and oxetan-3- amine; Yield: 9 mg; 0.02 mmol, 13% white solid |
| 7M | 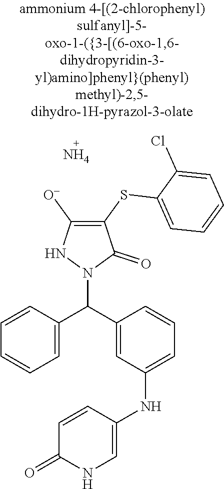 | 1H NMR (400 MHz, D2O) δ 7.54-7.50 (m, 5H), 7.39-7.36 (m, 7H), 7.34-7.31 (m, 5H), 7.28 (d, J = 1.8 Hz, 3H), 7.27-7.21 (m, 4H), 7.02-6.95 (m, 5H), 6.37 (s, 2H), 6.30-6.26 (m, 2H). | 515.6 (M − 1), 517.0 (M + 1). | Method 4: RT 6.5 min, P 98% | Example 1I and 5- aminopyridin- 2-ol; Yield: 38 mg; 0.06 mmol, 47% white solid |
| 7N | 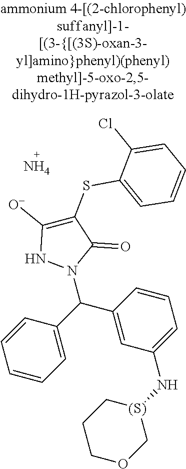 | 1H NMR (400 MHz, MeOD) δ 7.45-7.33 (m, 5H), 7.32-7.25 (m, 1H), 7.20 (t, J = 7.8 Hz, 1H), 7.09- 7.00 (m, 2H), 6.75 (dd, J = 7.9, 2.2 Hz, 1H), 6.71 (s, 1H), 6.67 (d, J = 7.1 Hz, 1H), 6.64- 6.58 (m, 2H), 3.93 (ddd, J = 11.2, 3.4, 1.5 Hz, 1H), 3.78 (dt, J = 11.2, 4.3 Hz, 1H), 3.46 (dddd, J = 20.6, 12.5, 8.7, 3.6 Hz, 2H), 3.30- 3.23 (m, 1H), 1.86- 1.49 (m, 4H). | 508.1 (M + 1) 506.7 (M − 1). | Method 3: RT 8.6 min, P 93% | Example 1I and (3S)- oxan-3- amine; Yield: 23 mg; 0.05 mmol, 33% light brown solid |
| 7O | 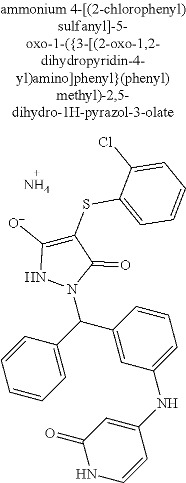 | 1H NMR (400 MHz, MeOD) □ 7.44-7.30 (m, 7H), 7.26-7.23 (m, 1H), 7.23-7.15 (m, 3H), 6.94-6.84 (m, 2H), 6.59 (s, 1H), 6.46-6.38 (m, 1H), 6.28 (dd, J = 7.3, 2.2 Hz, 1H), 6.14 (d, J = 2.2 Hz, 1H). | 514.9 (M − 1), 517.25 (M + 1). | Method 4: RT 6.2 min, P 96% | Example 1I and 4- aminopyridin- 2-ol; Yield: 13 mg; 0.02 mmol, 17% white yellow solid. |
| 7P | 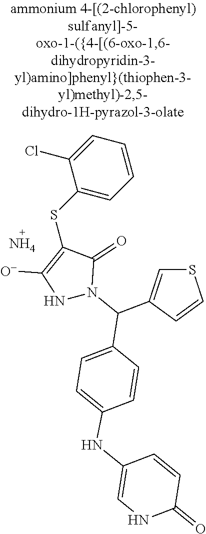 | 1H NMR (400 MHz, D2O) δ 7.55 (dd, J = 9.6, 2.9 Hz, 1H), 7.41 (dd, J = 5.0, 3.0 Hz, 1H), 7.29 (d, J = 2.8 Hz, 1H), 7.23 (d, J = 8.1 Hz, 4H), 7.10- 7.07 (m, 1H), 6.92 (qd, J = 7.4, 5.7 Hz, 2H), 6.81 (d, J = 8.6 Hz, 2H), 6.56 (d, J = 9.6 Hz, 1H), 6.26 (s, 1H), 6.20 (dd, J = 7.5, 1.9 Hz, 1H). | 521.8 (M − 1). | Method 4: RT 5.8 min, P 90% | Example 1C and 5- aminopyridin- 2-ol; Yield: 43 mg; 0.07 mmol, 52% light brown-purple solid. |
| 7Q | 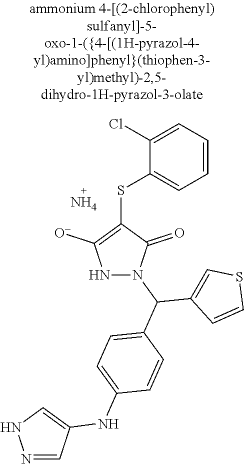 | 1H NMR (400 MHz, D2O) δ 7.56 (d, J = 2.3 Hz, 1H), 7.43 (dd, J = 5.0, 3.0 Hz, 1H), 7.28 (d, J = 8.8 Hz, 3H), 7.25-7.20 (m, 1H), 7.14-7.08 (m, 3H), 6.95-6.88 (m, 2H), 6.28 (s, 1H), 6.17- 6.12 (m, 1H), 6.03 (d, J = 2.4 Hz, 1H). | 414.0 (M − 1). | Method 4: RT 6.9 min, P 99% | Example 1C and 1H- pyrazol-4- amine; Yield: 15 mg; 0.03 mmol, 20% light créme powder |
| 7R | 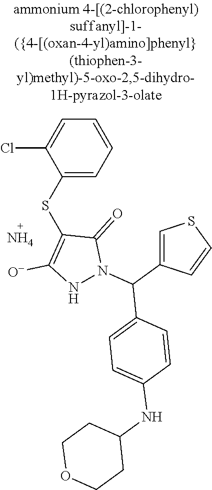 | 1H NMR (400 MHz, D2O) δ 7.41 (dd, J = 5.0, 3.0 Hz, 1H), 7.26- 7.19 (m, 4H), 7.08 (dd, J = 5.0, 1.3 Hz, 1H), 6.99-6.94 (m, 2H), 6.79 (d, J = 8.6 Hz, 2H), 6.31-6.23 (m, 2H), 3.98-3.87 (m, 2H), 3.56-3.41 (m, 3H), 1.96-1.88 (m, 2H), 1.46-1.30 (m, 2H) | 513.5 (M − 1). | Method 4: RT 7.5 min, P 97% | Example 1C and oxan-4- amine; Yield: 33 mg; 0.06 mmol, 36% white amorphous solid |
| 7S | 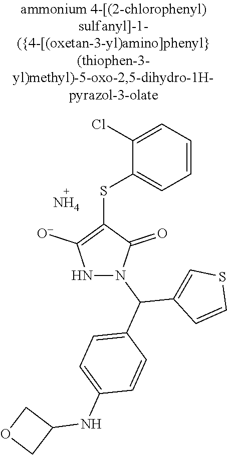 | 1H NMR (400 MHz, D2O) δ 7.44 (dd, J = 4.9, 3.0 Hz, 1H), 7.25 (d, J = 8.7 Hz, 4H), 7.10 (d, J = 5.1 Hz, 1H), 7.05-6.91 (m, 2H), 6.66 (d, J = 8.5 Hz, 2H), 6.29 (s, 1H), 6.27-6.22 (m, 1H), 5.00 (t, J = 6.7 Hz, 2H), 4.66-4.58 (m, 1H), 4.55 (t, J = 6.2 Hz, 2H). | 488.1 (M + 1). | Method 4: RT 6.8 min, P 95% | Example 1C and oxetan-3- amine; Yield: 4 mg; 0.01 mmol, 6% white amorphous solid. |
| 7T | 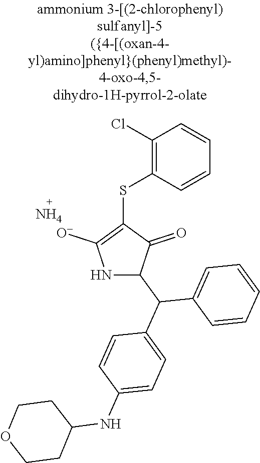 | 1H NMR (400 MHz, MeOD) δ 7.37 (dd, J = 7.5, 1.8 Hz, 2H), 7.31- 7.21 (m, 3H), 7.15 (d, J = 8.4 Hz, 2H), 7.11 (dd, J = 7.7, 1.4 Hz, 1H), 6.84 (td, J = 7.6, 1.7 Hz, 1H), 6.81- 6.75 (m, 1 H), 6.69 (d, J = 8.6 Hz, 2H), 5.91 (d, J = 7.5 Hz, 1H), 4.65 (d, J = 2.4 Hz, 1H), 4.52 (d, J = 2.6 Hz, 1H), 4.03- 3.92 (m, 2H), 3.61- 3.45 (m, 3H), 2.04- 1.97 (m, 2H), 1.55- 1.41 (m, 2H). | M + 1 507.3. | Method 3: RT 5.6 min, P 93.8% | Example 6G and oxan-4- amine; Yield: 10.9 mg; 0.02 mmol, 14% beige solid. |

### Example 8A

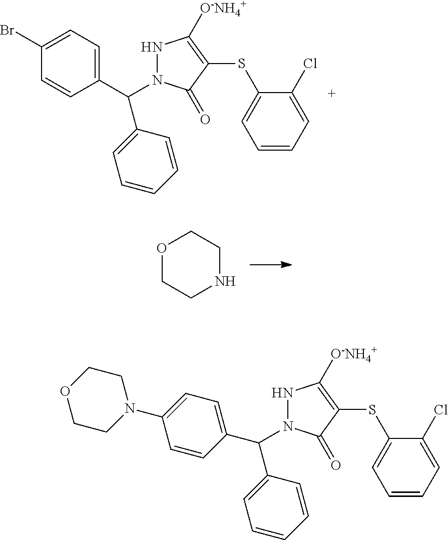

### Ammonium 4-[(2-chlorophenyl)sulfanyl]-2-{[4-(morpholin-4-yl)phenyl](phenyl)methyl}-5-oxo-2,5-dihydro-1H-pyrazol-3-olate

Ammonium 5-[(4-bromophenyl)phenyl)methyl]-3-[(2-chlorophenyl)sulfanyl]-4-oxo-4,5-dihydro-1H-pyrrol-2-olate (Example 1H) (1 mmol), RuPhos Pd G2 (0.06 eq.), and RuPhos (0.06 eq.) are placed in a glass reactor vial. The vial is capped and air is evacuated, backfilled with argon (these steps are repeated 3 times). Then solution of sodium t-butoxide 2M in THF (7.00 eq.) and primary amine—morpholine (2.00 eq.) are added and the reaction mixture is heated in an oil bath for 18 h at 65° C. Then the RM is diluted with methanol and passed through a pad of celite. The filtrate is evaporated and the residue is diluted with AcOEt and hexane (1:1) and extracted with 2% aq ammonia.

Subsequently the aqueous extract is washed with mixture of EtOAc/Hexane and evaporated again. The residue is purified by prep HPLC (C-18, Water/0.01% NH3-ACN/0.01% NH3). Yield: 42 mg; 0.08 mmol, 20% white amorphous solid. Method 3: RT 10.2 min, p 100%, 493.4 (M+1). 1H NMR (400 MHz, D20) δ 7.52-7.31 (m, 7H), 7.20-7.14 (m, 1H), 7.04 (d, J=8.2 Hz, 2H), 6.90-6.81 (m, 1H), 6.71-6.62 (m, 1H), 6.01 (s, 1H), 3.89-3.76 (m, 4H), 3.14 (s, 4H).

The following compounds are prepared by the procedure for Example 8A, using the appropriate starting materials:

| | | | | | Starting |
|---|---|---|---|---|---|
| Ex. | | | | | materials |
| No. | Name and structure | NMR | mz | RT | and Yield |
| 8B | 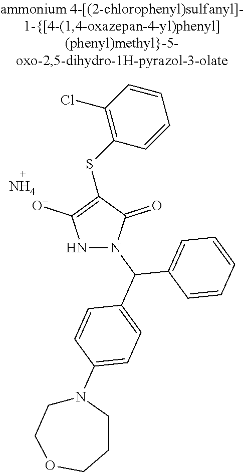 | 1H NMR (400 MHz, D2O) δ 7.41-7.30 (m, 5H), 7.28-7.22 (m, 1H), 7.22- 7.16 (m, 2H), 7.00-6.94 (m, 2H), 6.86-6.80 (m, 2H), 6.41- 6.36 (m, 1H), 6.30 (s, 1H), 3.90-3.74 (m, 2H), 3.68-3.54 (m, 4H), 3.34- 3.25 (m, 2H), 1.97-1.91 (m, 2H). | 506.6 (M + 1). | Method 4: RT 8.5 min, P 100% | Example 1H and homo- morpholine; Yield: 3 mg; 0.01 mmol, 5% white amorphous solid. |
| 8C | 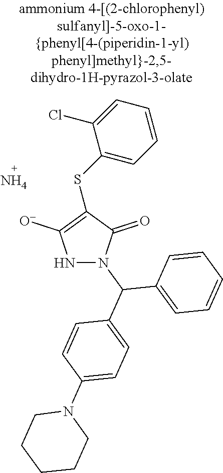 | 1H NMR (400 MHz, MeOD) δ 7.41-7.29 (m, 6H), 7.28-7.23 (m, 2H), 7.20- 7.17 (m, 1H), 7.01-6.97 (m, 2H), 6.93 (ddd, J = 14.2, 7.2, 1.8 Hz, 2H), 6.61- 6.58 (m, 1H), 6.48 (s, 1H), 3.20-3.16 (m, 2H), 2.98-2.94 (m, 2H), 1.78- 1.71 (m, 2H), 1.71-1.65 (m, 4H). | 490.4 (M − 1) 492.1 (M + 1). | Method 4: RT 10.6 min, P 97% | Exampe 1H and piperidine; Yield: 8 mg; 0.01 mmol, 10% beige powder |
| 8D | 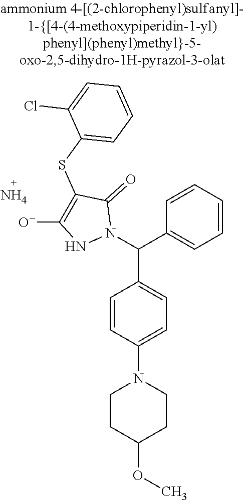 | 1H NMR (400 MHz, MeOD) δ 7.40-7.31 (m, 5H), 7.27 (d, J = 8.5 Hz, 2H), 7.23-7.20 (m, 1H), 7.07 (d, J = 8.8 Hz, 2H), 7.01-6.93 (m, 2H), 6.62-6.58 (m, 1H), 6.54 (s, 1H), 3.63-3.55 (m, 2H), 3.48- 3.43 (m, 1H), 3.41 (s, 3H), 3.04 (ddd, J = 9.4, 6.2, 2.3 Hz, 2H), 2.08 (d, J = 15.3 Hz, 2H), 1.78-1.68 (m, 2H) | 522.1 (M + 1) 520.7 (M − 1). | Method 4: RT 6.5 min, P 96% | Example 1H and 4- methoxy- piperidine; Yield: 5 mg; 0.01 mmol, 6% beige solid |
| 8E | 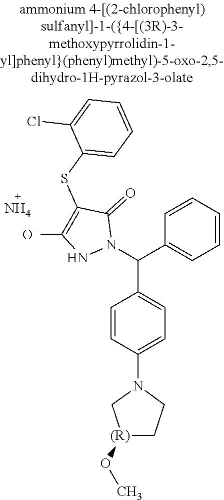 | 1H NMR (400 MHz, MeOD) δ 7.42-7.27 (m, 6H), 7.24-7.20 (m, 2H), 7.19- 7.15 (m, 1H), 6.92-6.87 (m, 2H), 6.61-6.56 (m, 2H), 6.55- 6.51 (m, 1H), 6.45 (s, 1H), 4.19-4.14 (m, 1H), 3.54-3.48 (m, 1H), 3.41- 3.39 (m, 3H), 3.39-3.36 (m, 2H), 2.20-2.14 (m, 2H). | 508.3 (M + 1), 508.6 (M − 1). | Method 4: RT 9.4 min, P 92% | Example 1H and (3R)-3- methoxy- pyrrolidine; Yield: 12 mg; 0.02 mmol, 15% beige powder. |
| 8F | 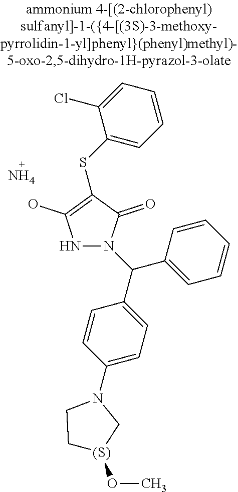 | 1H NMR (400 MHz, MeOD) δ 7.34 (ddt, J = 22.4, 9.7, 7.0 Hz, 5H), 7.22 (d, J = 8.6 Hz, 2H), 7.19-7.14 (m, 1H), 6.93-6.86 (m, 2H), 6.59 (d, J = 8.7 Hz, 2H), 6.56-6.50 (m, 1H), 6.45 (s, 1H), 4.21-4.12 (m, 1H), 3.54- 3.47 (m, 1H), 3.39 (d, J = 2.3 Hz, 3H), 3.20- 2.99 (m, 2H), 2.17 (dt, J = 12.6, 5.7 Hz, 2H), 1.98 (d, J = 25.0 Hz, 1H). | 506.4 (M − 1). | Method 4: RT 9.3 min, P 90% | Example 1H and (3S)-3- methoxy- pyrrolidine; Yield: 8 mg; 0.01 mmol, 9% white amorphous foam |
| 8G | 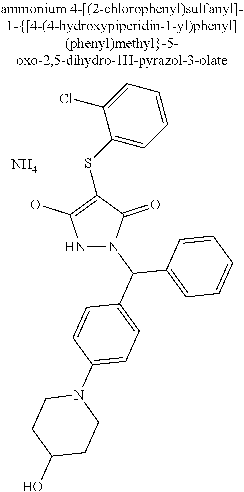 | 1H NMR (400 MHz, MeOD) δ 7.41-7.32 (m, 5H), 7.29-7.24 (m, 2H), 7.21- 7.17 (m, 1H), 7.02-6.97 (m, 2H), 6.97-6.89 (m, 2H), 6.60- 6.56 (m, 1H), 6.48 (s, 1H), 3.82-3.73 (m, 1H), 3.66-3.57 (m, 2H), 3.22- 3.12 (m, 2H), 2.05-1.91 (m, 2H), 1.75-1.63 (m, 2H), 1.31 (s, 1H). | 509.9 (M + 1). | Method 4: RT 6.6 min, P 80% | Example 1H and 4- hydroxy- piperidine; Yield: 5 mg; 0.01 mmol, 5% white solid. |
| 8H | 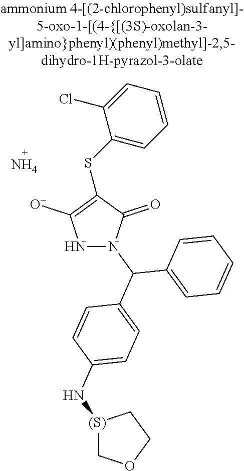 | 1H NMR (400 MHz, MeOD) δ 7.42-7.27 (m, 26H), 7.21- 7.13 (m, 10H), 6.99-6.88 (m, 8H), 6.68-6.62 (m, 6H), 6.61- 6.55 (m, 5H), 6.43 (s, 3H), 4.11 (ddd, J = 10.5, 7.1, 3.6 Hz, 3H), 3.98 (ddd, J = 14.9, 8.3, 4.9 Hz, 6H), 3.86 (td, J = 8.2, 5.3 Hz, 3H), 3.69 (dd, J = 8.8, 3.3 Hz, 3H), 2.33-2.22 (m, 3H), 1.96-1.86 (m, 3H). | 492.05 (M − 1) | Method 4: RT 7.5 min, P 80% | Example 1H and (3S)- oxolan-3- amine; Yield: 23 mg; 0.04 mmol, 26% white solid. |
| 8I | 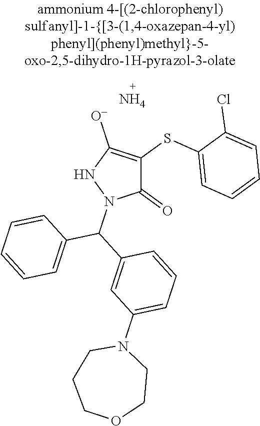 | — | 508.3 (M + 1). | Method 3: RT 11.2 min, P 90% | Example 1I and homo- morpholine; Yield: 4 mg; 0.01 mmol, 5% white powder |
| 8J | 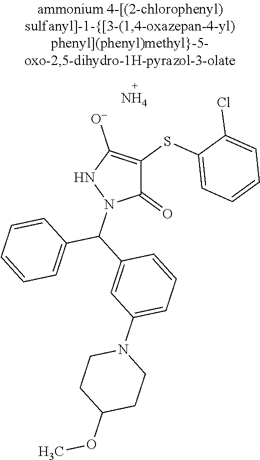 | | 522.8 (M + 1). | Method 4: RT 8.5 min, P 90%, | Example 11 and 4- methoxy piperidine; Yield: 4 mg; 0.01 mmol, 5% beige solid. |
| 8K | 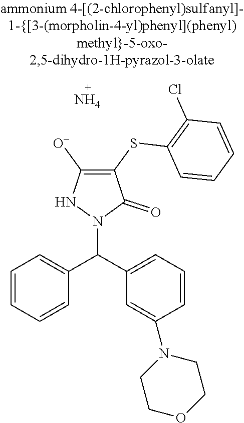 | 1H NMR (400 MHz, D2O) δ 7.37 (t, J = 19.8 Hz, 7H), 7.22- 7.15 (m, 2H), 7.11 (s, 2H), 6.85 (td, J = 7.8, 1.5 Hz, 1H), 6.66 (t, J = 7.4 Hz, 1H), 5.90 (s, 1H), 3.81-3.76 (m, 4H), 3.10- 3.04 (m, 4H). | 492.5 (M − 1). | Method 4: RT 7.4 min, P 98%, | Example 1I and morpholine; Yield: 8 mg; 0.02 mmol, 12% white solid. |
| 8L | 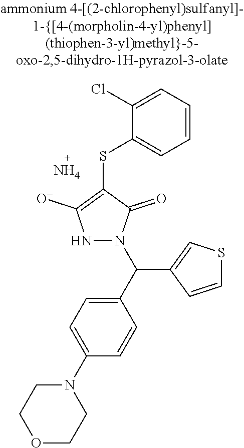 | 1H NMR (400 MHz, D2O) δ 7.42 (dd, J = 5.0, 3.0 Hz, 1H), 7.42 (dd, J = 5.0, 3.0 Hz, 1H), 7.33 (d, J = 8.6 Hz, 2H), 7.33 (d, J = 8.6 Hz, 2H), 7.28-7.20 (m, 2H), 7.27-7.21 (m, 2H), 7.11- 7.01 (m, 3H), 7.09-7.03 (m, 3H), 6.99-6.92 (m, 2H), 7.01- 6.89 (m, 2H), 6.32 (s, 1H), 6.32 (s, 1H), 6.31-6.25 (m, 1H), 6.31-6.20 (m, 1H), 3.88- 3.78 (m, 4H), 3.85-3.75 (m, 4H), 3.19-3.09 (m, 4H), 3.17- 3.07 (m, 4H). | 498.1 (M − 1). | Method 3: RT 11.7 min, P 91%, | Example 1C and morpholine; Yield: 6 mg; 0.01 mmol, 37% white amorphous solid. |
| 8M | 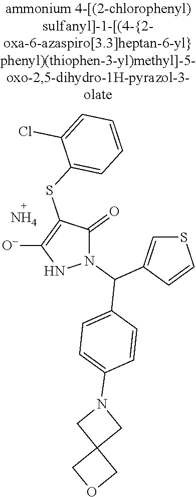 | — | 512.5 (M − 1). | Method 4: RT 7.5 min, P 88%, | Example 1C and 2-oxa- 6- azaspiro[3.3] heptane; Yield: 4 mg; 0.01 mmol, 4% beige solid |
| 8N | 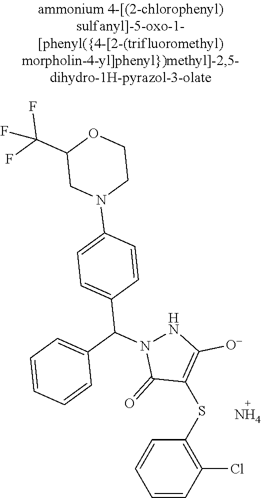 | — | 562.2 (M + 1). | Method 3: RT 14.1 min, P 90%, | Example 1H and 2- (trifluoro- methyl) morpholine; Yield: 3 mg; 0.01 mmol, 4.9% beige solid |
| 8O | 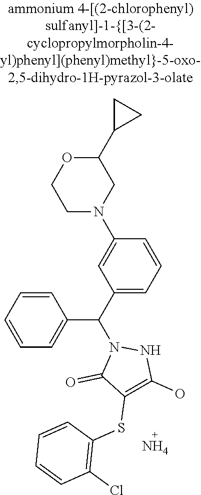 | 1H NMR (400 MHz, D2O) d 7.58-6.94 (m, 9H), 6.84 (t, J = 7.3 Hz, 1H), 6.64 (s, 1H), 5.89 (s, 1H), 3.89 (t, J = 13.7 Hz, 1H), 3.60 (dd, J = 25.7, 12.0 Hz, 2H), 3.36 (d, J = 11.9 Hz, 1H), 2.85- 2.52 (m, 4H), 2.19-1.95 (m, 1H), 0.78 (s, 1H), 0.47 (d, J = 7.9 Hz, 2H), 0.21 (d, J = 13.4 Hz, 2H). | 534.2 (M + 1 | Method 1: RT 3.51 min, P 95%,) | Example 1I and 2- cyclopropyl- morpholine; Yield: 20 mg; 0.03 mmol, 25.1% white solid |
| 8P | 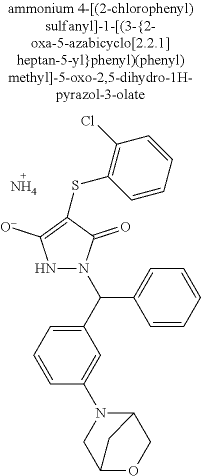 | 1H NMR (400 MHz, MeOD) d 7.67-6.53 (m, 13H), 4.51 (d, J = 25.4 Hz, 2H), 3.94-3.72 (m, 2H), 3.64-3.46 (m, 1H), 3.18- 3.01 (m, 1H), 2.02 (ddd, J = 27.6, 18.2, 11.1 Hz, 2H). | 506.05 (M + 1) | Method 1: RT 3.57 min, P 82%, | Example 1I and 3-{2- oxa-5- azabicyclo [2.2.1] heptane; Yield: 20 mg; 0.03 mmol, 22.8% white flakes. |
| 8Q | 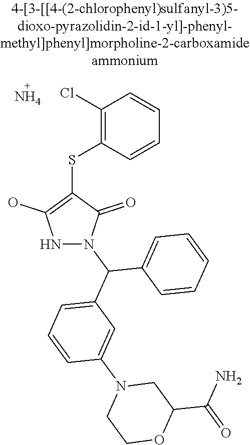 | 1H NMR (400 MHz, MeOD) 7.89-6.85 (m, 11H), 6.78 (td, J = 7.6, 1.6 Hz, 1H), 6.64 (dt, J = 14.0, 3.5 Hz, 1H), 6.31 (dd, J = 9.9, 4.3 Hz, 1H), 4.19-4.00 (m, 2H), 3.96- 3.73 (m, 2H), 3.56-3.42 (m, 1H), 2.83 (td, J = 11.9, 3.4 Hz, 1H), 2.69 (dd, J = 15.1, 7.3 Hz, 1H). | 537.2 (M + 1). | Method 1: RT 2.89 min, P 93%, | Example 1I and morpholine- 2- carboxamide; Yield: 20 mg; 0.03 mmol, 17.7% white flakes |
| 8R | 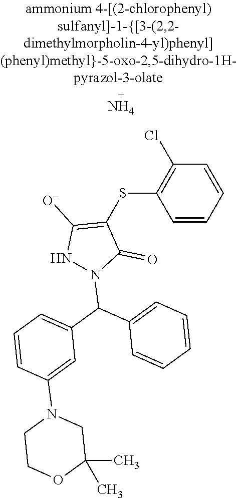 | 1H NMR (400 MHz, MeOD) 7.59-7.19 (m, 10H), 7.08 (dd, J = 7.8, 1.1 Hz, 1H), 7.01-6.95 (m, 1H), 6.77 (td, J = 7.6, 1.5 Hz, 1H), 6.69- 6.63 (m, 1H), 6.33 (d, J = 7.9 Hz, 1H), 3.86- 3.83 (m, 2H), 3.13-3.08 (m, 2H), 3.01-2.96 (m, 2H), 1.30 (s, 6H). | 522.2 (M + 1) | Method 1: RT 3.51 min, P 100% | Example 1I and 2,2- dimethyl- morpholine; Yield: 2 mg; 0.01 mmol, 4.3% beige solid |
| 8S | 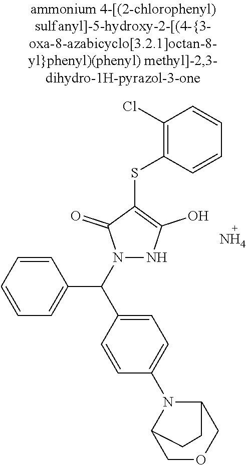 | 1H NMR (400 MHz, MeOD) 7.43-7.31 (m, 6H), 7.27-7.23 (m, 2H), 7.21- 7.17 (m, 1H), 7.00-6.85 (m, 4H), 6.65-6.59 (m, 1H), 6.47 (s, 1H), 4.19-4.13 (m, 2H), 3.92 (d, J = 10.7 Hz, 2H), 3.54 (d, J = 10.6 Hz, 2H), 2.08-1.99 (m, 4H). | 518.2 (M − 1) | Method 1: RT 3.26 min, P 87.8% | Example 1H and 3-oxa-8- azabicyclo [3.2.1]octane; Yield: 8 mg; 0.01 mmol, 7.8% beige solid |
| 8T | 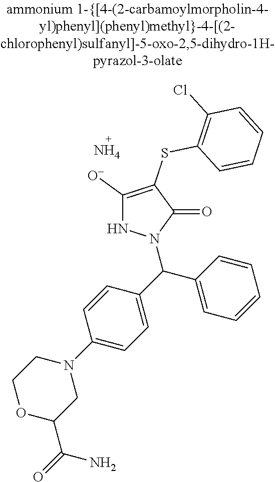 | — | 535.1 (M − 1) | Method 1: RT 2.87 min, P 86.4% | Example 1H and morpholine- 2- carboxamide; Yield: 2.5 mg; 0.01 mmol, 2.9% beige solid |
| 8U | 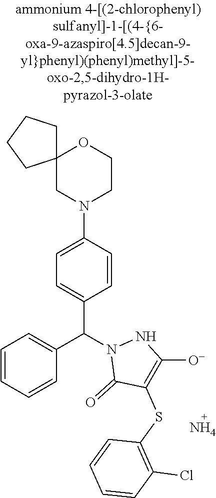 | 1H NMR (400 MHz, MeOD) 7.67-7.26 (m, 10H), 7.08 (dd, J = 7.8, 1.1 Hz, 1H), 7.01-6.88 (m, 3H), 6.77 (td, J = 7.6, 1.4 Hz, 1H), 6.65 (s, 1H), 6.30 (s, 1H), 3.91-3.82 (m, J = 3.4 Hz, 3H), 3.21-3.01 (m, 6H), 1.93- 1.56 (m, 14H). | 546.8 (M − 1) | Method 4: RT 11 min, P 92.6%, | Example 1H and 6-oxa-9- azaspiro[4.5] decane; Yield: 6 mg; 0.01 mmol, 7.3% yellow powder |
| 8V | 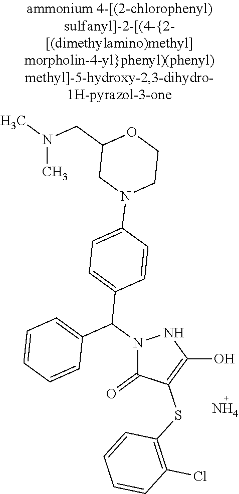 | 1H NMR (400 MHz, DMSO) 7.32-7.18 (m, 7H), 7.13 (d, J = 8.6 Hz, 2H), 6.99-6.89 (m, 2H), 6.86 (d, J = 8.8 Hz, 2H), 6.57-6.52 (m, 1H), 6.28 (s, 1H), 3.99-3.45 (m, 7H), 2.67 (dd, J = 16.4, 8.0 Hz, 2H), 2.38 (s, 6H). | 551.2 (M + 1) | Method 3- 30): RT 4.3 min, P 95.6% | Example 1H and dimethyl [(morpholin- 2-yl)methyl] amine; Yield: 8 mg; 0.01 mmol, 14.3% beige solid |
| 8W | 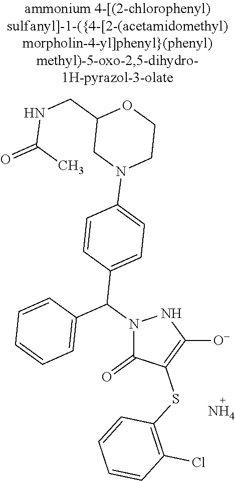 | — | 563.4 (M − 1) | Method 1: RT 6.6 min, P 93.2% | Example 1H and N- [(morpholin- 2- yl)methyl] acetamide; Yield: 5 mg; 0.01 mmol, 5.3% yellow powder. |
| 8X | 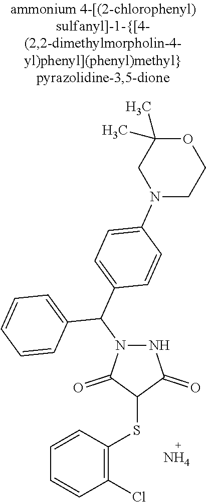 | — | 521.7 (M − 1) | Method 4: RT 9.5 min, P 97.2% | Example 1H and 2,2- dimethyl- morpholine; Yield: 3 mg; 0.01 mmol, 5.7% beige solid |
| 8Y | 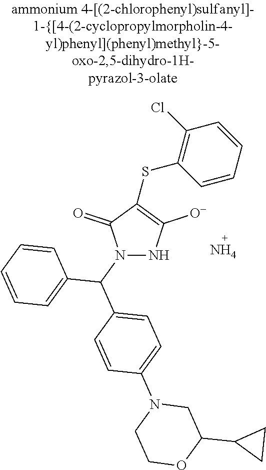 | 1H NMR (400 MHz, MeOD) 7.54-7.05 (m, 7H), 7.05-6.87 (m, 3H), 6.61- 6.54 (m, 1H), 6.49 (s, 1H), 4.06-3.98 (m, 1H), 3.79-3.64 (m, 2H), 3.50 (ddd, J = 8.1, 4.3, 1.2 Hz, 1H), 3.20-2.89 (m, 2H), 2.81 (tdd, J = 12.1, 3.3, 1.4 Hz, 1H), 2.71- 2.62 (m, 1H), 1.02-0.84 (m, 1H), 0.63-0.52 (m, 2H), 0.49- 0.28 (m, 2H). | 532.2 (M − 1) | Method 1: RT 3.49 min, P 100% | Example 1H and 2- cyclopropyl- morpholine; Yield: 6 mg; 0.01 mmol, 7.4% yellow powder |
| 8Z | 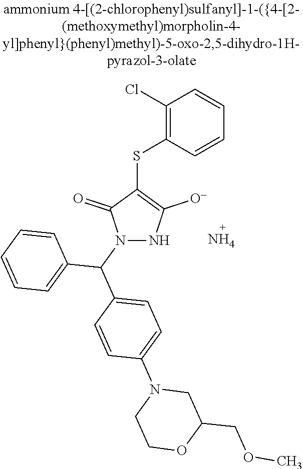 | 1H NMR (400 MHz, MeOD) 7.71-7.14 (m, 8H), 7.08 (dd, J = 7.8 Hz, 1H), 7.03-6.91 (m, 2H), 6.78 (t, J = 7.9 Hz, 1H), 6.64 (s, 1H), 6.30 (s, 1H), 4.03 (d, J = 10.7 Hz, 1H), 3.89- 3.74 (m, 2H), 3.62 (d, J = 11.2 Hz, 1H), 3.57- 3.49 (m, 3H), 3.44-3.39 (m, 3H), 2.81 (m, 1H), 2.58 (m, 1H). | 536.1 (M − 1) | Method 1: RT 3.23 min, P 89.7% | Example 1H and 2- (methoxy- methyl) morpholine; Yield: 18 mg; 0.03 mmol, 21.6% yellow powder. |
| 8AA | 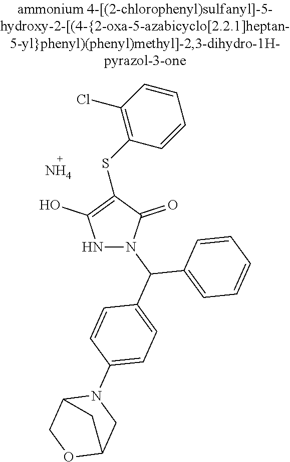 | — | 504.4 (M − 1) | Method 1: RT 3.19 min, P 88% | Example 1H and 2-oxa-5- azabicyclo [2.2.1]heptane; Yield: 3 mg; 0.01 mmol, 3.7% yellow powder |
| 8AB | 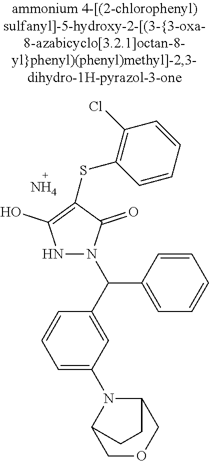 | 1H NMR (400 MHz, MeOD) 7.54 (m, 2H), 7.43-7.21 (m, 5H), 7.19-7.11 (m, 1H), 7.08 (dd, J = 7.8, 1.3 Hz, 1H), 6.99- 6.85 (m, 2H), 6.77 (td, J = 7.6, 1.6 Hz, 1H), 6.69-6.64 (m, 1H), 6.36 (dd, J = 8.0, 1.5 Hz, 1H), 4.14 (s, 2H), 3.87 (d, J = 10.8 Hz, 2H), 3.48 (d, J = 10.9 Hz, 2H), 2.02- 1.94 (m, 4H) | 518.2 (M − 1) | Method 1: RT 4.92 min, P 93.3% | Example 1I and 3-oxa-8- azabicyclo [3.2.1]octane; Yield: 10 mg; 0.02 mmol, 8.4% white flakes |
| 8AC | 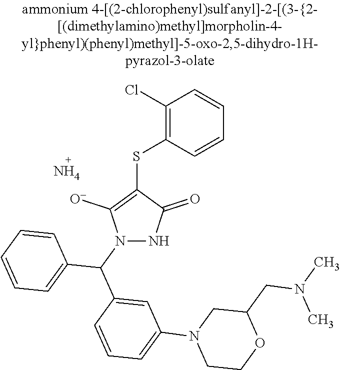 | 1H NMR (400 MHz, MeOD) 7.60-7.22 (m, 8H), 7.09 (d, J = 7.8 Hz, 1H), 7.05-6.99 (m, 1H), 6.78 (t, J = 7.2 Hz, 1H), 6.66 (t, J = 7.4 Hz, 1H), 6.36- 6.28 (m, 1H), 4.01-3.94 (m, 1H), 3.89-3.48 (m, 6H), 2.84- 2.75 (m, 2H), 2.40 (s, 6H). | 552 (M + 1) | Method 1: RT 3.35 min, P 95.1% | Example 1I and dimethyl [(morpholin-2- yl)methyl] amine; Yield: 15 mg; 0.02 mmol, 12.9% yellow powder |
| 8AD | 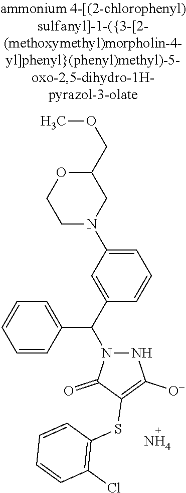 | 1H NMR (400 MHz, MeOD) 7.70-6.91 (m, 11H), 6.78 (td, J = 7.6, 1.6 Hz, 1H), 6.69-6.60 (m, 1H), 6.34 (d, J = 6.8 Hz, 1H), 3.99 (dd, J = 11.4, 1.7 Hz, 1H), 3.85-3.70 (m, 2H), 3.64- 3.43 (m, 4H), 3.41-3.36 (m, 3H), 2.78 (td, J = 11.8, 3.4 Hz, 1H), 2.56 (dd, J = 11.8, 10.6 Hz, 1H). | 538.2 (M + 1) | Method 1: RT 3.22 min, P 87% | Example 1I and 2- (methoxy- methyl) morpholine; Yield: 10 mg; 0.02 mmol, 8.2% white flakes |
| 8AE | 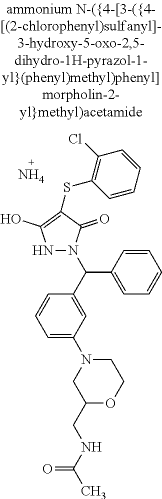 | 1H NMR (400 MHz, DMSO) 8.03-7.95 (m, J = 5.5 Hz, 1H), 7.34-7.20 (m, 8H), 7.19-7.12 (m, J = 7.9 Hz, 1H), 6.99-6.90 (m, 3H), 6.88- 6.80 (m, J = 8.1 Hz, 1H), 6.72- 6.68 (m, J = 7.5 Hz, 1H), 6.60- 6.51 (m, J = 8.5, 4.5 Hz, 1H), 6.30 (s, 1H), 3.91 (d, J = 13.9 Hz, 1H), 3.62- 3.51 (m, J = 30.4, 12.0 Hz, 3H), 3.47-3.38 (m, 1H), 3.22- 3.13 (m, J = 5.8 Hz, 2H), 2.71- 2.60 (m, 1H), 2.41-2.32 (m, J = 20.7, 11.1 Hz, 1H), 1.85-1.79 (m, J = 1.4 Hz, 3H). | 565.4 (M + 1) | Method 3- 30): RT 10.7 min, P 85.6% | Example 1I and N- [(morpholin- 2- yl)methyl] acetamide; Yield: 10 mg; 0.02 mmol, 8.9% white solid. |
| 8AF | 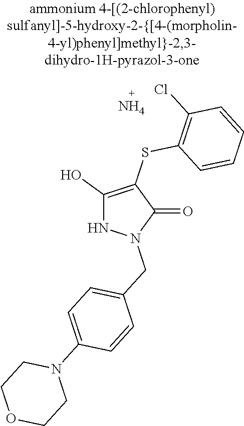 | 1H NMR (400 MHz, D2O) δ 7.33-7.26 (m, 3H), 7.14 (d, J = 8.7 Hz, 2H), 7.05-6.99 (m, 2H), 6.59-6.55 (m, 1H), 4.61 (s, 2H), 3.92-3.85 (m, 4H), 3.28- 3.21 (m, 4H). | M + 1 418.0 | Method 3: RT 6.1 min, P 98.8% | Example 9V and morpholine; Yield: 8.1 mg; 0.02 mmol, 13.7% white solid. |
| 8AG | 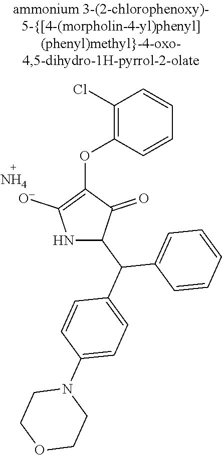 | 1H NMR (400 MHz, MeOD) δ 7.38-7.33 (m, 2H), 7.30-7.22 (m, 5H), 7.10 (dd, J = 7.8, 1.4 Hz, 1H), 6.97- 6.91 (m, 2H), 6.83 (td, J = 7.5, 1.7 Hz, 1H), 6.77 (td, J = 7.6, 1.4 Hz, 1H), 5.89 (d, J = 7.8 Hz, 1H), 4.69 (d, J = 2.5 Hz, 1H), 4.56 (d, J = 2.6 Hz, 1H), 3.85- 3.81 (m, 4H), 3.15-3.10 (m, 4H). | M + 1 493.4 | Method 3: RT 11.6 min, P 91.4% | Example 6G and morpholine; Yield: 16.3 mg; 0.03 mmol, 7.8, beige- yellow solid. |

### Example 9A

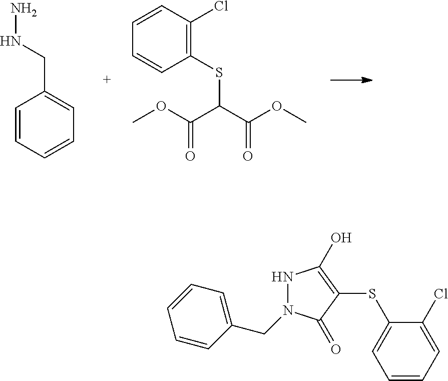 [SMILES: NNCc1ccccc1 ; COC(=O)C(Sc1ccccc1Cl)C(=O)OC ; O=c1c(Sc2ccccc2Cl)c(O)[nH]n1Cc1ccccc1]

### 4-[(2-chlorophenyl)sulfanyl]-5-hydroxy-2,3-dihydro-1H-pyrazol-3-one

Benzylhydrazine (1.17 g; 4.91 mmol; 1.00 eq.) and 2-(2-Chlorophenylsulfanyl)-malonic acid dimethyl ester (Intermediate 4A) (1.67 g; 5.90 mmol; 1.20 eq.) are dissolved in [1,4]-Dioxane (12.00 ml) and heated under microwave irradiation at 160° C. for 30 min. Then the RM is diluted with AcOEt/hexane (1:1) mixture and extracted with 2% aq ammonia. The aqueous extract is washed with mixture of AcOEt/Hexane, evaporated and purified by prep. HPLC (C-18, Water/0.01% NH3-ACN/0.01% NH3) to obtain 2-benzyl-4-[(2-chlorophenyl)sulfanyl]-5-hydroxy-2,3-dihydro-1H-pyrazol-3-one (0.75 g; 1.62 mmol; 33%) Method 5, RT 18.1 min, P 94%, (M+1) 333.4, (M−1) 331.4. 1H NMR (400 MHz, DMSO) δ 11.69 (bs, 1H), 10.40 (bs, 1H), 7.48-7.33 (m, 3H), 7.33-7.26 (m, 1H), 7.26-7.17 (m, 3H), 7.15-7.04 (m, 1H), 6.71 (dd, J=7.9, 1.5 Hz, 1H), 4.88 (s, 2H).

The following compounds are prepared by the procedure for Example 9A, using the appropriate starting materials:

| Ex. | | | | | Starting |
|---|---|---|---|---|---|
| No. | Name and structure | NMR | mz | RT | materials |
| 9B | 3-({4-[(2-chlorophenyl)sulfanyl]-3- | 1H NMR (400 | M − 1 | Method | Intermediate 4A and |
| | hydroxy-4-oxo-2,5-dihydro-1H-pyrazol-1- | MHz, D2O) δ | 355.9 | 3: RT | hydrazine; |
| | yl}methyl)benzonitrile | 7.67 (dd, J = | | 8.9 min, | Yield: 98.9 mg; |
| | | 7.7, 1.3 Hz, 1H), | | p 98.7%. | 0.27 mmol, 40, white |
| | 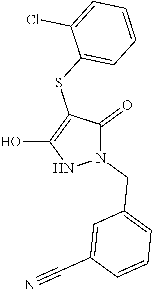 [SMILES: N#Cc1cccc(Cn2[nH]c(O)c(Sc3ccccc3Cl)c2=O)c1] | 7.63 (s, 1H), 7.57 (d, J = 7.9 Hz, 1H), 7.47 (t, J = 7.8 Hz, 1H), 7.30-7.21 (m, 1H), 7.05-6.96 (m, 2H), 6.48- 6.39 (m, 1H), 4.65 (s, 2H). | | | solid. |
| 9C | 4-[(2-chlorophenyl)sulfanyl]-5-hydroxy-2- | 1H NMR (400 | M + 1 | Method | Intermediate 4A and |
| | [(3-methylphenyl)methyl]-2,3-dihydro- | MHz, D2O) 7.36- | 347.30 | 5: : RT | hydrazine; |
| | 1H-pyrazol-3-one | 7.27 (m, 2H), | | 19.1 | Yield: 30.0 mg; |
| | | 7.25-7.19 (m, | | min, p | 0.08 mmol, 3% |
| | 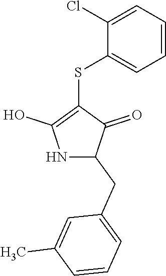 [SMILES: Cc1cccc(CC2NC(O)=C(Sc3ccccc3Cl)C2=O)c1] | 1H), 7.19-7.04 (m, 4H), 6.62- 6.56 (m, 1H), 4.67 (s, 2H), 2.31 (s, 3H) | | 97.4% | beige solid. |
| 9D | 4-[(2-chlorophenyl)sulfanyl]-5-hydroxy-2- | 1H NMR (400 | M + 1 | Method | Intermediate 4A and |
| | {[2-(trifluoromethoxy)phenyl]methyl}-2,3- | MHz, D2O) 7.43- | 417.2 | 3: RT | hydrazine; |
| | dihydro-1H-pyrazol-3-one | 7.24 (m, 5H), | | 12.7 | Yield: 135.20 mg; |
| | | 7.03 (dtd, J = | | min, p | 0.30 mmol, 19.7% |
| | 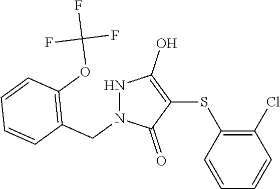 [SMILES: O=c1c(Sc2ccccc2Cl)c(O)[nH]n1Cc1ccccc1OC(F)(F)F] | 13.4, 7.6, 6.1 Hz, 2H), 6.64 (dd, J = 7.8, 1.6 Hz, 1H), 4.76 (s, 2H). | | 97.6% | cream powder. |
| 9E | 2-[(2H-1,3-benzoidioxol-5-yl)methyl]-4- | 1H NMR (400 | M + 1 | Method | Intermediate 4A and |
| | [(2-chlorophenyl)sulfanyl]-5-hydroxy-2,3- | MHz, D2O) 7.39- | 377.1 | 5: RT | hydrazine; |
| | dihydro-1H-pyrazol-3-one | 7.32 (m, 1H), | | 17.8 | Yield: 36.1 mg; |
| | | 7.15-7.05 (m, | | min, p | 0.09 mmol, 18.1%, |
| | 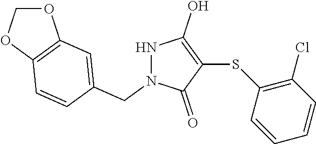 [SMILES: O=c1c(Sc2ccccc2Cl)c(O)[nH]n1Cc1ccc2c(c1)OCO2] | 2H), 6.93-6.85 (m, 3H), 6.61- 6.55 (m, 1H), 5.98 (s, 2H), 4.62 (s, 2H) | | 99.1%. | white solid. |
| 9F | 4-[(2-chlorophenyl)sulfanyl]-5-hydroxy-2- | 1H NMR (400 | M − 1 | Method | Intermediate 4A and |
| | [(naphthalen-1-yl)methyl]-2,3-dihydro- | MHz, D2O) δ | 381.7 | 3: RT | hydrazine; |
| | 1H-pyrazol-3-one | 8.16-8.09 (m, | | 11.8 | Yield: 42 mg; 0.10 |
| | | 1H), 7.94-7.89 | | min, p | mmol, 21.8%, white |
| | 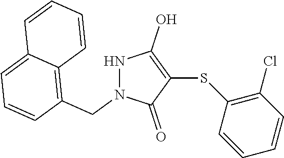 [SMILES: O=c1c(Sc2ccccc2Cl)c(O)[nH]n1Cc1cccc2ccccc12] | (m, 1H), 7.87 (dd, J = 7.4, 1.6 Hz, 1H), 7.57- 7.39 (m, 4H), 7.25 (dt, J = 7.4, 3.6 Hz, 1H), 6.98 (dd, J = 5.9, 3.5 Hz, 2H), 6.64-6.58 (m, 1H), 5.11 (s, 2H). | | 94.6%. | solid. |
| 9G | methyl 3-({4-[(2-chlorophenyl)sulfanyl]-3- | 1H NMR (400 | M − 1 | Method | Intermediate 4A and |
| | hydroxy-5-oxo-2,5-dihydro-1H-pyrazol-1- | MHz, D2O) δ | 355.9 | 3: RT | hydrazine; |
| | yl}methyl)benzoate | 7.94 (dd, J = | | 9.3 min, | Yield: 11 mg; |
| | | 5.9, 4.5 Hz, 1H), | | p 91.8%. | 0.02 mmol, 3.3, white |
| | 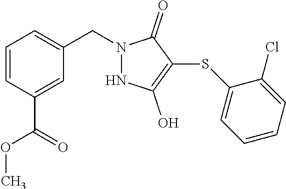 [SMILES: COC(=O)c1cccc(Cn2[nH]c(O)c(Sc3ccccc3Cl)c2=O)c1] | 7.91 (s, 1H), 7.55 (d, J = 7.7 Hz, 1H), 7.46 (t, J = 7.7 Hz, 1H), 7.27 (dd, J = 7.8, 1.3 Hz, 1H), 6.99 (td, J = 7.6, 1.6 Hz, 1H), 6.92 (td, J = 7.7, 1.4 Hz, 1H), 6.38 (dd, J = 7.9, 1.6 Hz, 1H), 3.81 (s, 3H). | | | solid. |
| 9H | 4-({4-[(2-chlorophenyl)sulfanyl]-3- | 1H NMR (400 | M + 1 | Method | Intermediate 4A and |
| | hydroxy-5-oxo-2,5-dihydro-1H-pyrazol-1- | MHz, D2O) δ | 358.30 | 5: : RT | hydrazine; |
| | yl}methyl)benzonitrile | 7.81 (d, J = 8.2 | | 18.4 | Yield: 14.2 mg; |
| | | Hz, 2H), 7.52 (d, | | min, p | 0.04 mmol, 3.5, white |
| | 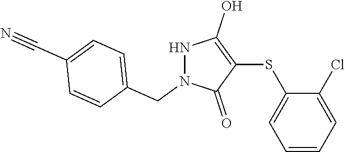 [SMILES: N#Cc1ccc(Cn2[nH]c(O)c(Sc3ccccc3Cl)c2=O)cc1] | J = 8.1 Hz, 2H), 7.43-7.36 (m, 1H), 7.19-7.10 (m, 2H), 6.67- 6.60 (m, 1H) | | 97.9%. | powder.. |
| 9I | 4-[(2-chlorophenyl)sulfanyl]-5-hydroxy-2- | 1H NMR (400 | M + 1 | Method | Intermediate 4A and |
| | [(4-hydroxyphenyl)methyl]-2,3-dihydro- | MHz, D2O) δ | 350.7, | 5: : RT | hydrazine; |
| | 1H-pyrazol-3-one | 7.41-7.34 (m, | M − 1 | 15.0 | Yield: 1.5 mg; |
| | | 1H), 7.29 (d, J = | 348.1 | min, p | 0.004 mmol, 7.4%, |
| | 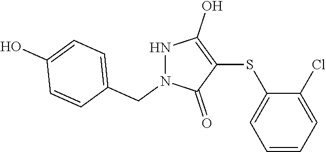 [SMILES: O=c1c(Sc2ccccc2Cl)c(O)[nH]n1Cc1ccc(O)cc1] | 8.4 Hz, 2H), 7.15-7.06 (m, 2H), 6.91 (d, J = 8.5 Hz, 2H), 6.54-6.45 (m, 1H), 4.66 (s, 2H) | | 98.5%. | white solid. |
| 9J | 3-({4-[(2-chlorophenyl)sulfanyl]-3- | Not determined | M − 1 | Method | Intermediate 4A and |
| | hydroxy-5-oxo-2,5-dihydro-1H-pyrazol-1- | | 355.9 | 5: : RT | hydrazine; |
| | yl}methyl)benzoic acid | | | 15.8 | Yield: 4.1 mg; |
| | | | | min, p | 0.01 mmol, 92.7, |
| | 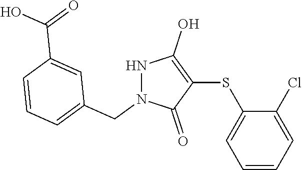 [SMILES: O=C(O)c1cccc(Cn2[nH]c(O)c(Sc3ccccc3Cl)c2=O)c1] | | | 95.9%. | white solid. |
| 9K | 4-[(2-chlorophenyl)sulfanyl]-2-[(5- | 1H NMR (400 | M + 1 | Method | Intermediate 4A and |
| | chlorothiophen-2-yl)methyl]-5-hydroxy- | MHz, D2O) δ | 372.8, | 3: RT | hydrazine; |
| | 2,3-dihydro-1H-pyrazol-3-one | 7.28-7.24 (m, | M − 1 | 11.6 | Yield: 53.4 mg; |
| | | 1H), 7.05-6.96 | 370.9 | min, p | 0.14 mmol, 14.9% |
| | 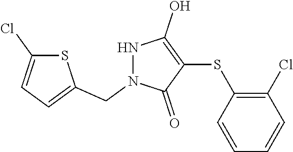 [SMILES: O=c1c(Sc2ccccc2Cl)c(O)[nH]n1Cc1ccc(Cl)s1] | (m, 2H), 6.83 (dd, J = 12.7, 3.8 Hz, 2H), 6.34-6.27 (m, 1H), 4.64 (s, 2H). | | 95.7%. | cream powder. |
| 9L | 4-[(2-chlorophenyl)sulfanyl]-5-hydroxy-2- | 1H NMR (400 | M + 1 | Method | Intermediate 4A and |
| | [(thiophen-3-yl)methyl]-2,3-dihydro-1H- | MHz, D2O) δ | 338.9, | 3: RT | hydrazine; |
| | pyrazol-3-one | 7.40-7.35 (m, | M − 1 | 8.2 min, | Yield: 28.0 mg; 0.08 |
| | | 1H), 7.30-7.24 | 336.9 | p 94.4%. | mmol, 16.7%, white |
| | 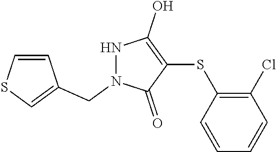 [SMILES: O=c1c(Sc2ccccc2Cl)c(O)[nH]n1Cc1ccsc1] | (M, 2H), 7.09- 6.97 (m, 3H), 6.47 (dd, J = 7.8, 1.6 Hz, 1H), 4.62 (s, 2H). | | | powder. |
| 9M | 4-[(2-chlorophenyl)sulfanyl]-5-hydroxy-2- | 1H NMR (400 | M + 1 | Method | Intermediate 4A and |
| | [(thiophen-2-yl)methyl]-2,3-dihydro-1H- | MHz, D2O) δ | 338.8, | 3: RT | hydrazine; |
| | pyrazol-3-one | 7.34 (dd, J = | M − 1 | 8.3 min, | Yield: 79.9 mg; 0.23 |
| | | 5.1, 1.2 Hz, 1H), | 336.9 | p 98.6%. | mmol, 71.5%, bright |
| | 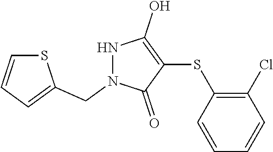 [SMILES: O=c1c(Sc2ccccc2Cl)c(O)[nH]n1Cc1cccs1] | 7.27-7.23 (m, 1H), 7.04-7.01 (m, 1H), 7.00- 6.96 (m, 3H), 6.39-6.35 (m, 1H), 4.76 (s, 2H). | | | yellow powder. |
| 9N | 4-[(2-chlorophenyl)sulfanyl]-5-hydroxy-2- | 1H NMR (400 | M + 1 | Method | Intermediate 4A and |
| | [(4-methoxyphenyl)methyl]-2,3-dihydro- | MHz, DMSO) δ | 363.8 | 3: RT | hydrazine; |
| | 1H-pyrazol-3-one | 7.40 (d, J = 7.8 | | 8.7 | Yield: 55.8 mg; 0.15 |
| | | Hz, 1H), 7.19 | | min, p | mmol, 21%, white |
| | 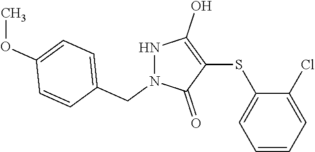 [SMILES: COc1ccc(Cn2[nH]c(O)c(Sc3ccccc3Cl)c2=O)cc1] | (dd, J = 14.9, 8.0 Hz, 3H), 7.11 (t, J = 7.4 Hz, 1H), 6.92 (d, J = 8.4 Hz, 2H), 6.67 (d, J = 7.7 Hz, 1H), 4.79 (s, 2H), 3.74 (s, 3H). | | 98.7%. | powder. |
| 9O | 4-[(2-chlorophenyl)sulfanyl]-5-hydroxy-2- | 1H NMR (400 | M + 1 | Method | Intermediate 4A and |
| | [(3-methoxyphenyl)methyl]-2,3-dihydro- | MHz, D2O) δ | 364.1, | 5: : RT | hydrazine; |
| | 1H-pyrazol-3-one | 7.31-7.20 (m, | M − 1 | 18.3 | Yield: 22.1 mg; 0.06 |
| | | 2H), 7.04-6.94 | 351.4 | min, p | mmol, 7.3%, white |
| | 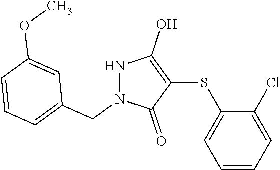 [SMILES: COc1cccc(Cn2[nH]c(O)c(Sc3ccccc3Cl)c2=O)c1] | (m, 2H), 6.92- 6.83 (m, 3H), 6.53-6.45 (m, 1H), 4.60 (s, 2H), 3.70 (s, 3H) | | 100%. | solid. |
| 9P | 4-[(2-chlorophenyl)sulfanyl]-5-hydroxy-2- | 1H NMR (400 | M + 1 | Method | Intermediate 4a and |
| | [(2-methoxyphenyl)methyl]-2,3-dihydro- | MHz, D2O) δ | 364.10, | 5: : RT | hydrazine; |
| | 1H-pyrazol-3-one | 7.43-7.34 (m, | M − 1 | 18.1 | Yield: 7.0 mg; |
| | | 2H), 7.25 (dd, J = | 361.30 | min, p | 0.02 mmol, 1.9%, |
| | 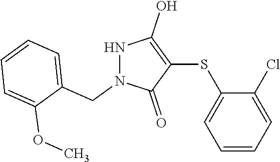 [SMILES: COc1ccccc1Cn1[nH]c(O)c(Sc2ccccc2Cl)c1=O] | 7.5, 1.6 Hz, 1H), 7.16 (td, J = 7.6, 1.4 Hz, 1H), 7.13-7.06 (m, 2H), 7.03 (td, J = 7.4, 0.9 Hz, 1H), 6.78 (dd, J = 7.9, 1.6 Hz, 1H), 4.77 (s, 2H), 3.86 (s, 3H) | | 94.2%. | white solid. |
| 9Q | 4-[(2-chlorophenyl)sulfanyl]-5-hydroxy-2- | 1H NMR (400 | M + 1 | Method | Intermediate 4A and |
| | [(pyridin-4-yl)methyl]-2,3-dihydro-1H- | MHz, DMSO) δ | 334.6, | 3: RT | hydrazine; |
| | pyrazol-3-one | 8.53 (d, J = 3.8 | M − 1 | 2.9 min, | Yield: 10.0 mg; |
| | | Hz, 1H), 8.49 (d, | 332.3 | p 97.1%. | 0.06 mmol, 7.6 beige |
| | 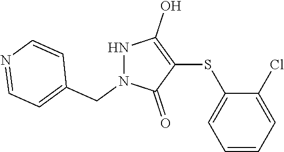 [SMILES: O=c1c(Sc2ccccc2Cl)c(O)[nH]n1Cc1ccncc1] | J = 0.5 Hz, 1H), 7.66 (d, J = 7.8 Hz, 1H), 7.43 (dd, J = 7.8, 4.9 Hz, 1H), 7.39 (dd, J = 7.7, 0.7 Hz, 1H), 7.19 (t, J = 7.5 Hz, 1H), 7.11 (td, J = 7.5, 1.2 Hz, 1H), | | | amorphous solid. |
| | | 6.66 (d, J = 7.6 | | | |
| | | Hz, 1H), 4.90 (s, | | | |
| | | 1H), 3.37 (s, 1H) | | | |
| 9R | 4-[(2-chlorophenyl)sulfanyl]-5-hydroxy-2- | 1H NMR (400 | M + 1 | Method | Intermediate 4A and |
| | [(pyridin-3-yl)methyl]-2,3-dihydro-1H- | MHz, DMSO) ? | 334.9, | 3: RT | hydrazine; |
| | pyrazol-3-one | 8.53 (d, J = 3.8 | M − 1 | 1.2 min, | Yield: 4.0 mg; |
| | | Hz, 1H), 8.49 (d, | 332.2 | p 98.9%. | 0.01 mmol, 1.6, |
| | 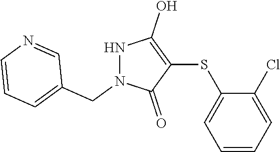 [SMILES: O=c1c(Sc2ccccc2Cl)c(O)[nH]n1Cc1cccnc1] | J = 0.5 Hz, 1H), 7.66 (d, J = 7.8 Hz, 1H), 7.43 (dd, J = 7.8, 4.9 Hz, 1H), 7.39 (dd, J = 7.7, 0.7 Hz, 1H), 7.19 (t, J = 7.5 Hz, 1H), 7.11 (td, J = 7.5, 1.2 Hz, 1H), 6.66 (d, J = 7.6 Hz, 1H), 4.90 (s, 1H), 3.37 (s, 1H) | | | beige amorphous solid |
| 9S | 4-[(2-chlorophenyl)sulfanyl]-5-hydroxy-2- | 1H NMR (400 | M + 1 | Method | Intermediate 4A and |
| | [(pyridin-2-yl)methyl]-2,3-dihydro-1H- | MHz, DMSO) δ | 334.4, | 5: RT | hydrazine; |
| | pyrazol-3-one | 8.53-8.49 (m, | M − 1 | 10.8 | Yield: 31.0 mg; |
| | | 1H), 7.77 (td, J = | 332.2 | min, p | 0.09 mmol, 7.1, white |
| | 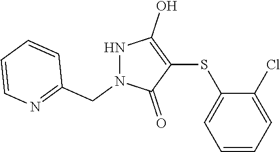 [SMILES: O=c1c(Sc2ccccc2Cl)c(O)[nH]n1Cc1ccccn1] | 7.7, 1.8 Hz, 1H), 7.34 (dd, J = 7.9, 1.1 Hz, 1H), 7.30-7.26 (m, 1H), 7.23-7.17 (m, 2H), 7.06 (td, J = 7.7, 1.5 Hz, 1H), 6.92 (dd, J = 7.8, 0.8 Hz, 1H), 4.76 (s, | | 94.9%. | solid. |
| | | 2H). | | | |
| 9T | 2-[(3-chlorophenyl)methyl]-4-[(2- | 1H NMR (400 | M + 1 | Method | Intermediate 4A and |
| | chlorophenyl)sulfanyl]-5-hydroxy-2,3- | MHz, D2O) δ | 367.4, | 4: RT | hydrazine; |
| | dihydro-1H-pyrazol-3-one | 7.43-7.32 (m, | M − 1 | 6.8 min, | Yield: 12.0 mg; 0.03 |
| | | 4H), 7.30-7.25 | 365.6 | p 97.4%. | mmol, 2.1%, white |
| | 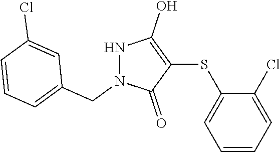 [SMILES: O=c1c(Sc2ccccc2Cl)c(O)[nH]n1Cc1cccc(Cl)c1] | (m, 1H), 7.10 (dtd, J = 20.2, 7.4, 1.6 Hz, 2H), 6.56 (dd, J = 7.8, 1.7 Hz, 1H), 4.69 (s, 2H). | | | solid. |
| 9U | 2-[(2-chlorophenyl)methyl]-4-[(2- | 1H NMR (400 | M + 1 | Method | Intermediate 4A and |
| | chlorophenyl)sulfanyl]-5-hydroxy-2,3- | MHz, DMSO) δ | 367.6, | 5: RT | hydrazine; |
| | dihydro-1H-pyrazol-3-one | 7.51-7.46 (m, | M − 1 | 19.8 | Yield: 32 mg; 0.09 |
| | | 1H), 7.43-7.39 | 365.7 | min, p | mmol, 2.2%, white |
| | 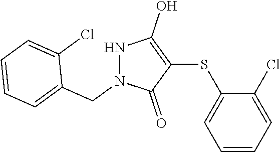 [SMILES: O=c1c(Sc2ccccc2Cl)c(O)[nH]n1Cc1ccccc1Cl] | (m, 1H), 7.36 (ddd, J = 7.2, 4.4, 2.0 Hz, 2H), 7.25 (ddd, J = 7.9, 7.4, 1.3 Hz, 1H), 7.16-7.07 (m, 2H), 6.80 (dd, J = 8.0, 1.5 Hz, 1H), 5.01 (s, 2H). | | 97.7%. | solid. |
| 9V | 2-[(4-chlorophenyl)methyl]-4-[(2- | 1H NMR (400 | M + 1 | Method | Intermediate 4A and |
| | chlorophenyl)sulfanyl]-5-hydroxy-2,3- | MHz, D2O) δ | 368.4, | 3: RT | hydrazine; |
| | dihydro-1H-pyrazol-3-one | 7.34-7.20 (m, | M − 1 | 11.5 | Yield: 21.0 mg; |
| | | 6H), 7.01-6.97 | 366.4 | min, p | 0.08 mmol, 12%, |
| | 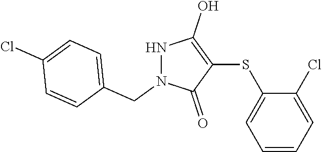 [SMILES: O=c1c(Sc2ccccc2Cl)c(O)[nH]n1Cc1ccc(Cl)cc1] | (m, 2H), 6.41- 6.36 (m, 1H), 4.58 (s, 2H). | | 99.3%. | white solid. |
| 9W | 4-[(2-chlorophenyl)sulfanyl]-5-hydroxy-2- | 1H NMR (400 | M + 1 | Method | Intermediate 4A and |
| | [(3-nitrophenyl)methyl]-2,3-dihydro-1H- | MHz, DMSO) δ | 378.3, | 4: RT | hydrazine; |
| | pyrazol-3-one | 8.15 (ddd, J = | M − 1 | 6.1 min, | Yield: 31 mg; |
| | | 8.1, 2.3, 1.1 Hz, | 375.7 | p 98.1%. | 0.08 mmol, 2.8, white |
| | 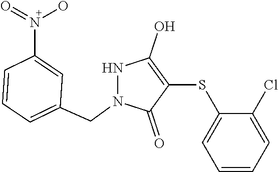 | 1H), 8.09 (s, 1H), 7.78-7.59 (m, 2H), 7.14 (ddd, J = 26.4 25.6, 7.4 Hz, 5H), 6.71 (dd, J = 7.7, 1.6 Hz, 1H), 4.70 (s, 2H). | | | solid. |
| 9X | 4-[(2-chlorophenyl)sulfanyl]-5-hydroxy-2- | 1H NMR (400 | M + 1 | Method | Intermediate 4A and |
| | [(2-nitrophenyl)methyl]-2,3-dihydro-1H- | MHz, D2O) δ | 378.2, | 4: RT | hydrazine; |
| | pyrazol-3-one | 8.00 (dd, J = | M − 1 | 5.7 min, | Yield: 8 mg; |
| | | 8.2, 1.3 Hz, 1H), | 376.2 | p 96.6%. | 0.02 mmol, 1.8%, |
| | 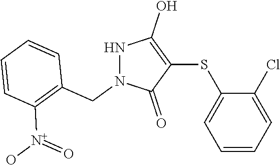 | 7.62 (td, J = 7.6, 1.3 Hz, 1H), 7.52-7.46 (m, 1H), 7.34 (dd, J = 7.8, 1.0 Hz, 1H), 7.26 (dd, J = 7.7, 1.6 Hz, 1H), 7.08-6.97 (m, 2H), 6.53 (dd, J = 7.7, 1.7 Hz, 1H), 5.04 (s, 2H). | | | white powder. |
| 9Y | 4-[(2-chlorophenyl)sulfanyl]-5-hydroxy-2- | 1H NMR (400 | M − 1 | Method | Intermediate 4A and |
| | [(4-nitrophenyl)methyl]-2,3-dihydro-1H- | MHz, D2O) δ | 355.9 | 3: RT | hydrazine; |
| | pyrazol-3-one | 8.17-8.11 (m, | | 10.4 | Yield: 66.3 mg; 0.16 |
| | | 2H), 7.47-7.39 | | min, p | mmol, 15.9%, yellow |
| | 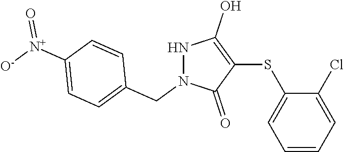 [SMILES: O=c1c(Sc2ccccc2Cl)c(O)[nH]n1Cc1ccc([N+](=O)[O-])cc1] | (m, 2H), 7.27- 7.21 (m, 1H), 7.02-6.94 (m, 2H), 6.55-6.48 (m, 1H), 4.72 (s, 2H). | | 95.8%. | powder. |
| 9Z | 2-benzyl-5-hydroxy-4-[(2- | 1H NMR (400 | M + 1 | Method | Intermediate 4B and |
| | nitrophenyl)sulfanyl]-2,3-dihydro-1H- | MHz, DMSO) δ | 344.1 | 3: RT | benzylhydrazine; |
| | pyrazol-3-one | 8.22 (dd, J = | | 7.5 min, | Yield: 72.0 mg; 0.21 |
| | | 8.3, 1.3 Hz, 1H), | | p 98.0%. | mmol, 26.6 orange |
| | 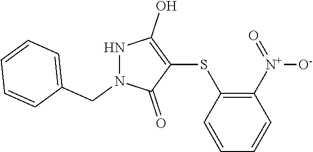 [SMILES: O=c1c(Sc2ccccc2[N+](=O)[O-])c(O)[nH]n1Cc1ccccc1] | 7.61 (t, J = 7.3 Hz, 1H), 7.40- 7.28 (m, 4H), 7.26-7.20 (m, 2H), 7.09 (dd, J = 8.2, 1.2 Hz, 1H), 4.86 (s, 2H). | | | solid. |
| 9AA | 2-benzyl-4-[(5-bromo-2- | 1H NMR (400 | M + 1 | Method | Intermediate 4C and |
| | nitrophenyl)sulfanyl]-5-hydroxy-2,3- | MHz, D2O) δ | 422.9, | 3: RT | benzylhydrazine; Yield: |
| | dihydro-1H-pyrazol-3-one | 8.06 (d, J = 8.8 | M − 1 | 8.9 min, | 4.6 mg; 0.01 |
| | | Hz, 5H), 7.40- | 421.4 | p 99.0%. | mmol, 4.0%, orange |
| | 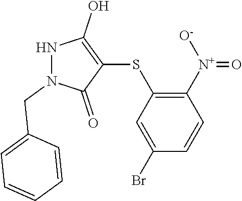 [SMILES: O=c1c(Sc2cc(Br)ccc2[N+](=O)[O-])c(O)[nH]n1Cc1ccccc1] | 7.35 (m, 10H), 7.30 (d, J = 7.5 Hz, 4H), 7.24 (dd, J = 11.3, 4.5 Hz, 12H). | | | solid. |
| 9AB | 2-benzyl-4-[(2-bromophenyl)sulfanyl]-5- | 1H NMR (400 | M + 1 | Method | Intermediate 4D and |
| | hydroxy-2,3-dihydro-1H-pyrazol-3-one | MHz, DMSO) δ | 376.80, | 3: RT | benzylhydrazine; |
| | | 7.43 (dd, J = | M − 1 | 9.0 min, | Yield: 59.0 mg; 0.13 |
| | 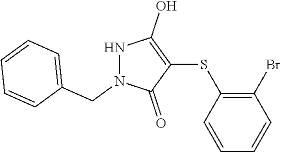 [SMILES: O=c1c(Sc2ccccc2Br)c(O)[nH]n1Cc1ccccc1] | 7.8, 1.2 Hz, 1H), 7.31-7.22 (m, 5H), 7.14-7.08 (m, 1H), 6.94- 6.88 (m, J = 7.5, 1.6 Hz, 1H), 6.76 (dd, J = 7.9, 1.6 Hz, 1H), 4.44 (s, 2H). | 374.90 | p 90.1%. | mmol, 12.5%, yellow powder. |

### Intermediates

### Intermediate 1A

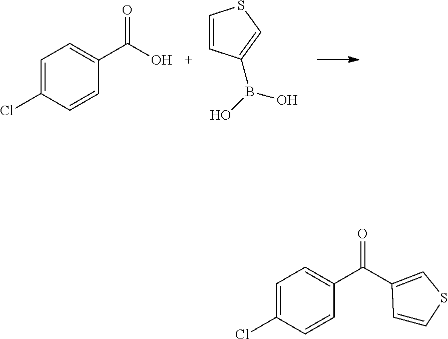 [SMILES: O=C(O)c1ccc(Cl)cc1 ; OB(O)c1ccsc1 ; O=C(c1ccc(Cl)cc1)c1ccsc1]

### (4-chlorophenyl) (thiophen-3-yl)methanone

4-chlorobenzoic acid (5.00 g; 31.94 mmol; 1.00 eq.), 3-thienylboronic acid (4.90 g; 38.32 mmol; 1.20 eq.), dimethyldicarbonate (4.11 ml; 38.32 mmol; 1.20 eq.) are dissolved in anhydrous [1,4]-dioxane (60.00 ml) and the reaction mixture is purged with argon for 10 min, then tetrakis(triphenylphosphine)palladium(0) (1.11 g; 0.96 mmol; 0.03 eq.) is added and the reaction mixture is purged with argon for additional 10 min. Subsequently the RM is heated overnight at 850° C. in an oil bath. After cooling the reaction mixture is filtered through celite and washed with EtOAc. The filtrate is successively washed with 1M HCl, saturated solution of Na₂CO₃ and brine. The organic extract is dried over MgSO₄ and evaporated. The residue is dissolved in the mixture of hexane and AcOEt (9:1) and decolorized with charcoal. The hot solution is filtered and left for crystallization. The precipitate is filtered off, washed with hexane and dried over air to obtain (4-chlorophenyl)(thiophen-3-yl)methanone (4.96 g; 20.78 mmol; Yield: 65%). Method 1: RT 3.81 min, p 98%, M+1 222.9.

The following compounds are prepared by the procedure of Intermediate 1A, using the appropriate starting materials:

| Inter- | | | | |
|---|---|---|---|---|
| mediate | | | | Starting materials |
| No. | Name and structure | mz | RT | and yield |
| 1B | (3-chlorophenyl)(thiophen-3-yl)methanone | M + 1 222.9 | Method | 3-chlorobenzoic |
| | | | 1: 3.81 | acid, 3- |
| | 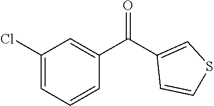 [SMILES: O=C(c1ccsc1)c1cccc(Cl)c1] | | min | thienylboronic acid; Yield: 6.12 g; 27.5 mmol; 87%. |
| 1C | (4-methoxyphenyl)(thiophen-3-yl)methanone | M + 1 218.8 | Method | 4-methoxybenzoic |
| | | | 1: RT | acid, 3- |
| | 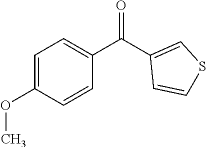 | | 3.34 min, | thienylboronic acid; Yield: 410 mg; 1.43 mmol; 11%. |
| 1D | bis(thiophen-3-yl)methanone | M + 1 194.8. | Method | thiophene-3- |
| | | | 1: RT | carboxylic acid, 3- |
| | 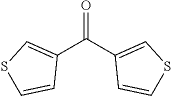 [SMILES: O=C(c1ccsc1)c1ccsc1] | | 3.17 min | thienylboronic acid; Yield: 900 mg; 4.58 mmol; 57%. |
| 1E | 4-[4-(Thiophene-3-carbonyl)phenyl]oxane 405 | M + 1 272.8. | Method | 3-thienylboronic |
| | | | 1: RT | acid and |
| | 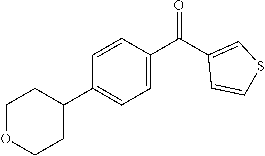 [SMILES: O=C(c1ccc(C2CCOCC2)cc1)c1ccsc1] | | 3.48 min | Intermediate 6A; Yield: 246.50 mg; 0.89 mmol; 57% cream solid |

### Intermediate 2A

### (4-Bromophenyl)-(thiophen-3-yl)methanol

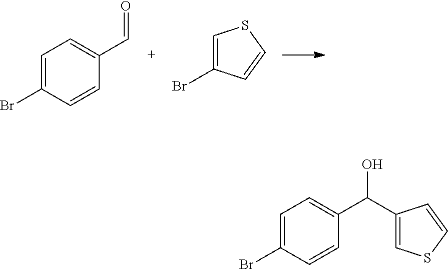 [SMILES: O=Cc1ccc(Br)cc1 ; Brc1ccsc1 ; OC(c1ccc(Br)cc1)c1ccsc1]

3-Bromo-thiophene (2.87 ml; 29.75 mmol; 1.00 eq.) is added to a 1.3M solution of isopropylmagnesium chloride lithium chloride complex solution in THF (22.88 ml; 29.75 mmol; 1.00 eq.) and stirred for 1 hour. Then the solution of 4-Bromobenzaldehyde (6.05 g; 32.72 mmol; 1.10 eq.) in anhydrous THF (20.00 ml) is added slowly. The reaction mixture is slowly warmed up to rt and left stirring overnight at rt. Then the reaction mixture is quenched with sat. NH₄Cl solution and extracted 3 times with AcOEt. The combined organic extracts are washed with brine, dried over Na₂SO₄ and evaporated to oily residue. The crude product is purified by FCC (AcOEt/Hexane 0=>1:9) to obtain ((4-Bromophenyl)-(thiophen-3-yl)methanol (3.61 g; 13.01 mmol; 43.7%; Yellow oil). Method 1: RT 3.15 min, p 99%, M-OH 252.7. ¹H NMR (400 MHz, DMSO) δ 7.54-7.47 (m, 2H), 7.44 (dd, J=5.0, 3.0 Hz, 1H), 7.37-7.26 (m, 3H), 6.97 (dd, J=5.0, 1.2 Hz, 1H), 5.95 (d, J=4.5 Hz, 1H), 5.74 (d, J=4.4 Hz, 1H).

### Intermediate 3A

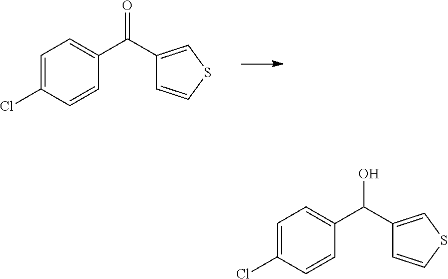 [SMILES: O=C(c1ccc(Cl)cc1)c1ccsc1 ; OC(c1ccc(Cl)cc1)c1ccsc1]

### (4-chlorophenyl) (thiophen-3-yl)methanol

The (4-chlorophenyl)(thiophen-3-yl)methanone (Intermediate 1A) (3.96 g; 16.38 mmol; 1 eq) is dissolved in methanol (50.00 ml) and cooled to 5° C. Subsequently NaBH4 (1.24 g; 32.76 mmol; 2.00 eq.) is added in 10 portions over 30 min. After 1 h when allSM is fully consumed and the reaction mixture is diluted with AcOEt, washed with water, brine, dried over Na₂SO₄ and evaporated to give a dark oil. The residue is purified by FCC (Hexane->AcOEt/Hexane 1:4) to give (4-chlorophenyl)(thiophen-3-yl)methanol (3.38 g, 13.38 mmol; 81.7%; light yellow oil). Method 1: RT 3.52 min, p 99%, M-OH 206.9.

The following compounds are prepared by the procedure for Intermediate 3A, using the appropriate starting materials:

| Inter- | | | | |
|---|---|---|---|---|
| mediate | | | | Starting materials |
| No. | Name and structure | mz | RT | and yield |
| 3B | 3-chlorophenyl)(thiophen-3-yl)methanol | M—OH 206.9 | Method 1: | Intermediate 1B; |
| | | | RT 3.44 min, | Yield: 3.1 g 13.5 |
| | 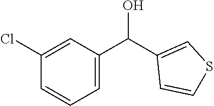 [SMILES: OC(c1ccsc1)c1cccc(Cl)c1] | | | mmol, 49%. |
| 3C | (4-methoxyphenyl)(thiophen-3- | M—OH 202.8 | Method 1: | Intermediate 1C; |
| | yl)methanol | | RT 3.00 min, | Yield: 273 mg, 1.05 |
| | | | | mmol, 84%. |
| | 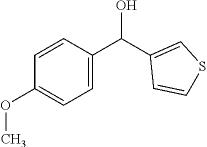 | | | |
| 3D | bis(thiophen-3-yl)methanol | M—OH 178.8 | Method 1: | Intermediate 1D; |
| | | | RT 3.00 min, | Yield: 500 mg, 1.05 |
| | 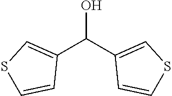 [SMILES: OC(c1ccsc1)c1ccsc1] | | | mmol, 99%. |
| 3E | 4-(Oxan-4-yl)phenyl](thiophen-3- | M—OH 256.9 | Method 1: | Intermediate 1E; |
| | yl)methanol | | RT 3.17 min | Yield: 225.00 mg; 0.74 |
| | | | | mmol; 83%; beige |
| | 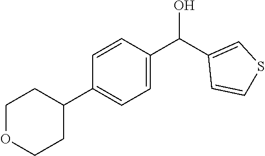 [SMILES: OC(c1ccc(C2CCOCC2)cc1)c1ccsc1] | | | solid |
| 3F | [4-(oxan-4-yl)phenyl](phenyl)methanol | M—OH 251.1. | Method 1: | Intermediate 6B; |
| | | | RT 3.32 min, | Yield: 151.00 mg; 0.55 |
| | 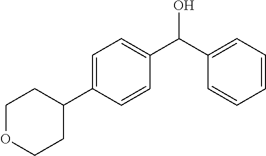 [SMILES: OC(c1ccccc1)c1ccc(C2CCOCC2)cc1] | | | mmol; 73%; beige oil |
| 3G | (4-bromophenyl)(phenyl)methanol | M—OH 244.8 | Method 1: | (4-bromophenyl) |
| | | | (RT 3.6 min, | (phenyl)methanone; |
| | 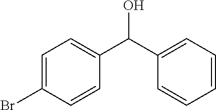 [SMILES: OC(c1ccccc1)c1ccc(Br)cc1] | | | Yield: 6.48 g; 23.89 mmol, yield 89%). |
| 3H | (3-bromophenyl)(phenyl)methanol | M—OH 244.8 | Method 1: | (3-bromophenyl) |
| | | | RT 3.6 min, | (phenyl)methanone |
| | 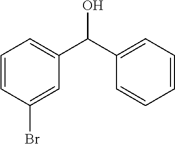 [SMILES: OC(c1ccccc1)c1cccc(Br)c1] | | | Yield: 12.00 g; 43.78 mmol; |

### Intermediate 4A

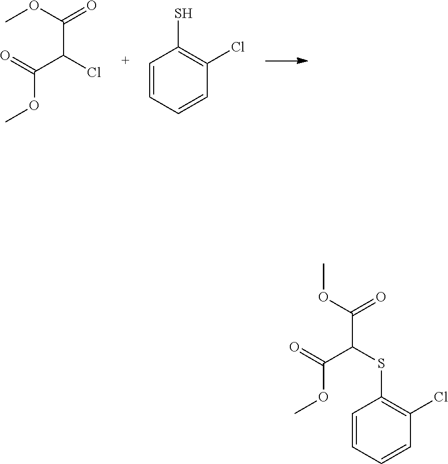

### 1,3-dimethyl 2-[(2-chlorophenyl)sulfanyl]propanedioate

2-Chlorobenzenethiol (5.79 ml; 48.03 mmol; 1.00 eq.) is slowly added to a mixture of 1,3-dimethyl 2-chloropropanedioate 6.13 ml; 48.03 mmol; 1.00 eq.) and anhydrous triethylamine (8.04 ml; 57.64 mmol; 1.20 eq.) in anhydrous DCM (60.00 ml) and stirred at room temperature overnight.

Then the white precipitate is filtered off and the filtrate is washed with 2M NaOH aq. The additional portion of white solid is filtered off. The combined aqueous layers are extracted with DCM. The aqueous layers are combined with white precipitate, acidified using 2M HCl and extracted with DCM. All combined organic layers are dried over MgSO₄ and evaporated to afford 1,3-dimethyl 2-[(2-chlorophenyl)sulfanyl]propanedioate (9.15 g; 32.31 mmol; 67%; beige crystals). Method 1: 3.52 min, p 96.7%, M−1 272.8.

The following compounds are prepared by the procedure for Intermediate 4A, using the appropriate starting materials:

| Inter- | | | | |
|---|---|---|---|---|
| mediate | | | | Starting materials |
| No. | Name and structure | mz | RT | and yield |
| 4B | 1,3-dimethyl 2-[(2-nitrophenyl)sulfanyl]propanedioate | (M − 1) | Method 1: | 2- |
| | | 283.85 | RT 3.21, P | nitrobenzenethiol; |
| | 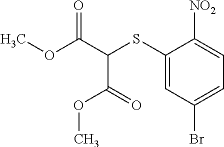 [SMILES: COC(=O)C(Sc1cc(Br)ccc1[N+](=O)[O-])C(=O)OC] | | 96.0% | Yield: 243.0 mg, 0.79 mmol, 44.6% |
| 4C | 1,3-dimethyl 2-[(2-nitro-5- | (M − 1) | Method 1: | 2-nitro-5- |
| | bromo)sulfanyl]propanedioate | 361.8 | RT 3.56 | bromobenzenethiol; |
| | | | min, P | Yield: 1.51 g, |
| | 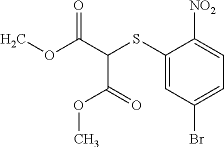 [SMILES: COC(=O)C(Sc1cc(Br)ccc1[N+](=O)[O-])C(=O)OC] | | 98% | 4.06 mmol, 59% |
| 4D | 1,3-dimethyl 2-[(2- | (M − 1) | Method 1: | 2- |
| | bromoophenyl)sulfanyl]propanedioate | 316.80 | RT 3.55 | Bromobenzenethiol; |
| | | | min, P | Yield: 347.5 mg, |
| | 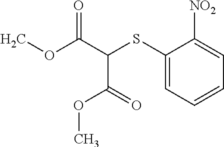 [SMILES: COC(=O)C(Sc1ccccc1[N+](=O)[O-])C(=O)OC] | | 99% | 1.08 mmol, 30% |

### Intermediate 5A

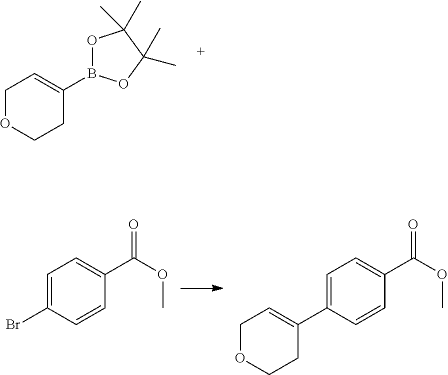 [SMILES: CC1(C)OB(C2=CCOCC2)OC1(C)C ; COC(=O)c1ccc(Br)cc1 ; COC(=O)c1ccc(C2=CCOCC2)cc1]

### Methyl 4-(3,6-dihydro-2H-pyran-4-yl)benzoate

Methyl 4-bromobenzoate (450.00 mg; 2.09 mmol; 1.00 eq.), 4(4,4,5,5-Tetramethyl-[1,3,2]dioxaborolan-2-yl)-3,6-dihydro-2H-pyran (483.57 mg; 2.30 mmol; 1.10 eq.) and potassium carbonate (584.26 mg; 4.19 mmol; 2.00 eq.) are added to a mixture of 10 mL of dioxane and 1.5 mL of water. The resulting mixture is purged with argon for 10 min and then tetrakis(triphenylphosphine)palladium(0) (241.81 mg; 0.21 mmol; 0.10 eq.) is added. Subsequently RM is purged with argon for additional 10 min and heated overnight at 90 C. Then the reaction mixture is diluted with AcOEt and filtered thorough pad of celite. The filtrate is washed with water, brine, dried over Na₂SO₄, filtered and evaporated under reduced pressure. The yellow residue is purified by FCC (AcOEt/Hexane 0=>1:4) to afford 4-(3,6-dihydro-2H-pyran-4-yl)benzoate (481.20 mg; 2.20 mmol; 100%; light brown semi-solid). Method 1: RT 3.24 min, p 100%, M+1+MeCN 259.9 The following compound is prepared by the procedure for Intermediate 5A, using the appropriate starting materials:

| Inter- | | | | |
|---|---|---|---|---|
| mediate | | | | Starting materials |
| No. | Name and structure | mz | RT | and yield |
| 5B | 4-(4-benzoylphenyl)-3,6-dihydro-2H-pyran | M + 1 265.1 | Method | (4-bromophenyl) |
| | | | 1: RT | (phenyl)methanone |
| | 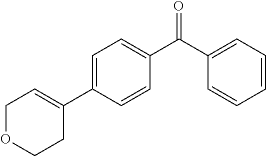 [SMILES: O=C(c1ccccc1)c1ccc(C2=CCOCC2)cc1] | | 3.79 min, p 100% | and 4(4,4,5,5- Tetramethyl- [1,3,2]dioxaborolan- 2-yl)-3,6-dihydro- 2H-pyran; Yield: 263.00 mg; 1.00 mmol; 76% yellow solid |

### Intermediate 6A

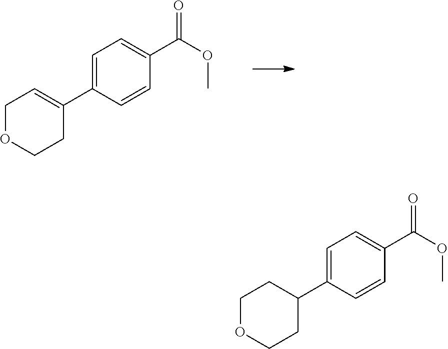 [SMILES: COC(=O)c1ccc(C2=CCOCC2)cc1 ; COC(=O)c1ccc(C2CCOCC2)cc1]

### Methyl 4-(oxan-4-yl)benzoate

4-(3,6-Dihydro-2H-pyran-4-yl)-benzoic acid methyl ester (Intermediate 5A) (420.00 mg; 1.92 mmol; 1.00 eq.) is dissolved in a mixture of methanol (10.00 ml) and DCM (5.00 ml) in the Parr hydrogenation reaction flask and purged with argon. Subsequently the palladium 10% on carbon (40.96 mg; 0.04 mmol; 0.02 eq.) is added to the RM in argon atmosphere. Then the reaction vessel is filled with hydrogen and the reaction mixture is shaken for 3 h at rt, at 2.5 Bar hydrogen pressure. Then catalyst is filtered off, washed with methanol and filtrate is evaporated to give methyl 4-(oxan-4-yl)benzoate (374.00 mg; 1.63 mmol; 85%; white solid). Method 1: RT 315 min, p 96.5%, M+1+MeCN 262.0.

The following compound is prepared by the procedure for Intermediate 6A, using the appropriate starting materials:

| Inter- | | | | |
|---|---|---|---|---|
| mediate | | | | Starting materials |
| No. | Name and structure | mz | RT | and yield |
| 6B | 4-(4-benzoylphenyl)oxane | M + 1 267.3. | Method 3: | Intermediate 5B; |
| | | | RT: 13.8 min, | Yield: 220.00 mg; |
| | 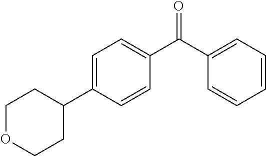 [SMILES: O=C(c1ccccc1)c1ccc(C2CCOCC2)cc1] | | p 90%, | 0.74 mmol; 75%; beige solid |

### Intermediate 7A

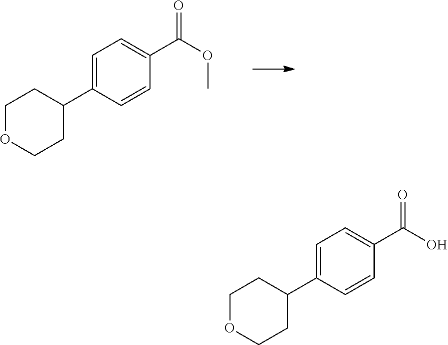 [SMILES: COC(=O)c1ccc(C2CCOCC2)cc1 ; O=C(O)c1ccc(C2CCOCC2)cc1]

### Intermediate 1.3 4-(Oxan-4-yl)benzoic acid

Methyl 4-(oxan-4-yl)benzoate (Intermediate 6A) (370.00 mg; 1.61 mmol; 1.00 eq.) is stirred in the mixture of 5M aq. NaOH (3.23 ml; 16.13 mmol; 10.00 eq.) and MeOH (5.00 ml; 123.28 mmol) at 50 C for 60 min.

Then RM is acidified with 2 M HCl to pH<3, and extracted with AcOEt. The organic extract is washed twice with brine, dried over Na₂SO₄, and evaporated to afford 4-(oxan-4-yl)benzoic acid (326.00 mg; 1.50 mmol; 93%; white solid) Method 1: RT 2.55 min, p: 95%, M−1 204.9.

### Intermediate 9A

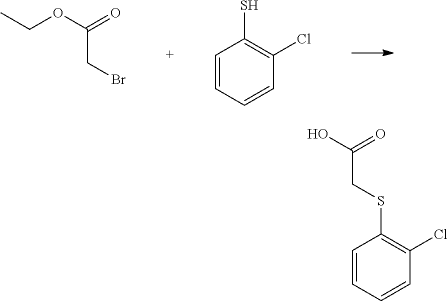

### 2-[(2-chlorophenyl)sulfanyl]acetic acid

To a solution of 2-chlorothiophenole (1.50 ml; 13.11 mmol; 1.00 eq.) and ethyl bromoacetate (1.60 ml; 13.71 mmol; 1.05 eq.) in ethanol (35.00 ml) potassium hydroxide (2.72 g; 41.21 mmol; 3.14 eq.) is added. After approx. 30 min. additional 20 mL of ethanol is added. The reaction mixture is stirred overnight at RT under argon atmosphere. Then 40 mL of water is added and the reaction mixture is stirred for additional 3 h. Then ethanol is evaporated under reduced pressure and the residue is diluted with 50 mL of water and acidified to pH 1 with aq. 2M HCl. Then the RM is cooled and white crystals of (2-[(2-chlorophenyl)sulfanyl]acetic acid are filtered off (2.70 g; 13.31 mmol; 100%; white powder) Method 1: RT 2.92 min, p 99.8%, M−1 200.85

The following compounds are prepared by the procedure for Example 9A, using the appropriate starting materials:

| Inter- | | | | |
|---|---|---|---|---|
| mediate | | | | |
| No. | Name and structure | mz | RT | Starting materials and yield |
| 9B | 2-(2-chlorophenoxy)acetic acid | M − 1 184.9 | Method 1: | 2-chlorophenol and ethyl |
| | | | RT 2.66 | bromoacetate |
| | 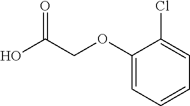 [SMILES: O=C(O)COc1ccccc1Cl] | | min, p 100%. | Yield: 0.584 g, 3.13 mmol, 80%. |

### Intermediate 10

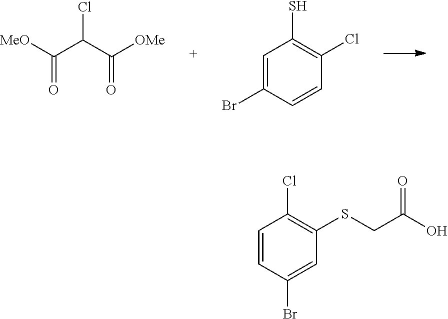 [SMILES: COC(=O)C(Cl)C(=O)OC ; Sc1cc(Br)ccc1Cl ; O=C(O)CSc1cc(Br)ccc1Cl]

### 2-[(5-bromo-2-chlorophenyl)sulfanyl]acetic acid

To a solution of 2-chloromalonic acid dimethyl ester (0.57 ml; 4.47 mmol; 1.00 eq.) and triethylamine (0.75 ml; 5.37 mmol; 1.20 eq.) in anhydrous dichloromethane (5.00 ml) the solution of 5-Bromo-2-chloro-benzenethiol (1.00 g; 4.47 mmol; 1.00 eq.) in dichloromethane (15.00 ml) is slowly added. The resulting mixture is stirred overnight at rt, then washed successively with water (20 ml), NaHCO3 sat. (20 ml) and 5% KHSO4 (20 ml). The organic fraction is dried over magnesium sulphate and concentrated in vacuo. The crude product is purified by flash column chromatography (20% EtOAc in Hexane) to give 2-[(5-Bromo-2-chlorophenyl)sulfanyl]malonic acid dimethyl ester (1.50 g; 3.78 mmol; 84.4%; clear colorless oil). Method 1: 3.87 min, p 88.6%, M−1 352.9.

To the mixture of methanol (20 mL) and 5M aq. NaOH (7.67 ml; 38.37 mmol; 13 eq.) the 2-[(5-bromo-2-chlorophenyl)sulfanyl]malonic acid dimethyl ester (1.50 g; 3.78 mmol; 1.00 eq.) is added. The reaction mixture is stirred at reflux overnight. Then the reaction mixture is cooled down and washed with DCM (50 ml), then acidified by aq. conc. HCl to pH=2 and extracted with EtOAc (3*100 ml). The organic fractions are combined, dried over MgSO4 and concentrated in vacuo to give 5-Bromo-2-chloro-phenylsulfanyl)-acetic acid (1.17 g; 3.32 mmol; 100%; beige oil). Method 1: RT 3.31 min, p 80%, M−1 280.7.

### Intermediate 11

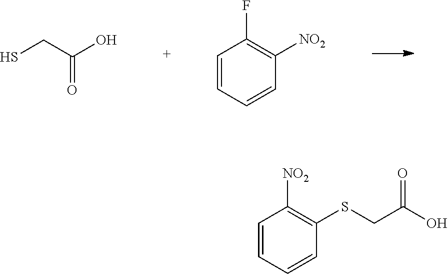 [SMILES: O=C(O)CS ; O=[N+]([O-])c1ccccc1F ; O=C(O)CSc1ccccc1[N+](=O)[O-]]

### 2-Nitrophenylsulfanyl)acetic acid

A mixture of fluoro-2-nitrobenzene (0.70 ml; 6.64 mmol; 1.00 eq.), mercaptoacetic acid (0.51 ml; 7.30 mmol; 1.10 eq.), potassium carbonate (2752.17 mg; 19.91 mmol; 3.00 eq.) and DMF (15.00 ml) is heated at 70° C. for 4.5 hours. The mixture is poured into 200 mL of water and the aqueous phase is washed with 10 mL of ethyl acetate. The aqueous phase is acidified with 1M HCl and extracted three times with 100 ml of DCM. The combined organic layers are dried over MgSO₄ and the solvent is evaporated. Water (10 mL) is added to the residue and the precipitated solid is filtered off, washed with cold water and dried under vacuo to give 1.25 g, 5.85 mmol, 88%(2-Nitrophenylsulfanyl)acetic acid 1.25 g, 5.85 mmol, 88% as yellow powder.

Method 1: RT 2.82 min, p 100%, M−1 212.2.

### Intermediate 12A

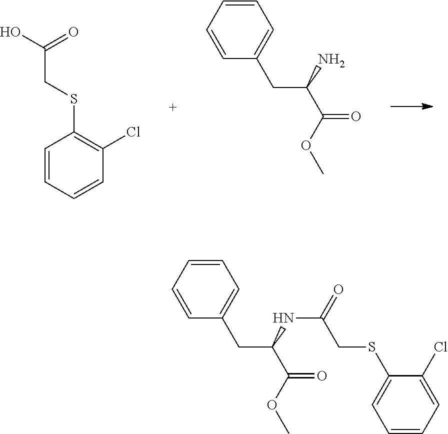 [SMILES: O=C(O)CSc1ccccc1Cl ; COC(=O)[C@H](N)Cc1ccccc1 ; COC(=O)[C@@H](Cc1ccccc1)NC(=O)CSc1ccccc1Cl]

### Methyl (2S)-2-{2-[(2-chlorophenyl)sulfanyl]acetamido}-3-phenylpropanoate

A mixture of 2-[(2-chlorophenyl)sulfanyl]acetic acid (Intermediate 9A) (200.00 mg; 0.98 mmol; 1.00 eq.) and 4-(4,6-Dimethoxy-[1,3,5]triazin-2-yl)-4-methylmorpholin-4-ium chloride (545.09 mg; 1.97 mmol; 2.00 eq.) in anhydrous DMF (4.00 ml) is stirred at RT for 10 min. The resulting suspension is added to a mixture of (S)-phenylalanine methyl ester hydrochloride (212.42 mg; 0.80 mmol; 0.81 eq.), triethylamine (0.42 ml; 2.95 mmol; 3.00 eq.) in anhydrous DMF (3.00 ml) and stirred over weekend at rt. Then ethyl acetate (40 ml) is added and the resulting mixture is washed with 1M HCl (20 mL), water (20 mL) and brine (2×20 mL). The organic layer is dried over anhydrous sodium sulfate, filtered, evaporated under reduced pressure and the resulting residue is purified using FCC (silica, hexane->hexane—ethyl acetate 20%) to afford methyl (2S)-2-{2-[(2-chlorophenyl)sulfanyl]acetamido}-3-phenylpropanoate (268.7 mg; 0.73 mmol; 73.8%). Method 1: RT 3.69 min, p 98.4%, M+1 363.9.

The following compounds are prepared by the procedure for Example 12A, using the appropriate starting materials:

| Inter- | | | | |
|---|---|---|---|---|
| mediate | | | | Starting materials |
| No. | Name and structure | mz | RT | and yield |
| 12B | methyl (2R)-2-{2-[(2- | M + 1 = 363.9 | Method | (R)-phenylalanine |
| | chlorophenyl)sulfanyl]acetamido}-3- | | 1: RT | methyl ester |
| | phenylpropanoate | | 3.66 | hydrochloride and |
| | | | min, p | Intermediate 9A; |
| | 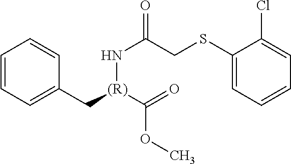 | | 96%,. | Yield: 97 mg, 0.24 mmol, 19%. |
| 12C | methyl (S)-2-{2-[(2- | M + 1 440.05 | Method | (S)-diphenylalanine |
| | chlorophenyl)sulfanyl]acetamido}-3,3- | | 1: RT | methyl ester |
| | diphenylpropanoate | | 4.00 | hydrochloride and |
| | | | min, | Intermediate |
| | 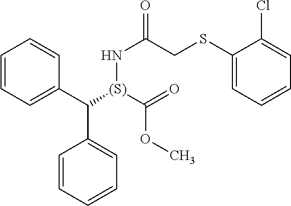 | | p | 9A, Yield: 283 mg, 0.57 mmol, 74%. |
| 12D | methyl 2-[2-(2-chlorophenoxy)acetamido]-3- | M + 1 = 348.0 | Method | Phenylalanine methyl |
| | phenylpropanoate | | 1: RT | ester hydrochloride |
| | | | 3.67 | and Intermediate 9B, |
| | 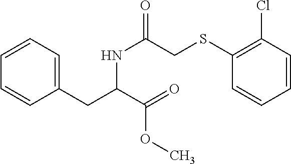 [SMILES: COC(=O)C(Cc1ccccc1)NC(=O)CSc1ccccc1Cl] | | min, p 52.8% | Yield: 145 mg, 0.22 mmol, 41% |
| 12E | methyl 2-{2-[(5-bromo-2- | M + 1 441.95 | Method | Phenylalanine methyl |
| | chlorophenyl)sulfanyl]acetamido}-3- | | 1: RT | ester hydrochloride |
| | phenylpropanoate | | 3.95 | and Intermediate 10, |
| | | | min, | Yield: 238 mg, 0.5 |
| | 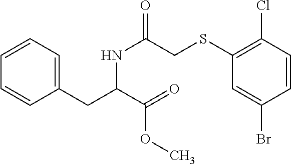 [SMILES: COC(=O)C(Cc1ccccc1)NC(=O)CSc1cc(Br)ccc1Cl] | | P 77.7% | mmol, 73% |
| 12F | methyl 2-{2-[(2- | M + 1 375.05 | Method | Phenylalanine methyl |
| | nitrophenyl)sulfanyl]acetamido}-3- | | 1: RT | ester hydrochloride |
| | phenylpropanoate | | 3.44 | and Intermediatee 11, |
| | | | min, | Yield: 172 mg, 0.39 |
| | 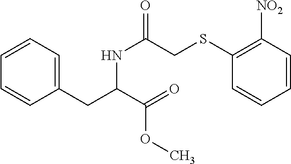 [SMILES: COC(=O)C(Cc1ccccc1)NC(=O)CSc1ccccc1[N+](=O)[O-]] | | p 85.0% | mmol, 42% |
| 12G | methyl 3-(4-bromophenyl)-2-{2-[(2- | M + 1 = 517.95 | Method | 4- |
| | chlorophenyl)sulfanyl]acetamido}-3- | M − 1 = 516.05 | 1: 4.26 | bromodiphenylalanine |
| | phenylpropanoate | | min, p | methyl ester |
| | | | | hydrochloride and |
| | 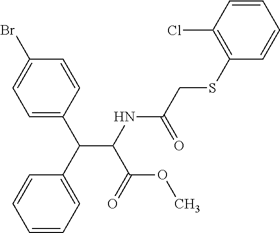 [SMILES: COC(=O)C(NC(=O)CSc1ccccc1Cl)C(c1ccccc1)c1ccc(Br)cc1] | | | intermediate 9A; Yield: 1.22 g, 2.29 mmol, 75% |

### Examples Biological Results:

Examples are tested in selected biological assays one or more times. When tested more than once, data are reported as average values, wherein the average value, also referred to as the mean value, represents the sum of obtained values divided by the number of times tested.

### LDHA and LDHB Assays:

LDHA and LDHB inhibitory activity of compounds of the present invention is quantified employing the decrease in fluorescence of the cofactor—NADH (being the result of oxidation of NADH to NAD+), over the reaction. NADH has fluorescence excitation and emission maxima at 340 nm and 460 nm, respectively, whereas the oxidized form, NAD+, shows no fluorescence. All the experiments are performed in duplicates in a 96-well plate system (black, flat bottom, non-binding).

The procedure for preparation of the experimental plate is as follows (total volume of the reaction mixture: 200 μL/well):

Conditions for LDHA (Abcam, Cat. No# ab93699) Assay

| Reaction buffer | 100 mM phosphate buffer pH | |
|---|---|---|
| | 7.5 + 0.033% BSA | |
| Preincubation time | 30 | mins |
| (reaction buffer + enzyme) | | |
| Time of reaction | 20 | mins |
| Temperature | 25° | C. (RT) |
| LDHA concentration | 0.25 | nM |
| Km pyruvate | 100 | μM |
| Km NADH | 40 | μM |

| | |
|---|---|
| Detection method | Fluorescence_{340/460 nm} |

Conditions for LDHB (Abcam, Cat. No# ab96765) Assay:

| Reaction buffer | 100 mM phosphate buffer pH | |
|---|---|---|
| | 7.5 + 0.033% BSA | |
| Preincubation time | 30 | mins |
| (reaction buffer + enzyme) | | |
| Time of reaction | 20 | mins |
| Temperature | 25° | C. (RT) |
| LDHB concentration | 0.25 | nM |
| Km pyruvate | 130 | μM |
| Km NADH | 30 | μM |

| | |
|---|---|
| Detection method | Fluorescence_{340/460 nm} |

Mixture of appropriate 1× concentrated reaction buffer (175 μL/well) and 40× concentrated LDHA (5 μL/well); pre-incubate for 30 mins at RT is combined with 20× concentrated pyruvate (10 μL/well) and 20× concentrated NADH (10 μL/well). Tested compounds are dissolved in solvent (DMSO, MeOH, EtOH), and then transferred to the V-bottom 96-well plate (mother plate). Single dilution (for % of inhibition determination) or 8-serial dilutions (for IC50 determination) are prepared using the appropriate solvent. The level of NADH fluorescence is then measured using multimode microplate reader (EnSpire, Perkin Elmer). The reaction should be monitored over 20 minutes with the full plate read performed each 5 minutes. Enzymatic reaction should be run at RT.

IC50 is a quantitative measure that indicates how much of a particular compound (inhibitor) is needed to inhibit a given biological process by half. Compounds are classified according to their IC50 values in the assays described above into three groups:

| | |
|---|---|
| Group A | IC50 is in the range of ≥100 μM to <10 μM |
| Group B | IC50 is in the range of ≥10 μM to <100 μM |
| Group C | IC50 is in the range of ≥100 μM to ≤300 μM |

### Lactate Dehydrogenase a Inhibition:

| | Example | LDHA IC50 |
|---|---|---|
| No. | No. | [μM] |
| 1 | 1A | C |
| 2 | 1B | C |
| 3 | 1C | C |
| 4 | 1D | A |
| 5 | 1E | B |
| 6 | 1F | B |
| 7 | 1G | B |
| 8 | 1H | C |
| 9 | 1I | B |
| 10 | 3A | B |
| 11 | 3B | B |
| 12 | 3C | B |
| 13 | 3D | B |
| 14 | 3E | B |
| 15 | 3F | B |
| 16 | 3G | B |
| 17 | 3H | B |
| 18 | 3I | B |
| 19 | 3J | C |
| 20 | 3K | C |
| 21 | 3L | C |
| 22 | 3M | B |
| 23 | 3N | C |
| 24 | 3O | B |
| 25 | 3P | B |
| 26 | 3Q | B |
| 27 | 3R | B |
| 28 | 3S | B |
| 29 | 3T | B |
| 30 | 4A | B |
| 31 | 5A | B |
| 32 | 6A | B |
| 33 | 6B | B |
| 34 | 6C | B |
| 35 | 6D | A |
| 36 | 6E | >300 |
| 37 | 6F | C |
| 38 | 6G | B |
| 39 | 7A | A |
| 40 | 7B | B |
| 41 | 7C | B |
| 42 | 7D | B |
| 43 | 7E | B |
| 44 | 7F | B |
| 45 | 7G | B |
| 46 | 7H | B |
| 47 | 7I | A |
| 48 | 7J | B |
| 49 | 7K | B |
| 50 | 7L | B |
| 51 | 7M | B |
| 52 | 7N | B |
| 53 | 7O | B |
| 54 | 7P | A |
| 55 | 7Q | B |
| 56 | 7R | B |
| 57 | 7S | A |
| 58 | 7T | A |
| 59 | 8A | A |
| 60 | 8B | B |
| 61 | 8C | B |
| 62 | 8D | B |
| 63 | 8E | C |
| 64 | 8F | B |
| 65 | 8G | B |
| 66 | 8H | B |
| 67 | 8I | A |
| 68 | 8J | B |
| 69 | 8K | A |
| 70 | 8L | A |
| 71 | 8M | B |
| 72 | 8N | A |
| 73 | 8O | A |
| 74 | 8P | A |
| 75 | 8Q | A |
| 76 | 8R | B |
| 77 | 8S | B |
| 78 | 8T | B |
| 79 | 8U | B |
| 80 | 8V | B |
| 81 | 8W | B |
| 82 | 8X | B |
| 83 | 8Y | B |
| 84 | 8Z | B |
| 85 | 8AA | B |
| 86 | 8AB | A |
| 87 | 8AC | B |
| 88 | 8AD | B |
| 89 | 8AE | B |
| 90 | 8AF | B |
| 91 | 8AG | A |
| 92 | 9A | B |
| 93 | 9B | B |
| 94 | 9C | C |
| 95 | 9D | B |
| 96 | 9E | B |
| 97 | 9F | B |
| 98 | 9G | B |
| 99 | 9H | B |
| 100 | 9I | B |
| 101 | 9J | B |
| 102 | 9K | C |
| 103 | 9L | B |
| 104 | 9M | B |
| 105 | 9N | B |
| 106 | 9O | B |
| 107 | 9P | B |
| 108 | 9Q | B |
| 109 | 9R | C |
| 110 | 9S | B |
| 111 | 9T | B |
| 112 | 9U | B |
| 113 | 9V | B |
| 114 | 9W | C |
| 115 | 9X | B |
| 116 | 9Y | B |
| 117 | 9Z | B |
| 118 | 9AA | B |
| 119 | 9AB | B |

### Lactate Dehydrogenase B Inhibition:

| | | LDHB IC50 |
|---|---|---|
| No. | Example No. | [μM] |
| 1 | 8L | A |
| 2 | 1D | C |
| 3 | 7S | B |
| 4 | 8A | A |

These assay results establish that compounds according to the present invention are effective in inhibiting activity of LDHA and LDHB

In each case, and by methods known to those practiced, measurement of cellular lactate production is used to measure the activity of compounds on cancer cells.

Cells are seeded on 96-well plate in complete culture medium (200 μL media/well) at a density of 80,000 SNU-398 cells/well. The following day, eight serial dilutions of the test compound is prepared in complete culture medium and added to cells in a fresh media for 2 h. Following incubation 30 μL of culture medium from each well is transferred to a corresponding well on a new 96-well plate. In parallel Lactate Standard (30 μL/well) (Sigma, Cat. # L7022) is prepared in order to create a standard curve. 60 μL of Lactate Reagent (P000024, M Dialysis AB) is added to each well containing lactate standards or test samples and Incubated in dark for 10 min at RT followed by measurement of OD at 530 nm in a microplate reader.

Background subtracted values derived from the lactate standard and sample readings are used to determine ED50 (or % of inhibition) from data obtained in SNU-398 cellular lactate production assay:

ED50 is a quantitative measure that indicates how much of a particular compound is needed to reduce SNU-398 lactate production by half. After normalization to DMSO-control, the value of ED50 is determined by the GraphPad Prism 5.0 [log(inhibitor) vs. normalized response—Variable slope]. For each dose response curve, HillSlope is calculated.

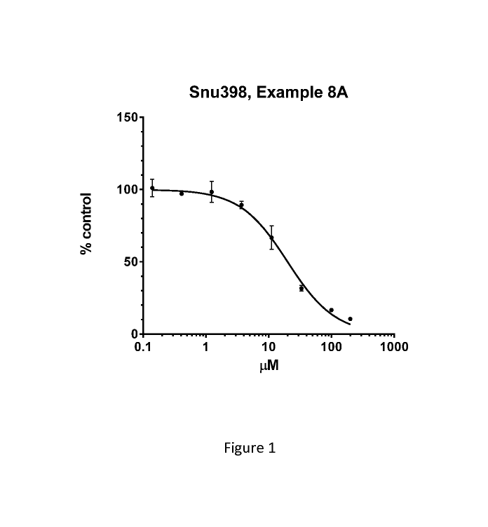

FIG. 1:

Inhibition of lactate production in Snu398 cells treated with Example compound 8A is shown in FIG. 1.

These assay results establish that compounds according to the present invention are effective in inhibiting activity of LDHA and LDHB in cancer cells.

1. Compound of formula (Ia) and/or (Ib) and/or (Ic) 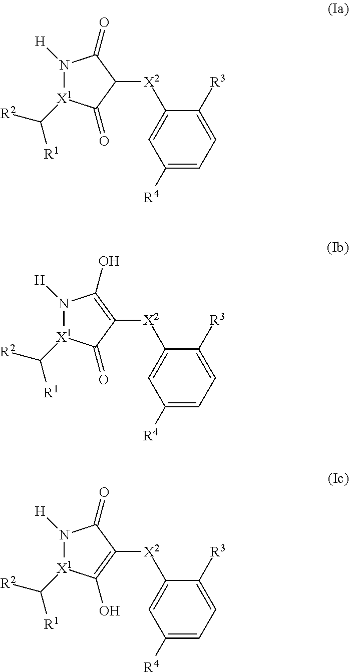
wherein
X¹ denotes N or CH;
X² denotes S or O;
R¹ denotes H, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y};
R² denotes Ar^{X}, Ar^{X}—Ar^{Y}, Ar^{X}-Hetar^{Y}, Ar^{X}-Hetcyc^{Y}, Ar^{X}-L^{Z}-Ar^{Y}, Ar^{X-}LA^{Z}-Ar^{Y}, Ar^{X}-L^{Z}-Hetar^{Y}, Ar^{X}-LA^{Z}-Hetar^{Y}, Ar^{X}-L^{Z}-Hetcyc^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-L^{Z}-Ar^{Y}, Hetar^{X}-LA^{Z}-Ar^{Y}, Hetar^{X}-L^{Z}-Hetar^{Y}, Hetar^{X}-LA^{Z}-Hetar^{Y}, Hetar^{X}-L^{Z}-Hetcyc^{Y}, Hetar^{X}-LA^{Z}-Hetcyc^{Y};
R³ denotes Hal, —CN, —NO₂;
R⁴ denotes H, Hal, LA^{X}, CA^{X}, —CN, NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8};
Ar^{X} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{X1}, R^{X2}, R^{X3};
Ar^{Y} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{Y1}, R^{Y2}, R^{Y3};
Hetar^{X} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or tri-substituted with independently from each other R^{X1}, R^{X2}, R^{X3};
Hetar^{Y} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or tri-substituted with independently from each other R^{Y1}, R^{Y2}, R^{Y3};
Hetcyc^{X} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{X4}, R^{X5}, R^{X6};
Hetcyc^{Y} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{Y4}, R^{Y5}, R^{Y6};
R^{X1}, R^{X2}, R^{X3} denote independently from each other H, Hal, LA^{X}, CA^{X}, —CN, —NO₂, —SO₂NH₂, —SO₂NHR^{X7}, —SO₂NR^{X7}R^{X8}, —NH—SO₂R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—SO₂R^{X9}, —NR^{X7}—SO₂—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}
or
two of R^{X1}, R^{X2}, R^{X3} form a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), —O— wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched C₁₋₆-alkyl or ═O (oxo);
R^{X4}, R^{X5}, R^{X6} denote independently from each other H, Hal, LA^{X}, CA^{X}, —CN, NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O);
R^{Y1}, R^{Y2}, R^{Y3} denote independently from each other H, Hal, LA^{Y}, CA^{Y}, —CN, NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, S(═O)—R^{Y9}, —SO₂—R^{Y9}, —NH₂, —NHR^{Y7}, —NR^{Y7}R^{Y8}, OH, O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8},
or
two of R^{Y1}, R^{Y2}, R^{Y3} form a divalent alkylene chain with 3, 4, 5 chain carbon atoms wherein 1 or 2 non-adjacent CH₂ groups of the divalent alkylene chain may be replaced independently from each other by —N(H)—, —N(C₁₋₆-alkyl)-, —N(—C(═O)—C₁₋₄-alkyl), —O— wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched C₁₋₆-alkyl or ═0 (oxo);
R^{Y4}, R^{Y5}, R^{Y6} denote independently from each other H, Hal, LA^{Y}, CA^{Y}, —CN, NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, S(═O)—R^{Y9}, —SO₂—R^{Y9}, —NH₂, —NHR^{Y7}, —NR^{Y7}R^{Y8}, OH, O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, oxo (═O),
or
two of R^{Y4}, R^{Y5}, R^{Y6} form together with one carbon atom to which they are both attached to a saturated or partially unsaturated ring system A which ring system A is mono- or bicyclic and has 3, 4, 5, 6, 7, 8, 9, 10 ring atoms and may contain no hetero ring atom or 1, 2, 3 hetero ring atoms selected independently from each other N, O and/or S while the remaining ring atoms are carbon atoms wherein that ring system A may be unsubstituted or mono-, di- or trisubstituted with independently from each other R^{A1}, R^{A2}, R^{A3};
L^{Z} denotes —NH—, —NR^{Z7}—, —NH-LA^{Z}-, —NR^{Z7}-LA^{Z}-;
LA^{X} denotes straight-chain or branched C₁₋₆-alkyl or C₂—6-alkenyl that C₁₋₆-alkyl or C₂—6-alkenyl may be unsubstituted or mono-, di- or trisubstituted with independently from each other Hal, —CN, NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —C(═O)—NH—NH₂, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O), wherein 1 or 2 non-adjacent CH₂ groups of the C₁₋₆-alkyl radical or the C₂—6-alkenyl radical may independently from each other be replaced by O, S, N(H) or N—R^{X7} and/or 1 or 2 non-adjacent CH groups of the C₁₋₆-alkyl radical or the C₂₋₆-alkenyl radical may independently from each other be replaced by N;
LA^{Y} denotes straight-chain or branched C₁₋₆-alkyl which may be unsubstituted or mono-, di- or trisubstituted with independently from each other Hal, —CN, NO₂, —SO₂NH₂,
—SO₂NHR^{Y7}, —SO₂NR^{Y7}R^{Y8}, —NH—SO₂—R^{Y9}, —NR^{Y7}—SO₂—R^{Y9}, —S—R^{Y9}, S(═O)—R^{Y9}, —SO₂—R^{Y9}, —NH₂, —NHR^{Y7}, —NR^{Y7}R^{Y8}, OH, O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, oxo (═O), wherein 1 or 2 non-adjacent CH₂ groups of the C₁₋₆-alkyl radical may independently from each other be replaced by O, S, N(H) or N—R^{Y7} and/or 1 or 2 non-adjacent CH groups of the C₁₋₆-alkyl radical may independently from each other be replaced by N;
LA^{Z} denotes a divalent straight-chain or branched C₁₋₆-alkylene radical which alkylene radical may be unsubstituted or mono-, di- or trisubstituted with independently from each other Hal, —CN, NO₂, —SO₂NH₂, —SO₂NHR^{Z7}, —SO₂NR^{Z7}R^{Z8}, —NH—SO₂—R^{Z9}, —NR^{Z7}—SO₂—R^{Z9}, —S—R^{Z9}, S(═O)—R^{Z9}, —SO₂—R^{Z9}, —NH₂, —NHR^{Z7}, —NR^{Z7}R^{Z8}, OH, O—R^{Z9}, —CHO, —C(═O)—R^{Z9}, —COOH, —C(═O)—O—R^{Z9}, —C(═O)—NH₂, —C(═O)—NHR^{Z7}, —C(═O)—NR^{Z7}R^{Z8}, —NH—C(═O)—R^{Z9}, —NR^{Z7}—C(═O)—R^{Z9}, —NH—(C₁₋₃-alkylene)-C(═O)—NH₂, —NH—(C₁₋₃-alkylene)-C(═O)—NHR^{Z7}, —NH—(C₁₋₃-alkylene)-C(═O)—NR^{Z7}R^{Z8}, oxo (═O), wherein 1 or 2 non-adjacent CH₂ groups of that divalent alkylene radical may be replaced independently from each other by O, S or —N(H) and/or 1 or 2 non-adjacent CH groups of that divalent alkylene radical may be replaced by N;
R^{X7}, R^{X8}, R^{X9}, R^{Y7}, R^{Y8}, R^{Y9}, R^{Z7}, R^{Z8}, R^{Z9} denote independently from each other straight-chain or branched C₁₋₆-alkyl, which may be unsubstituted or mono-, di- or trisubstituted with Hal, or a saturated monocyclic carbocycle with 3, 4, 5, 6, 7 carbon atoms,
or
each pair R^{X7} and R^{X8}; R^{Y7} and R^{Y8}; R^{Z7} and R^{Z8} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{A1}, R^{A2}, R^{A3} denote independently from each other H, Hal, Ar^{X}, Hetar^{X}, Hetcyc^{X}, LA^{X}, CA^{X}, —CN, NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O);
CA^{X}, CA^{Y} denote independently from each other a saturated monocyclic carbocycle with 3, 4, 5, 6, 7 carbon atoms which carbocycle may be unsubstituted or mono- or disubstituted with independently from each other R^{CA1}, R^{CA2};
R^{CA1}, R^{CA2} denote independently from each other H, Hal, LA^{X}, —CN, NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8} OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O);
Hal denotes F, Cl, Br, I;
or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios.

2. Compound according to claim 1, or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios,
wherein
X¹ denotes N or CH;
X² denotes S or O;
R¹ denotes H, Ar^{X}, Hetar^{X};
R² denotes Ar^{X}, Ar^{X}-Hetar^{Y}, Ar^{X}-Hetcyc^{Y}, Ar^{X}-L^{Z}-Hetar^{Y}, Ar^{X}-L^{Z}-Hetcyc^{Y}, Hetar^{X};
R³ denotes Hal, —CN, —NO₂;
R⁴ denotes H, Hal;
Ar^{X} denotes a mono- or bicyclic aromatic ring system with 6 or 10 ring carbon atoms which ring system may be unsubstituted or mono- or disubstituted with independently from each other R^{X1}, R^{X2};
Hetar^{X} denotes a mono- or bicyclic aromatic ring system with 5, 6, 7, 8, 9, 10, 11, 12 ring atoms wherein 1 or 2 of said ring atoms is/are a hetero atom(s) selected from N, O and/or S and the remaining are carbon atoms, wherein that aromatic ring system may be unsubstituted or mono- or disubstituted with independently from each other R^{X1}, R^{X2};
Hetar^{Y} denotes a mono- or bicyclic aromatic ring system with 5, 6, 7, 8, 9, 10 ring atoms wherein 1, 2 or 3 of said ring atoms is/are a hetero atom(s) selected from N, O and/or S and the remaining are carbon atoms, wherein that aromatic ring system may be unsubstituted or mono- or disubstituted with independently from each other R^{Y1}, R^{Y2};
Hetcyc^{Y} denotes a saturated or partially unsaturated mono-, bi- or tricyclic heterocycle with 3, 4, 5, 6, 7, 8, 9, 10, 11, 12 ring atoms wherein 1, 2 or 3 ring atom(s) is/are heteroatom(s) selected from N, O and/or S and the remaining ring atoms are carbon atoms, wherein that heterocycle may be unsubstituted or mono- or disubstituted with R^{Y4}, R^{Y5};
R^{X1}, R^{X2} denote independently from each other H, Hal, LA^{X}, —CN, —NO₂, —NH₂, OH, O—R^{X9}, —COOH, —C(═O)—O—R^{X9}, or
R^{X1} and R^{X2} form a divalent alkylene chain with 3, 4, 5 chain carbon atoms wherein 1 or 2 of non-adjacent CH₂ groups of the divalent alkylene chain may be replaced independently from each other by —O—, which divalent alkylene chain may be unsubstituted or mono- or di-substituted with independently from each other straight-chain or branched C₁₋₆-alkyl or ═O (oxo);
R^{Y1}, R^{Y2} denote independently from each other H, Hal, LA^{Y}, OH, O—R^{Y9},
or
R^{Y1} and R^{Y2} form a divalent alkylene chain with 3, 4, 5 chain carbon atoms wherein 1 or 2 non-adjacent CH₂ groups of the divalent alkylene chain may be replaced independently from each other by —O—, which divalent alkylene chain may be unsubstituted or mono- or di-substituted with independently from each other straight-chain or branched C₁₋₆-alkyl or ═O (oxo);
R^{Y4}, R^{Y5} denote independently from each other H, Hal, LA^{Y}, CA^{Y}, —OH, O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9},
or
R^{Y4} and R^{Y5} form together with one carbon atom to which they are both attached to a saturated or partially unsaturated ring system A which ring system A is monocyclic and has 4, 5, 6 ring atoms and may contain no hetero ring atom or 1 or 2 hetero ring atoms selected independently from each other N, O and/or S while the remaining ring atoms are carbon atoms wherein that ring system A may be unsubstituted or monosubstituted with R^{A1};
L^{Z} denotes —NH—, —NR^{Z7}—, —NH-LA^{Z}-, —NR^{Z7}-LA^{Z}-;
LA^{X} denotes straight-chain or branched C₁₋₆-alkyl or C₂₋₆-alkenyl that C₁₋₆-alkyl or C₂₋₆-alkenyl may be unsubstituted or monosubstituted with independently from each other —COOH, —C(═O)—O—R^{X9}, —C(═O)—NH₂, —C(═O)—NHR^{X7}, —C(═O)—NR^{X7}R^{X8}, —C(═O)—NH—NH₂, and/or mono-, di- or trisubstituted with Hal;
LA^{Y} denotes straight-chain or branched C₁₋₆-alkyl which may be unsubstituted or monosubstituted with independently from each other —NH₂, —NHR^{Y7}, —NR^{Y7}R^{Y8}, OH, O—R^{Y9}, —NH—C(═O)—R^{Y9}, —NR^{Y7}—C(═O)—R^{Y9}, and/or mono-, di- or trisubstituted with Hal;
LA^{Z} denotes a divalent straight-chain or branched C₁₋₆-alkylene radical;
R^{X7}, R^{X8}, R^{X9}, R^{Y7}, R^{Y8}, R^{Y9}, R^{Z7} denote independently from each other straight-chain or branched C₁₋₆-alkyl, which may be unsubstituted or mono-, di- or trisubstituted with Hal;
R^{A1} denotes H, Hal, LA^{X} or CA^{X};
CA^{X}, CA^{Y} denote independently from each other a saturated monocyclic carbocycle with 3, 4, 5, 6, 7 carbon atoms which carbocycle may be unsubstituted or mono- or disubstituted with independently from each other R^{CA1}, R^{CA2};
R^{CA1}, R^{CA2} denote independently from each other H, Hal, LA^{X};
Hal denotes F, Cl, Br, I.

3. Compound according to any of the preceding claims, or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios,
wherein
R³ denotes Cl, Br or NO₂;
R⁴ denotes H, Cl or Br.

4. Compound according to any of the preceding claims, or derivatives, N-oxides, prodrugs, solvates, tautomers or stereoisomers, including enantiomers and diastereomers, thereof as well as the physiologically acceptable salts of each of the foregoing, including mixtures thereof in all ratios,
wherein
R¹ and R² are structurally different.

5. Compound according to any of the preceding claims, or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios,
wherein
R¹ denotes H, Ar^{X1} or Hetar^{X1};
Ar^{X1} denotes phenyl which is unsubstituted or mono-substituted with R^{X1a};
Hetar^{X} denotes a monocyclic aromatic ring system with 5 or 6 ring atoms wherein 1 or 2 of said ring atoms is/are a hetero atom(s) selected from N, O and/or S and the remaining are carbon atoms, wherein that aromatic ring system may be unsubstituted or monosubstituted with independently from each other R^{X1b};
R^{X1a} and R^{X1b} denote independently from each other H, Cl or Br.

6. Compound according to claim 5, or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios,
wherein
R¹ denotes H, Ar^{X1} or Hetar^{X1};
Ar^{X1} denotes unsubstituted phenyl; and
Hetar^{X1} denotes unsubstituted thienyl.

7. Compound according to any of the preceding claims, or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios,
wherein
R² denotes Ar^{X2}, Ar^{X2}-Hetar^{Y2}, Ar^{X2}-Hetcyc^{Y2}, Ar^{X2}-L^{Z2}-Hetar^{Y2}, Ar^{X2}-L^{Z2}-Hetcyc^{Y2}, Hetar^{X2};
Ar^{X2} denotes phenyl or naphthyl which phenyl or naphthyl may be unsubstituted or mono- or disubstituted with independently from each other R^{X1c}, R^{X2c};
Hetar^{X2} denotes a mono aromatic ring system with 5 or 6 ring atoms wherein 1 or 2 of said ring atoms is/are a hetero atom(s) selected from N, O and/or S and the remaining are carbon atoms, wherein that aromatic ring system may be unsubstituted or monosubstituted with independently from each other R^{X1d};
Hetar^{Y2} denotes a monocyclic aromatic ring system with 5 or 6 ring atoms wherein 1 or 2 of said ring atoms is/are a hetero atom(s) selected from N, O and/or S and the remaining are carbon atoms, wherein that aromatic ring system may be unsubstituted or mono- or disubstituted with independently from each other R^{Y1a}, R^{Y2a};
Hetcyc^{Y2} denotes a saturated or partially unsaturated mono- or bicyclic heterocycle with 4, 5, 6, 7 or 8 ring atoms wherein 1 or 2 atom(s) is/are heteroatom(s) selected from N and/or O and the remaining ring atoms are carbon atoms, wherein that heterocycle may be unsubstituted or mono- or disubstituted with R^{Y4a}, R^{Y5a};
L^{Z2} denotes —NH— or —NH-LA^{Z2}-;
R^{X1c}, R^{X2c} denote independently from each other H, Hal, LA^{X2c}, —ON, —NO₂, —NH₂, OH, O—R^{X9c}, —COOH, —C(═O)—O—R^{X9c}
or
R^{X1c} and R^{X2c} form a divalent alkylene chain with 3 or 4 chain carbon atoms wherein 2 of non-adjacent CH₂ groups of the divalent alkylene chain may be replaced by —O—;
R^{X1d} denotes H or Hal;
R^{Y1a}, R^{Y2a} denote independently from each other H, LA^{Y2a}, OH, O—R^{Y9a};
R^{Y4a}, R^{Y5a} denote independently from each other H, LA^{Y2b}, CA^{Y2}, —OH, —O—R^{Y9a}, —C(═O)—NH₂,
or
R^{Y4a} and R^{Y5a} form together with one carbon atom to which they are both attached to a saturated ring system A² which ring system A² is monocyclic and has 4 or 5 ring atoms and may contain no hetero ring atom or 1 hetero ring atom being O while the remaining ring atoms are carbon atoms;
LA^{X2c} denotes C₂-alkenyl that is monosubstituted with —C(═O)—O—R^{X9c}, —C(═O)—NH₂, —C(═O)—NH—NH₂;
LA^{Y2a} denotes straight-chain or branched C₁₋₄-alkyl;
LA^{Y2b} denotes straight-chain or branched C₁₋₄-alkyl which may be unsubstituted or monosubstituted with —NR^{Y7a}R^{Y8a}, O—R^{Y9a}, —NH—C(═O)—R^{Y9a}, and/or mono-, di- or trisubstituted with Hal;
LA^{Z2} denotes a divalent straight-chain C₁₋₄-alkylene radical;
CA^{Y2} denotes a saturated monocyclic carbocycle with 3, 4, 5, 6, 7 carbon atoms;
R^{Y7a} and R^{Y7b} denote independently from each other straight-chain or branched C₁₋₄-alkyl;
R^{X9c} denotes methyl or ethyl, which may be unsubstituted or mono-, di- or trisubstituted with Hal;
R^{Y9a} denotes straight-chain or branched C₁₋₄-alkyl;
Hal denotes F, Cl, Br.

8. Compound according to claim 7, or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios,
wherein
R² denotes phenyl, chlorophenyl, 4-chlorophenyl, 3-chlorophenyl, 2-chlorophenyl, bromophenyl, 3-bromophenyl, 4-bromophenyl, methoxyphenyl, 2-methoxyphenyl, 3-methoxyphenyl, 4-methoxyphenyl, aminophenyl, 4-aminophenyl, trifluormethoxyphenyl, 2-trifluormethoxymethyl, nitrophenyl, 2-nitrophenyl, 3-nitrophenyl, 4-nitrophenyl, cyanophenyl, 4-cyanophenyl, 3-cyanophenyl, hydroxyphenyl, 4-hydroxyphenyl, carboxyphenyl (phenyl-COOH), 3-carboxyphenyl, methoxycarbonylphenyl (phenyl-COOCH₃), 3-methoxycarbonylphenyl, methylphenyl, 3-methylphenyl, 3-ethoxy-3-oxo-prop-1-enylphenyl, 4-[3-ethoxy-3-oxo-prop-1-enyl]phenyl, 3-amino-3-oxo-prop-1-enylphenyl, 4-[3-amino-3-oxo-prop-1-enyl]phenyl, 3-hydrazino-3-oxo-prop-1-enylphenyl, 4-[3-hydrazino-3-oxo-prop-1-enyl]phenyl, 1,3-benzodioxol-4-yl, naphthyl, 1-naphthyl; hydroxypyridyl-phenyl, 3-(6-hydroxy-3-pyridyl)phenyl, 4-(6-hydroxy-3-pyridyl)phenyl, pyridylphenyl, 3-(3-pyridyl)phenyl, 3-(4-pyridyl)phenyl, 4-(3-pyridyl)phenyl, 4-(4-pyridyl)phenyl, methoxypyridyl-phenyl, 4-(6-methoxy-3-pyridyl)phenyl, pyrazolylphenyl, 4-(1H-pyrazol-4-yl)phenyl, 3-(1H-pyrazol-4-yl)phenyl, dimethylpyrazolylphenyl, 4-(3,5-dimethyl-1H-pyrazol-4-yl)phenyl, dimethylisoxazolylphenyl, 4-(3,5-dimethylisoxazol-4-yl)phenyl, 4-(1H-pyrazol-3-yl)phenyl; tetrahydropyranylphenyl, 4-tetrahydropyran-4-ylphenyl, piperidylphenyl], 4-(1-piperidyl)phenyl, hydroxypiperidylphenyl, 4-(4-hydroxy-1-piperidyl)phenyl, methoxypiperidylphenyl, 4-(4-methoxy-1-piperidyl)phenyl, methoxypyrrolidinylphenyl, 4-(3-methoxypyrrolidin-1-yl)phenyl, morpholinophenyl, 3-morpholinophenyl, 4-morpholinophenyl, cyclopropylmorpholinylphenyl, 3-(2-cyclopropylmorpholin-4-yl)phenyl, 4-(2-cyclopropylmorpholin-4-yl)phenyl, trifluoromethylmorpholinphenyl, 4-(2-trifluoromethylmorpholin-4-yl)phenyl, (dimethylamino)methyl-morpholinylphenyl, 3-[2-[(dimethylamino)methyl]morpholin-4-yl]phenyl, 4-[2-[(dimethylamino)methyl]morpholin-4-yl]phenyl, acetamidomethylmorpholinylphenyl, 3-[2-(acetamidomethyl)-morpholin-4-yl]phenyl, 4-[2-(acetamidomethyl)morpholin-4-yl]phenyl, methoxymethylmorpholinylphenyl, 3-[2-(methoxy-methyl)morpholin-4-yl]phenyl, 4-[2-(methoxymethyl)morpholin-4-yl]phenyl, carbamoylmorpholinylphenyl, 3-(2-carbamoylmorpholin-4-yl)phenyl, 4-(2-carbamoylmorpholin-4-yl)phenyl, dimethylmorpholinylphenyl, 3-(2,2-dimethylmorpholin-4-yl)phenyl, 4-(2,2-dimethylmorpholin-4-yl)phenyl, 3-(2-oxa-5-azabicyclo[2.2.1]heptan-5-yl)phenyl, 4-(2-oxa-5-azabicyclo[2.2.1]heptan-5-yl)phenyl, 3-(2-oxa-5-azabicyclo[2.2.2]octan-5-yl)phenyl, 4-(2-oxa-5-azabicyclo[2.2.2]octan-5-yl)phenyl, 1,4-oxazepanylphenyl, 3-(1,4-oxazepan-4-yl)phenyl, 4-(1,4-oxazepan-4-yl)phenyl, 4-(6-oxa-9-azaspiro[4.5]decan-9-yl)phenyl, 4-(2-oxa-6-azaspiro[3.3]heptan-6-yl)phenyl, 4-(3,6-dihydro-2H-pyran-4-yl)phenyl; pyrazolylaminophenyl, 4-(1H-pyrazol-4-ylamino)phenyl, (2-hydroxy-4-pyridyl)aminophenyl], 4-[(2-hydroxy-4-pyridyl)amino]phenyl], (6-hydroxy-3-pyridyl)aminophenyl, 4-[(6-hydroxy-3-pyridyl)-amino]phenyl; oxetan-3-ylaminophenyl, 4-(oxetan-3-ylamino)-phenyl, tetrahydrofuran-3-ylaminophenyl, 4-(tetrahydrofuran-3-ylamino)phenyl, tetrahydropyran-4-ylaminophenyl, 4-(tetrahydropyran-4-ylamino)phenyl, tetrahydropyran-3-ylaminophenyl, 4-(tetrahydropyran-3-ylamino)phenyl, tetrahydropyranylmethyl-aminophenyl, 4-(tetrahydropyran-4-ylmethylamino)phenyl; thienyl, 2-thienyl, 3-thienyl, chlorothienyl, 5-chloro-2-thienyl, pyridyl, 2-pyridyl, 3-pyridyl, 4-pyridyl.

9. Compound according to any of claims 1 to 8, or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios,
wherein
X¹ denotes N;
X² denotes S.

10. Compound according to any of the preceding claims, as well as the physiologically acceptable salts thereof, the compound being selected from the group consisting of:
1-[(4-chlorophenyl)(thiophen-3-yl)methyl]-4-[(2-chlorophenyl)sulfanyl]-pyrazolidine-3,5-dione
1-[(3-chlorophenyl)(thiophen-3-yl)methyl]-4-[(2-chlorophenyl)sulfanyl]-pyrazolidine-3,5-dione
1-[(4-bromophenyl)(thiophen-3-yl)methyl]-4-[(2-chlorophenyl)sulfanyl]-pyrazolidine-3,5-dione
4-[(2-chlorophenyl)sulfanyl]-1-{[4-(oxan-4-yl)phenyl](thiophen-3-yl)methyl}pyrazolidine-3,5-dione
4-[(2-chlorophenyl)sulfanyl]-1-{[4-(oxan-4-yl)phenyl](phenyl)methyl}-pyrazolidine-3,5-dione
4-[(2-chlorophenyl)sulfanyl]-1-[(4-methoxyphenyl)(thiophen-3-yl)methyl]-pyrazolidine-3,5-dione
1-[bis(thiophen-3-yl)methyl]-4-[(2-chlorophenyl)sulfanyl]pyrazolidine-3,5-dione
1-[(4-bromophenyl)(phenyl)methyl]-4-[(2-chlorophenyl)sulfanyl]-pyrazolidine-3,5-dione
1-[(3-bromophenyl)(phenyl)methyl]-4-[(2-chlorophenyl)sulfanyl]-pyrazolidine-3,5-dione
4-[(2-chlorophenyl)sulfanyl]-1-{[4-(dimethyl-1,2-oxazol-4-yl)phenyl]-(thiophen-3-yl)methyl}pyrazolidine-3,5-dione
4-[(2-chlorophenyl)sulfanyl]-1-{[4-(3,5-dimethyl-1H-pyrazol-4-yl)phenyl]-(thiophen-3-yl)methyl}pyrazolidine-3,5-dione
4-[(2-chlorophenyl)sulfanyl]-1-{[4-(1H-pyrazol-4-yl)phenyl](thiophen-3-yl)methyl}pyrazolidine-3,5-dione
4-[(2-chlorophenyl)sulfanyl]-1-{[4-(1H-pyrazol-3-yl)phenyl](thiophen-3-yl)methyl}pyrazolidine-3,5-dione
4-[(2-chlorophenyl)sulfanyl]-1-{[3-(1H-pyrazol-4-yl)phenyl](thiophen-3-yl)methyl}pyrazolidine-3,5-dione
4-[(2-chlorophenyl)sulfanyl]-1-{[4-(pyridin-4-yl)phenyl](thiophen-3-yl)methyl}pyrazolidine-3,5-dione
4-[(2-chlorophenyl)sulfanyl]-1-{[4-(pyridin-3-yl)phenyl](thiophen-3-yl)methyl}pyrazolidine-3,5-dione
4-[(2-chlorophenyl)sulfanyl]-1-{[4-(2-methoxypyridin-4-yl)phenyl]-(thiophen-3-yl)methyl}pyrazolidine-3,5-dione
4-[(2-chlorophenyl)sulfanyl]-1-{[4-(3,6-dihydro-2H-pyran-4-yl)phenyl]-(thiophen-3-yl)methyl}pyrazolidine-3,5-dione ethyl (2E)-3-[4-({4-[(2-chlorophenyl)sulfanyl]-3,5-dioxopyrazolidin-1-yl}(thiophen-3-yl)methyl)phenyl]prop-2-enoate
4-[(2-chlorophenyl)sulfanyl]-1-{phenyl[4-(pyridin-3-yl)phenyl]methyl}-pyrazolidine-3,5-dione
4-[(2-chlorophenyl)sulfanyl]-1-{phenyl[4-(pyridin-4-yl)phenyl]methyl}-pyrazolidine-3,5-dione
4-[(2-chlorophenyl)sulfanyl]-1-{[4-(5-hydroxypyridin-2-yl)phenyl](phenyl)-methyl}pyrazolidine-3,5-dione
4-[(2-chlorophenyl)sulfanyl]-1-{phenyl[4-(1H-pyrazol-3-yl)phenyl]-methyl}pyrazolidine-3,5-dione
4-[(2-chlorophenyl)sulfanyl]-1-{phenyl[4-(1H-pyrazol-4-yl)phenyl]-methyl}pyrazolidine-3,5-dione
4-[(2-chlorophenyl)sulfanyl]-1-{phenyl[3-(pyridin-4-yl)phenyl]methyl}-pyrazolidine-3,5-dione
4-[(2-chlorophenyl)sulfanyl]-1-{phenyl[3-(pyridin-3-yl)phenyl]methyl}-pyrazolidine-3,5-dione
4-[(2-chlorophenyl)sulfanyl]-1-{[3-(6-oxo-1,6-dihydropyridin-3-yl)phenyl]-(phenyl)methyl}pyrazolidine-3,5-dione
4-[(2-chlorophenyl)sulfanyl]-1-{phenyl[3-(1H-pyrazol-4-yl)phenyl]-methyl}pyrazolidine-3,5-dione
4-[(2-chlorophenyl)sulfanyl]-1-{[4-(3,6-dihydro-2H-pyran-4-yl)phenyl]-methyl}pyrazolidine-3,5-dione
(2E)-3-[4-({4-[(2-chlorophenyl)sulfanyl]-3,5-dioxopyrazolidin-1-yl}-(thiophen-3-yl)methyl)phenyl]prop-2-enamide
3-[4-({4-[(2-chlorophenyl)sulfanyl]-3,5-dioxopyrazolidin-1-yl}(thiophen-3-yl)methyl)phenyl]propanehydrazide
(5S)-5-benzyl-3-[(2-chlorophenyl)sulfanyl]pyrrolidine-2,4-dione
(5R)-5-benzyl-3-[(2-chlorophenyl)sulfanyl]pyrrolidine-2,4-dione
3-[(2-chlorophenyl)sulfanyl]-5-(diphenyl methyl)pyrrolidine-2,4-dione
5-benzyl-3-(2-chlorophenoxy)pyrrolidine-2,4-dione
5-benzyl-3-[(5-bromo-2-chlorophenyl)sulfanyl]pyrrolidine-2,4-dione
5-benzyl-3-[(2-nitrophenyl)sulfanyl]pyrrolidine-2,4-dione
5-[(4-bromophenyl)(phenyl)methyl]-3-[(2-chlorophenyl)sulfanyl]-pyrrolidine-2,4-dione
4-[(2-chlorophenyl)sulfanyl]-1-({4-[(oxan-4-yl)amino]phenyl}(phenyl)-methyl)pyrazolidine-3,5-dione
1-[(4-aminophenyl)(phenyl)methyl]-4-[(2-chlorophenyl)sulfanyl]-pyrazolidine-3,5-dione
4-[(2-chlorophenyl)sulfanyl]-1-({4-[(oxetan-3-yl)amino]phenyl}(phenyl)-methyl)pyrazolidine-3,5-dione
4-[(2-chlorophenyl)sulfanyl]-1-({4-[(6-oxo-1,6-dihydropyridin-3-yl)amino]-phenyl}(phenyl)methyl)pyrazolidine-3,5-dione
4-[(2-chlorophenyl)sulfanyl]-1-({4-[(2-oxo-1,2-dihydropyridin-4-yl)amino]-phenyl}(phenyl)methyl)pyrazolidine-3,5-dione
4-[(2-chlorophenyl)sulfanyl]-1-[(4-{[(3R)-oxolan-3-yl]amino}phenyl)-(phenyl)methyl]pyrazolidine-3,5-dione
4-[(2-chlorophenyl)sulfanyl]-1-[phenyl({4-[(1H-pyrazol-4-yl)amino]-phenyl})methyl]pyrazolidine-3,5-dione
4-[(2-chlorophenyl)sulfanyl]-1-[(4-{[(3R)-oxan-3-yl]amino}phenyl)-(phenyl)methyl]pyrazolidine-3,5-dione
4-[(2-chlorophenyl)sulfanyl]-1-[(4-{[(3S)-oxan-3-yl]amino}phenyl)-(phenyl)methyl]pyrazolidine-3,5-dione
4-[(2-chlorophenyl)sulfanyl]-1-[(3-{[(oxan-4-yl)methyl]amino}phenyl)-(phenyl)methyl]pyrazolidine-3,5-dione
4-[(2-chlorophenyl)sulfanyl]-1-[(3-{[(3S)-oxolan-3-yl]amino}phenyl)-(phenyl)methyl]pyrazolidine-3,5-dione
4-[(2-chlorophenyl)sulfanyl]-1-({3-[(oxetan-3-yl)amino]phenyl}(phenyl)-methyl)pyrazolidine-3,5-dione
4-[(2-chlorophenyl)sulfanyl]-1-({3-[(6-oxo-1,6-dihydropyridin-3-yl)amino]-phenyl}(phenyl)methyl)pyrazolidine-3,5-dione
4-[(2-chlorophenyl)sulfanyl]-1-[(3-{[(3S)-oxan-3-yl]amino}phenyl)-(phenyl)methyl]pyrazolidine-3,5-dione
4-[(2-chlorophenyl)sulfanyl]-1-({3-[(2-oxo-1,2-dihydropyridin-4-yl)amino]-phenyl}(phenyl)methyl)pyrazolidine-3,5-dione
4-[(2-chlorophenyl)sulfanyl]-1-({4-[(6-oxo-1,6-dihydropyridin-3-yl)amino]-phenyl}(thiophen-3-yl)methyl)pyrazolidine-3,5-dione
4-[(2-chlorophenyl)sulfanyl]-1-({4-[(1H-pyrazol-4-yl)amino]phenyl}-(thiophen-3-yl)methyl)pyrazolidine-3,5-dione
4-[(2-chlorophenyl)sulfanyl]-1-({4-[(oxan-4-yl)amino]phenyl}(thiophen-3-yl)methyl)pyrazolidine-3,5-dione
4-[(2-chlorophenyl)sulfanyl]-1-({4-[(oxetan-3-yl)amino]phenyl}(thiophen-3-yl)methyl)pyrazolidine-3,5-dione
(5R)-3-[(2-chlorophenyl)sulfanyl]-5-[(S)-{4-[(oxan-4-yl)amino]phenyl}-(phenyl)methyl]pyrrolidine-2,4-dione
4-[(2-chlorophenyl)sulfanyl]-1-{[4-(morpholin-4-yl)phenyl](phenyl)-methyl}pyrazolidine-3,5-dione
4-[(2-chlorophenyl)sulfanyl]-1-{[4-(1,4-oxazepan-4-yl)phenyl](phenyl)-methyl}pyrazolidine-3,5-dione
4-[(2-chlorophenyl)sulfanyl]-1-{phenyl[4-(piperidin-1-yl)phenyl]methyl}-pyrazolidine-3,5-dione
4-[(2-chlorophenyl)sulfanyl]-1-{[4-(4-methoxypiperidin-1-yl)phenyl]-(phenyl)methyl}pyrazolidine-3,5-dione
4-[(2-chlorophenyl)sulfanyl]-1-({4-[(3R)-3-methoxypyrrolidin-1-yl]-phenyl}(phenyl)methyl)pyrazolidine-3,5-dione
4-[(2-chlorophenyl)sulfanyl]-1-({4-[(3S)-3-methoxypyrrolidin-1-yl]-phenyl}(phenyl)methyl)pyrazolidine-3,5-dione
4-[(2-chlorophenyl)sulfanyl]-1-{[4-(4-hydroxypiperidin-1-yl)phenyl]-(phenyl)methyl}pyrazolidine-3,5-dione
4-[(2-chlorophenyl)sulfanyl]-1-[(4-{[(3S)-oxolan-3-yl]amino}phenyl)-(phenyl)methyl]pyrazolidine-3,5-dione
4-[(2-chlorophenyl)sulfanyl]-1-{[3-(1,4-oxazepan-4-yl)phenyl](phenyl)-methyl}pyrazolidine-3,5-dione
4-[(2-chlorophenyl)sulfanyl]-1-{[3-(4-methoxypiperidin-1-yl)phenyl]-(phenyl)methyl}pyrazolidine-3,5-dione
4-[(2-chlorophenyl)sulfanyl]-1-{[3-(morpholin-4-yl)phenyl](phenyl)-methyl}pyrazolidine-3,5-dione
4-[(2-chlorophenyl)sulfanyl]-1-{[4-(morpholin-4-yl)phenyl](thiophen-3-yl)methyl}pyrazolidine-3,5-dione
4-[(2-chlorophenyl)sulfanyl]-1-[(4-{2-oxa-6-azaspiro[3.3]heptan-6-yl}-phenyl)(thiophen-3-yl)methyl]pyrazolidine-3,5-dione
4-[(2-chlorophenyl)sulfanyl]-1-[phenyl({4-[2-(trifluoromethyl)morpholin-4-yl]phenyl})methyl]pyrazolidine-3,5-dione
4-[(2-chlorophenyl)sulfanyl]-1-{[3-(2-cyclopropylmorpholin-4-yl)phenyl]-(phenyl)methyl}pyrazolidine-3,5-dione
4-[(2-chlorophenyl)sulfanyl]-1-[(3-{2-oxa-5-azabicyclo[2.2.1]heptan-5-yl}phenyl)(phenyl)methyl]pyrazolidine-3,5-dione
4-[3-({4-[(2-chlorophenyl)sulfanyl]-3,5-dioxopyrazolidin-1-yl}(phenyl)-methyl)phenyl]morpholine-2-carboxamide
4-[(2-chlorophenyl)sulfanyl]-1-{[3-(2,2-dimethylmorpholin-4-yl)phenyl]-(phenyl)methyl}pyrazolidine-3,5-dione
4-[(2-chlorophenyl)sulfanyl]-1-[(4-{3-oxa-8-azabicyclo[3.2.1]octan-8-yl}phenyl)(phenyl)methyl]pyrazolidine-3,5-dione
4-[4-({4-[(2-chlorophenyl)sulfanyl]-3,5-dioxopyrazolidin-1-yl}(phenyl)-methyl)phenyl]morpholine-2-carboxamide
4-[(2-chlorophenyl)sulfanyl]-1-[(4-{6-oxa-9-azaspiro[4.5]decan-9-yl}-phenyl)(phenyl)methyl]pyrazolidine-3,5-dione
4-[(2-chlorophenyl)sulfanyl]-1-[(4-{₂—[(dimethylamino)methyl]morpholin-4-yl}phenyl)(phenyl)methyl]pyrazolidine-3,5-dione
N-({4-[4-({4-[(2-chlorophenyl)sulfanyl]-3,5-dioxopyrazolidin-1-yl}-(phenyl)methyl)phenyl]morpholin-2-yl}methyl)acetamide
4-[(2-chlorophenyl)sulfanyl]-1-{[4-(2,2-dimethylmorpholin-4-yl)phenyl]-(phenyl)methyl}pyrazolidine-3,5-dione
4-[(2-chlorophenyl)sulfanyl]-1-{[4-(2-cyclopropylmorpholin-4-yl)phenyl]-(phenyl)methyl}pyrazolidine-3,5-dione
4-[(2-chlorophenyl)sulfanyl]-1-({4-[2-(methoxymethyl)morpholin-4-yl]-phenyl}(phenyl)methyl)pyrazolidine-3,5-dione
4-[(2-chlorophenyl)sulfanyl]-1-[(4-{2-oxa-5-azabicyclo[2.2.1]heptan-5-yl}phenyl)(phenyl)methyl]pyrazolidine-3,5-dione
4-[(2-chlorophenyl)sulfanyl]-1-[(3-{3-oxa-8-azabicyclo[3.2.1]octan-8-yl}phenyl)(phenyl)methyl]pyrazolidine-3,5-dione
4-[(2-chlorophenyl)sulfanyl]-1-[(3-{₂—[(dimethylamino)methyl]morpholin-4-yl}phenyl)(phenyl)methyl]pyrazolidine-3,5-dione
4-[(2-chlorophenyl)sulfanyl]-1-({3-[2-(methoxymethyl)morpholin-4-yl]-phenyl}(phenyl)methyl)pyrazolidine-3,5-dione
N-({4-[3-({4-[(2-chlorophenyl)sulfanyl]-3,5-dioxopyrazolidin-1-yl}-(phenyl)methyl)phenyl]morpholin-2-yl}methyl)acetamide
4-[(2-chlorophenyl)sulfanyl]-1-{[4-(morpholin-4-yl)phenyl]methyl}-pyrazolidine-3,5-dione
(5R)-3-[(2-chlorophenyl)sulfanyl]-5-[(S)-[4-(morpholin-4-yl)phenyl]-(phenyl)methyl]pyrrolidine-2,4-dione
1-benzyl-4-[(2-chlorophenyl)sulfanyl]pyrazolidine-3,5-dione
3-({4-[(2-chlorophenyl)sulfanyl]-3,5-dioxopyrazolidin-1-yl}methyl)-benzonitrile
4-[(2-chlorophenyl)sulfanyl]-1-[(3-methylphenyl)methyl]pyrazolidine-3,5-dione
4-[(2-chlorophenyl)sulfanyl]-1-{[2-(trifluoromethoxy)phenyl]methyl}-pyrazolidine-3,5-dione
1-[(2H-1,3-benzodioxol-5-yl)methyl]-4-[(2-chlorophenyl)sulfanyl]-pyrazolidine-3,5-dione
4-[(2-chlorophenyl)sulfanyl]-1-[(naphthalen-1-yl)methyl]pyrazolidine-3,5-dione methyl 3-({4-[(2-chlorophenyl)sulfanyl]-3,5-dioxopyrazolidin-1-yl}-methyl)benzoate
4-({4-[(2-chlorophenyl)sulfanyl]-3,5-dioxopyrazolidin-1-yl}methyl)-benzonitrile
4-[(2-chlorophenyl)sulfanyl]-1-[(4-hydroxyphenyl)methyl]pyrazolidine-3,5-dione
3-({4-[(2-chlorophenyl)sulfanyl]-3,5-dioxopyrazolidin-1-yl}methyl)benzoic acid
4-[(2-chlorophenyl)sulfanyl]-1-[(5-chlorothiophen-2-yl)methyl]-pyrazolidine-3,5-dione
4-[(2-chlorophenyl)sulfanyl]-1-[(thiophen-3-yl)methyl]pyrazolidine-3,5-dione
4-[(2-chlorophenyl)sulfanyl]-1-[(thiophen-2-yl)methyl]pyrazolidine-3,5-dione
4-[(2-chlorophenyl)sulfanyl]-1-[(4-methoxyphenyl)methyl]pyrazolidine-3,5-dione
4-[(2-chlorophenyl)sulfanyl]-1-[(3-methoxyphenyl)methyl]pyrazolidine-3,5-dione
4-[(2-chlorophenyl)sulfanyl]-1-[(2-methoxyphenyl)methyl]pyrazolidine-3,5-dione
4-[(2-chlorophenyl)sulfanyl]-1-[(pyridin-4-yl)methyl]pyrazolidine-3,5-dione
4-[(2-chlorophenyl)sulfanyl]-1-[(pyridin-3-yl)methyl]pyrazolidine-3,5-dione
4-[(2-chlorophenyl)sulfanyl]-1-[(pyridin-2-yl)methyl]pyrazolidine-3,5-dione
1-[(3-chlorophenyl)methyl]-4-[(2-chlorophenyl)sulfanyl]pyrazolidine-3,5-dione
1-[(2-chlorophenyl)methyl]-4-[(2-chlorophenyl)sulfanyl]pyrazolidine-3,5-dione
1-[(4-chlorophenyl)methyl]-4-[(2-chlorophenyl)sulfanyl]pyrazolidine-3,5-dione
4-[(2-chlorophenyl)sulfanyl]-1-[(3-nitrophenyl)methyl]pyrazolidine-3,5-dione
4-[(2-chlorophenyl)sulfanyl]-1-[(2-nitrophenyl)methyl]pyrazolidine-3,5-dione
4-[(2-chlorophenyl)sulfanyl]-1-[(4-nitrophenyl)methyl]pyrazolidine-3,5-dione
1-benzyl-4-[(2-nitrophenyl)sulfanyl]pyrazolidine-3,5-dione
1-benzyl-4-[(5-bromo-2-nitrophenyl)sulfanyl]pyrazolidine-3,5-dione
2-benzyl-4-[(2-bromophenyl)sulfanyl]-5-hydroxy-2,3-dihydro-1H-pyrazol-3-one

11. Process for manufacturing a compound according to any one of claims 1 to 10, or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios, the process being characterized in that
(a) a compound of formula (II) 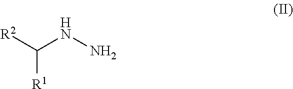
wherein
R¹ and R² are as defined in claim 1 for formulas (Ia), (Ib) and (Ic);
is reacted with a compound of formula (III) 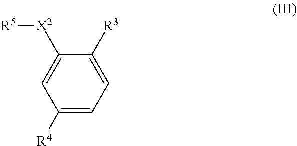
wherein
R³, R⁴ and X² are as defined in claim 1 for formulas (Ia), (Ib) and (Ic);
R⁵ denotes a malonic acid dialkyl ester residue, —CH—(C(O)—O—C₁₋₄alkyl)₂, or a malonic acid dihalide, —CH—(C(O)-Hal)₂ with Hal being Cl or Br;
to yield a compound of formulas (Ia) and/or (Ib) and/or (Ic) 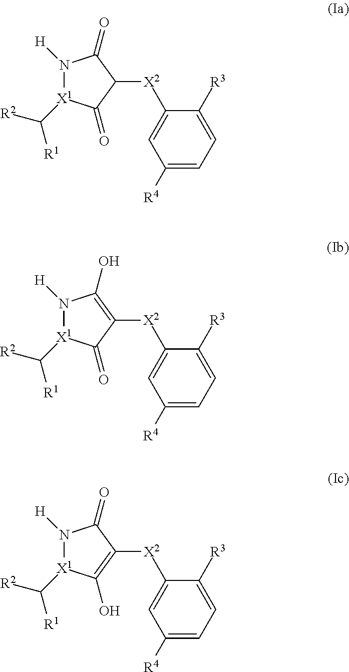
wherein
R¹, R², R³, R⁴ and X² are as defined in claim 1; and
X¹ denotes N;
or
(b) a compound of formula (IV) 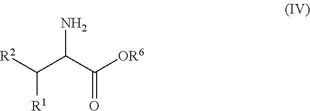
wherein
R¹ and R² are as defined in claim 1 for formulas (Ia), (Ib) and (Ic);
R⁶ denotes C₁₋₄-alkyl;
is reacted with a compound of formula (V) 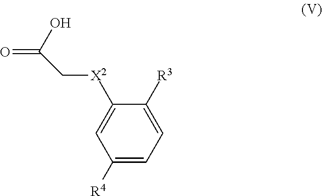
wherein
R³, R⁴ and X² are as defined in claim 1 for formulas (Ia), (Ib) and (Ic);
to first form an amide of formula (VI) 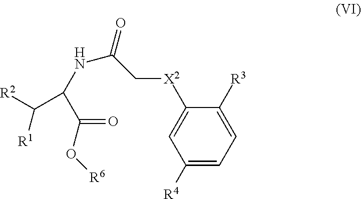
wherein
R¹, R², R³, R⁴, R⁶ and X² are as defined above in this claim;
and then convert that amide of formula (VI) into a compound of formulas (Ia) and/or (Ib) and/or (Ic) 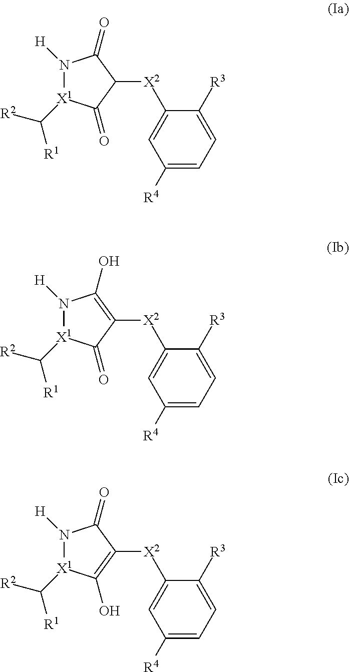
wherein
R¹, R², R³, R⁴ and X² are as defined in claim 1; and
X¹ denotes CH.

12. A pharmaceutical composition comprising at least one compound of formula (Ia) and/or (Ib) and/or (Ic) as defined in any one of claims 1 to 10, or derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios, as active ingredient, together with a pharmaceutically acceptable carrier.

13. The pharmaceutical composition according to claim 12 that further comprises a second active ingredient or its derivatives, N-oxides, prodrugs, solvates, tautomers or stereoisomers thereof as well as the physiologically acceptable salts of each of the foregoing, including mixtures thereof in all ratios, wherein that second active ingredient is other than a compound of formula (Ia) and/or (Ib) and/or (Ic) as defined in any one of claims 1 to 10.

14. Medicament comprising at least one compound of formula (Ia) and/or (Ib) and/or (Ic) as defined in any one of claims 1 to 10, or its derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios.

15. A compound of formula (Ia) and/or (Ib) and/or (Ic) as defined in any one of claims 1 to 10, or its derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios, for use in the prevention and/or treatment of medical conditions that are affected by inhibiting lactate dehydrogenase (LDH), in particular LDHA and/or LDHB.

16. A compound of formula (Ia) and/or (Ib) and/or (Ic) as defined in any one of claims 1 to 10, or its derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios, for use in the prevention and/or treatment of autoimmune diseases, autoinflammatory diseases, metabolic disorders, infective diseases and cancer, in particular central nervous system cancer, cervical cancer, glioblastoma, glioma, myeloid neoplasia, chondrosarcoma, angioimmunoblastic T-cell lymphoma (AITL), cholangiocarcinoma, prostate cancer, leukemia, lymphoma, lymphoid cancer, kidney cancer, hypoxic carcinomas, breast cancer, ovarian cancer, mesothelioma, pancreatic cancer, colon cancer, colorectal cancer, lung cancer, lung adenocarcinomas, non-small cell lung cancer (NSCLC), liver cancer, hepatocellular carcinoma.

17. Set (kit) comprising separate packs of
a) an effective amount of a compound of formula (Ia) and/or (Ib) and/or (Ic) as defined in any one of claims 1 to 10, or its derivatives, N-oxides, prodrugs, solvates, tautomers or stereoisomers, including enantiomers, diastereomers and E/Z-isomers, thereof as well as the physiologically acceptable salts of each of the foregoing, including mixtures thereof in all ratios; and
b) an effective amount of a further active ingredient that further active ingredient not being a compound of formula (Ia) and/or (Ib) and/or (Ic) as defined in any one of claims 1 to 10.
